(19) United States
(12) Patent Application Publication
(10) Pub. No.: US 2006/0287316 A1
(43) Pub. Date: Dec. 21, 2006

(54) METHOD FOR IMPROVING PHARMACOKINETICS OF PROTEASE INHIBITORS AND PROTEASE INHIBITOR PRECURSORS

(75) Inventors: Jinzi J. Wu, Dollard des Ormeaux (CA); Brent Richard Stranix, Pointe-Claire (CA); Michael Ge, St-Laurent (CA); Guy Milot, Blainville (CA); Marco Petrella, Montreal (CA); Chandra Panchal, London (CA)

Correspondence Address: GODFREY & KAHN S.C., 780 NORTH WATER STREET, MILWAUKEE, WI, 53202, US

(73) Assignee: AMBRILIA BIOPHARMA INC., Verdun (CA)

(21) Appl. No.: 11/380,291

(22) Filed: Apr. 26, 2006

Related U.S. Application Data
(60) Provisional application No. 60/675,082, filed on Apr. 27, 2005.

(51) Int. Cl. A61K 31/5375; A61K 31/4965; A61K 31/498; A61K 31/551; A61K 31/445; A61K 31/44; A61K 31/522; A61K 31/47; A61K 31/18

(52) U.S. Cl. 514/237.5; 514/255.06; 514/313; 514/317; 514/357; 514/310; 514/249; 514/602; 514/263.34; 514/220

(57) ABSTRACT

The present invention provides methods for improving the pharmacokinetics of protease inhibitors and protease inhibitor precursors and pharmaceutical composition comprising protease inhibitors or protease inhibitor precursors of formula I and a cytochrome P450 monooxigenase inhibitor; 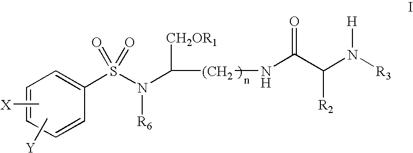
when the compound of formula I comprises an amino group, pharmaceutically acceptable ammonium salts thereof, wherein R₁ may be, for example, (HO)₂P(O)—, (NaO)₂P(O)—, alkyl-CO— or cycloalkyl-CO—, wherein X may be, for example, F, Cl, and Br, and wherein R₂ and R₃ are as defined herein.

### RELATED APPLICATION

The present application seeks priority from U.S. Provisional application 60/675,082, which is incorporated by reference in its entirety, as if fully set forth herein.

### TECHNICAL FIELD OF THE INVENTION

This invention relates to method for improving the pharmacokinetics of protease inhibitors and protease inhibitor precursors and related pharmaceutical compositions. More particularly, the present invention relates to method for improving the pharmacokinetics of protease inhibitors and protease inhibitor precursors by co-administering a cytochrome P450 monooxygenase inhibitor.

### BACKGROUND OF THE INVENTION

Inhibitors of the HIV viral protease have been developed relatively recently and their use began only in 1996. Currently, they are considered the most effective drugs against HIV infection. Unfortunately, most current proteases inhibitors are relatively large hydrophobic molecules that possess rather low bioavailability. A high pill burden is therefore required to attain the therapeutic dose in a patient. This is a deterrent, which too often results in patient non-compliance and inadequate treatment results. This situation leads to sub-optimal therapeutic drug concentration that in turns leads to the development of HIV resistant strains. Consequently, there is an urgent need to improve the solubility and bioavailability of proteases inhibitors.

Examples of improved compounds have been developed in the form of prodrugs of aspartyl protease inhibitors such as described, for example, in U.S. Pat. No. 6,436,989 to Hale et al, the entire content of which is incorporated herein by reference. This patent shows a novel class of molecules characterized by favourable aqueous solubility, high oral bioavailability and facile in vivo generation of the active ingredient. However, it is well known that HIV has the ability to develop resistance to the currently available drugs. Thus, there is a need for alternative HIV protease inhibitors active towards wild-type and resistant viral strains. Thus, molecules derived from current HIV protease inhibitors showing enhanced solubility and bioavailability is desirable to fight resistant viral strains.

A unique class of aromatic derivatives which are inhibitors of aspartyl proteases is described in U.S. Pat. No. 6,632,816 to Stranix et al, the entire content of which is incorporated herein by reference. This patent includes, more particularly, N-synthetic amino acid substituted L-lysine derivatives possessing potent aspartyl protease inhibitory properties. However, it would be advantageous to improve these derivatives by enhancing aqueous solubility and bioavailability in order to reduce the pill burden and to favour patient's compliance. Since it is challenging to generate active protease inhibitors, specifically toward wild-type and resistant strains, the formation of derivatives of original HIV protease inhibitors such as inhibitors described in U.S. Pat. No. 6,632,816 to Stranix et al, known to be active toward resistant strains represents a viable route with considerable advantages. More particularly, generation of compounds and formulations with enhanced aqueous solubility, oral bioavailability, time of duration and formulation properties along with other advantages is desirable in the development of an effective drug. Protease inhibitors with improved pharmacokinetics are therefore desirable.

### SUMMARY OF THE INVENTION

Lysine-based compounds with increased solubility and improved oral bioavailability are described herein and have been described in U.S. patent application Ser. No. 10/902,935 filed on Aug. 2, 2004 and published on Feb. 2, 2006 under No. 2006/0025592A1, the entire content of which is incorporated herein by reference. These compounds may readily be cleaved in vivo to release an active ingredient which has an affinity for aspartyl proteases and which may act as a protease inhibitor. More particularly, the active ingredient may bind, for example, to an HIV aspartyl protease (U.S. Pat. No. 6,632,816) and may inhibit this enzyme. The Lysine-based compounds are also referred herein as a protease inhibitor precursor.

Upon in vivo physiological conditions (e.g., metabolic, enteric and/or gastrointestinal conditions, etc.) the Lysine-based compounds, allow for the release of a protease inhibitor (e.g., aspartyl protease inhibitor). The Lysine-based compounds may thus serve as means for improving the solubility and/or bioavailability of protease inhibitors and therefore may reduce the pill burden and/or reduce dosages needed for inhibition. Improved treatment of HIV-infected patients and favourable patient's compliance may consequently occur.

The Lysine-based compounds described herein may be used alone or in combination with other therapeutic or prophylactic agents for the treatment or prophylaxis of HIV infection.

The active ingredient when released from the Lysine-based compound may act, for example, on aspartyl protease of HIV-1 including mutated and non-mutated HIV-1 viral strain (e.g., NL4.3) as well as on protease of HIV-2 (mutated or non-mutated) or even on protease of related viruses (e.g. retroviruses (e.g. SIV, etc.) etc.).

Other means to increase the pharmacokinetics of Lysine-based compounds described herein or of the active ingredients disclosed in U.S. Pat. No. 6,632,816 to Stranix et al, are investigated herein.

In accordance with the present invention, there is provided a method of improving the pharmacokinetics of a Lysine-based compound or of the active ingredients disclosed in U.S. Pat. No. 6,632,816 to Stranix et al. The method may comprise administering, to an individual in need thereof, a cytochrome P450 monooxygenase inhibitor (or a pharmaceutically acceptable salt thereof) in combination with a Lysine-based compound (or a pharmaceutically acceptable salt thereof) and/or with an active ingredient as disclosed in U.S. Pat. No. 6,632,816 (or a pharmaceutically acceptable salt thereof).

The method may comprise administering the cytochrome P450 monooxygenase inhibitor and the protease inhibitor or protease inhibitor precursor either separately, simultaneously or sequentially. The method may also comprise administering the cytochrome P450 monooxygenase inhibitor and the protease inhibitor or protease inhibitor precursor at different time interval.

For example, when administering a combination of a Lysine-based compound (or a pharmaceutically acceptable salt thereof) and a cytochrome P450 monooxygenase inhibitor (or a pharmaceutically acceptable salt thereof) the two therapeutic agents may be formulated as separate composition which may be administered separately at the same time or at different times, using the same route of administration or different routes of administration, at the same administration site or at different administration sites etc. Alternatively, the therapeutic agents may be administered as a single composition either orally, by injection, etc.

In accordance with the present invention, the CYP450 inhibitor may be a CYP450-3A inhibitor.

Cytochrome P450 monooxygenase inhibitor (CYP450 inhibitor) which may be used to increase the pharmacokinetics of Lysine-based compounds or of the active ingredients disclosed in U.S. Pat. No. 6,632,816 to Stranix et al., include for example, ritonavir (RTV), ketoconazole, fluconazole, nefazodone, fluvoxamine, fluoxetine, macrolide antibiotics, sertraline sulfaphenazole, erythromycin etc.

The present invention further provides a method for treating, preventing, reducing the risk or probability of HIV infection or reducing HIV burden. The invention also provides a method for treating, preventing or reducing the risk or probability of developing AIDS, for delaying the apparition of AIDS or reducing AIDS symptoms. The method may comprise administering a Lysine-based compound (or a pharmaceutically acceptable salt thereof) as described herein or the active ingredients disclosed in U.S. Pat. No. 6,632,816 to Stranix et al, and a CYP450 inhibitor (or a pharmaceutically acceptable salt thereof).

In yet a further aspect, the present invention provides a method of treating, preventing or reducing the risk or probability of HIV infection or of reducing HIV burden. The invention also provides a method for treating, preventing or reducing the risk or probability of developing acquired immunodeficiency syndrome (AIDS), for delaying the apparition of AIDS, or reducing AIDS symptoms. The method may comprise administering at least one compound of formula I, II, IIa, IIb, IIc, IIA, IIA′ or pharmaceutically acceptable salts or derivatives thereof or the active ingredients disclosed in U.S. Pat. No. 6,632,816 to Stranix et al, pharmaceutically acceptable salts or derivatives thereof, or mixture of any of these compounds to a mammal in need thereof in combination with a CYP450 inhibitor.

The present invention also relates in a further aspect thereof to a pharmaceutical composition comprising a) a Lysine-based compound (or a pharmaceutically acceptable salt or derivatives thereof) as described herein or an active ingredient as disclosed in U.S. Pat. No. 6,632,816 to Stranix et al. (or pharmaceutically acceptable salts or derivatives thereof or mixture of any one of these compounds thereof; b) a CYP450 inhibitor (or a pharmaceutically acceptable salt thereof; and c) a pharmaceutically acceptable carrier.

In yet another aspect, the present invention relates to a pharmaceutical composition comprising at least one compound of formula I, II, IIa, IIb, IIc, IIA, IIA′ as described herein (or pharmaceutically acceptable salts or derivatives thereof or an active ingredient as disclosed in U.S. Pat. No. 6,632,816 to Stranix et al., (or pharmaceutically acceptable salts or derivatives thereof or mixture of any of these compounds and a CYP450 inhibitor (or a pharmaceutically acceptable salt thereof. The pharmaceutical composition may also comprise a pharmaceutically acceptable carrier. The pharmaceutical composition may comprise, for example, a pharmaceutically effective amount of such one or more compounds or as applicable, pharmaceutically acceptable ammonium salts thereof.

More particularly, the pharmaceutical composition may consist essentially of at least one compound of formula I, II, IIa, IIb, IIc, IIA, IIA′ as described herein or pharmaceutically acceptable salts or derivatives thereof or the active ingredients disclosed in U.S. Pat. No. 6,632,816 to Stranix et al, or mixture of any of these compounds and a CYP450 inhibitor (or a pharmaceutically acceptable salt thereof).

Additionally, the pharmaceutical composition may consist of at least one compound of formula I, II, IIa, IIb, IIc, IIA, IIA′ as described herein or pharmaceutically acceptable salts or derivatives thereof or the active ingredients disclosed in U.S. Pat. No. 6,632,816 to Stranix et al, or mixture of any of these compounds and a CYP450 inhibitor (or a pharmaceutically acceptable salt thereof.

The present invention also relates in an additional aspect thereof to the use of a combination of a) a Lysine-based compound (or a pharmaceutically acceptable salt thereof) as described herein or the active ingredients disclosed in U.S. Pat. No. 6,632,816 to Stranix et al, (or a pharmaceutically acceptable salt thereof) or mixture of any of these compounds and b) a CYP450 inhibitor (or a pharmaceutically acceptable salt thereof) in the making of a pharmaceutical composition for the treatment or prevention of a retroviral infection (e.g., HTLV, HIV, i.e., HIV-1, HIV-2) or for the treatment or prevention of acquired immunodeficiency syndrome (AIDS).

In an additional aspect, the present invention relates to the use of at least one compound of formula I, II, IIa, IIb, IIc, IIA, IIA′ as described herein or pharmaceutically acceptable salts or derivatives thereof or the active ingredients disclosed in U.S. Pat. No. 6,632,816 to Stranix et al, or mixture of any of these compounds and a CYP450 inhibitor (or a pharmaceutically acceptable salt thereof) in the manufacture of a drug (or pharmaceutical composition) for the treatment or prevention of an HIV infection (for reducing the risk or probability of HIV infection), or again for reducing HIV burden in a mammal in need thereof or for the treatment or prevention of acquired immunodeficiency syndrome (AIDS) (for reducing the risk or probability of developing AIDS) for delaying the apparition of AIDS (symptoms) or for reducing AIDS symptoms in a mammal in need thereof.

A “mammal in need” is to be understood herein, without limitation, as an individual infected with HIV (i.e., at any stage of HIV infection, e.g., primary infection, symptomatic, asymptomatic, AIDS) or at risk of having an HIV infection. A “mammal in need” therefore, may comprise, in addition to individuals who has an acute HIV infection, a chronic HIV infection or AIDS, individuals (e.g., health care worker, an individual who had unprotected sexual intercourse, policemen, etc.) who may have, for example, come into contact with a possible source of contamination with HIV.

The present invention, additionally provides the use of a combination of a Lysine-based compound (or a pharmaceutically acceptable salt thereof) as described herein or the use of an active ingredient disclosed in U.S. Pat. No. 6,632,816 to Stranix et al, (or a pharmaceutically acceptable salt thereof) and a CYP450 inhibitor (or a pharmaceutically acceptable salt thereof) for the treatment, prevention or for reducing the risk or probability of a retroviral infection in a mammal in need thereof.

More particularly, the present invention relates to the use of a combination of at least one compound of formula I, II, IIa, IIb, IIc, IIA, IIA′ as described herein or pharmaceutically acceptable salts or derivatives thereof or the active ingredients disclosed in U.S. Pat. No. 6,632,816 to Stranix et al, (or a pharmaceutically acceptable salt thereof) or mixture of any of these compounds and a CYP450 inhibitor in the treatment, prevention or for reducing the risk or probability of an HIV infection in a mammal in need thereof or for the treatment, prevention or for reducing the risk or probability of developing acquired immunodeficiency syndrome (AIDS).

Exemplary embodiments of Lysine-based compounds encompassed by the present invention and which may be used in combination with a CYP450 inhibitor for the methods, pharmaceutical compositions, kits and uses described herein, may include, for example, a compound of formula I:

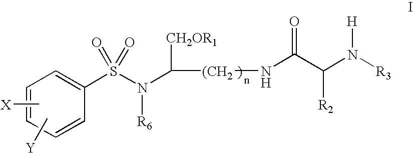

pharmaceutically acceptable salts and derivatives thereof (e.g., for example, when the compound of the present invention comprises an amino group, the pharmaceutically acceptable salt may be an ammonium salt),

wherein n may be, for example, 3 or 4,

wherein X and Y, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —OCF₃, —CN, —NO₂, —NR₄R₅, —NHCOR₄, —OR₄, —SR₄, —COOR₄, —COR₄, and —CH₂OH or X and Y together define an alkylenedioxy group selected from the group consisting of a methylenedioxy group of formula —OCH₂O— and an ethylenedioxy group of formula —OCH₂CH₂O—,

wherein R₆ may be selected, for example, from the group consisting of a straight alkyl group of 1 to 6 carbon atoms, a branched alkyl group of 3 to 6 carbon atoms, a cycloalkylalkyl group having 3 to 6 carbon atoms in the cycloalkyl part thereof and 1 to 3 carbon atoms in the alkyl part thereof,

wherein R₃ may be selected, for example, from the group consisting of H, a straight alkyl group of 1 to 6 carbon atoms, a branched alkyl group of 3 to 6 carbon atoms, a cycloalkyl group of 3 to 6 carbon atoms, and a group of formula R_{3A}—CO—, wherein R_{3A} may be selected, for example, from the group consisting of a straight or branched alkyl group of 1 to 6 carbon atoms (e.g. methyl, ethyl-, propyl, iso-propyl, butyl, iso-butyl, tert-butyl, tert-butyl-CH₂—, etc.), a cycloalkyl group having 3 to 6 carbon atoms (e.g. cyclopropyl-, cyclohexyl-etc.), a cycloalkylalkyl group having 3 to 6 carbon atoms in the cycloalkyl part thereof and 1 to 3 carbon atoms in the alkyl part thereof, (e.g. cyclopropyl-CH₂—, cyclohexyl-CH₂—, etc.), an alkyloxy group of 1 to 6 carbon atoms (e.g. CH₃O—, CH₃CH₂O—, iso-butylO-, tert-butylO-(Boc), etc.), tetrahydro-3-furanyloxy, —CH₂OH, —CF₃, —CH₂CF₃, —CH₂CH₂CF₃, pyrrolidinyl, piperidinyl, 4-morpholinyl, CH₃O₂C—, CH₃O₂CCH₂—, Acetyl-OCH₂CH₂—, HO₂CCH₂—, 3-hydroxyphenyl, 4-hydroxyphenyl, 4-CH₃OC₆H₄CH₂—, CH₃NH—, (CH₃)₂N—, (CH₃CH₂)₂N—, (CH₃CH₂CH₂)₂N—, HOCH₂CH₂NH—, CH₃OCH₂O—, CH₃OCH₂CH₂O—, C₆H₅CH₂O—, 2-pyrrolyl, 2-pyridyl, 3-pyridyl, 4-pyridyl-, 2-pyrazinyl, 2-quinolyl, 3-quinolyl, 4-quinolyl, 1-isoquinolyl, 3-isoquinolyl, 2-quinoxalinyl, a phenyl group of formula

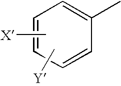

a picolyl group selected from the group consisting of

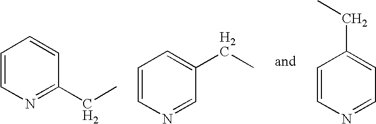

a picolyloxy group selected from the group consisting of

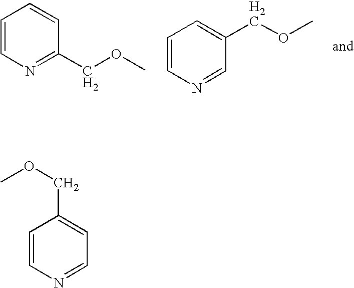

a substituted pyridyl group selected from the group consisting of

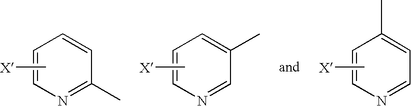

and a group of formula,

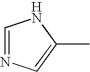

wherein X′ and Y′, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —NO₂, —NR₄R₅, —NHCOR₄, —OR₄, —SR₄, —COOR₄, —COR₄ and —CH₂OH,

wherein R₄ and R₅, the same or different, may be selected, for example, from the group consisting of H, a straight alkyl group of 1 to 6 carbon atoms, a branched alkyl group of 3 to 6 carbon atoms, and a cycloalkyl group of 3 to 6 carbon atoms,

wherein R₂ may be selected, for example, from the group consisting of a diphenylmethyl group of formula IV

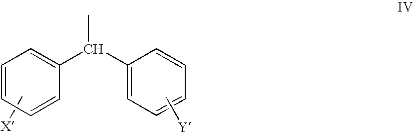

a naphthyl-1-CH₂— group of formula V

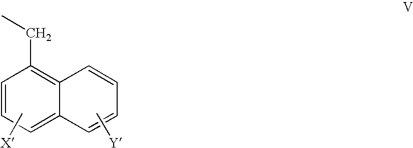

a naphthyl-2-CH₂— group of formula VI

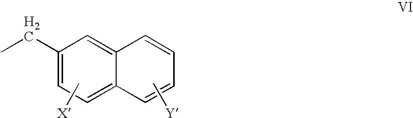

a biphenylmethyl group of formula VI

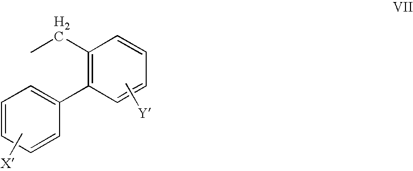

and an anthryl-9-CH₂— group of formula VIII

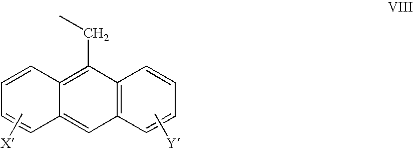

and wherein R₁ may be a cleavable unit (e.g., a physiologically cleavable unit), whereby upon cleavage of the unit, the compound releases a protease inhibitor (an HIV protease inhibitor), provided that R₁ is not H. For example, R₁ may be an enzymatically or metabolically cleavable unit or hydrolysable bond which may be cleaved under enteric and/or gastrointestinal conditions (pH) or other physiological conditions.

In accordance with the present invention, R₁ may be selected, for example, from the group consisting of (HO)₂P(O) and (MO)₂P(O), wherein M is an alkali metal (e.g. Na, K, Cs, etc) or alkaline earth metal (Ca, Mg, etc.).

Further in accordance with the present invention, R₁ may be a group of formula R_{1A}—CO—, wherein R_{1A} may be selected, for example, from the group consisting of a straight or branched alkyl group of 1 to 6 carbon atoms (e.g. methyl, ethyl, propyl, iso-propyl, butyl, iso-butyl, tert-butyl, tert-butyl-CH₂—, etc.), a cycloalkyl group having 3 to 6 carbon atoms (e.g. cyclopropyl-, cyclohexyl- etc.), a cycloalkylalkyl group having 3 to 6 carbon atoms in the cycloalkyl part thereof and 1 to 3 carbon atoms in the alkyl part thereof, (e.g. cyclopropyl-CH₂—, cyclohexyl-CH₂—, etc.), an alkyloxy group of 1 to 6 carbon atoms (e.g. CH₃O—, CH₃CH₂O—, iso-butylO-, tert-butylO—(Boc), etc.), —CH₂OH, CH₃O₂C—, CH₃O₂CCH₂—, Acetyl-OCH₂CH₂—, HO₂CCH₂—, 2-hydroxyphenyl, 3-hydroxyphenyl, 4-hydroxyphenyl, (CH₃)₂NCH₂—, (CH₃)₂CHCH(NH₂)—, HOCH₂CH₂NH—, CH₃OCH₂O—, CH₃OCH₂CH₂O—, 2-pyrrolyl, 2-pyridyl, 3-pyridyl, 4-pyridyl, 1-methyl-1,4-dihydro-3-pyridyl, 2-pyrazinyl, 2-quinolyl, 3-quinolyl, 4-quinolyl, 1-isoquinolyl, 3-isoquinolyl, 2-quinoxalinyl, a phenyl group of formula

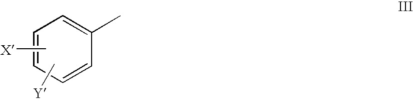

a picolyl group selected from the group consisting of

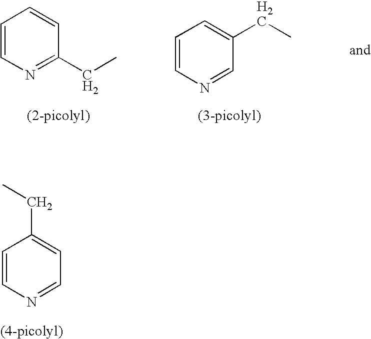

a picolyloxy group selected from the group consisting of

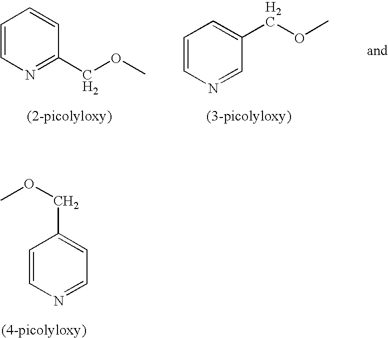

a substituted pyridyl group selected from the group consisting of

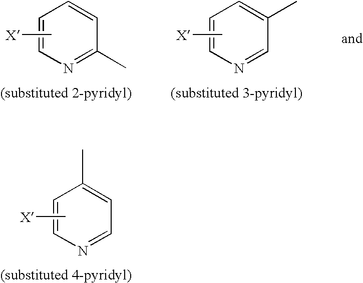

and a group of formula,

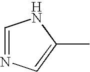

wherein X′, Y′, R₄ and R₅ are as defined herein.

Alternatively, active ingredients disclosed in U.S. Pat. No. 6,632,816 to Stranix et al, which may be used in combination with a CYP450 inhibitor in the methods, pharmaceutical compositions, kits and uses described herein, are also defined by formula I, II, IIa, IIb, IIc, IIA or IIA′, (or a salt thereof), wherein R₁ is H and R₂, R₃, R₄, R₅, R₆, n, X, Y, X′, Y′ are as defined herein.

More particularly, the present invention relates to a pharmaceutical composition which may comprise:

a) a compound of formula I as described herein or a pharmaceutically acceptable salt thereof,

b) a cytochrome P450 monooxigenase inhibitor, and;

c) a pharmaceutically acceptable carrier;

where the compound of formula I is represented by;

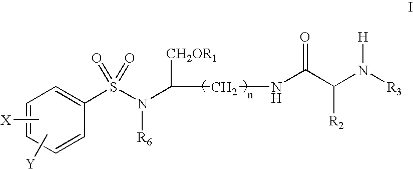

wherein n may be 3 or 4,

wherein X and Y, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —OCF₃, —CN, —NO₂, —NR₄R₅, —NHCOR₄, —OR₄, —SR₄, —COOR₄, —COR₄, and —CH₂OH or X and Y together may define an alkylenedioxy group which may be selected, for example, from the group consisting of a methylenedioxy group of formula —OCH₂O— and an ethylenedioxy group of formula —OCH₂CH₂O—,

wherein R₆ may be selected from the group consisting of a straight alkyl group of 1 to 6 carbon atoms, a branched alkyl group of 3 to 6 carbon atoms, a cycloalkylalkyl group having 3 to 6 carbon atoms in the cycloalkyl part thereof and 1 to 3 carbon atoms in the alkyl part thereof,

wherein R₃ may be selected, for example, from the group consisting of H, a straight alkyl group of 1 to 6 carbon atoms, a branched alkyl group of 3 to 6 carbon atoms, a cycloalkyl group of 3 to 6 carbon atoms, and a group of formula R_{3A}—CO—, where R_{3A} may be selected from the group consisting of a straight or branched alkyl group of 1 to 6 carbon atoms, a cycloalkyl group having 3 to 6 carbon atoms, a cycloalkylalkyl group having 3 to 6 carbon atoms in the cycloalkyl part thereof and 1 to 3 carbon atoms in the alkyl part thereof, an alkyloxy group of 1 to 6 carbon atoms, tetrahydro-3-furanyloxy, —CH₂OH, —CF₃, —CH₂CF₃, —CH₂CH₂CF₃, pyrrolidinyl, piperidinyl, 4-morpholinyl, CH₃O₂C—, CH₃O₂CCH₂—, Acetyl-OCH₂CH₂—, HO₂CCH₂—, 3-hydroxyphenyl, 4-hydroxyphenyl, 4-CH₃OC₆H₄CH₂—, CH₃NH—, (CH₃)₂N—, (CH₃CH₂)₂N—, (CH₃CH₂CH₂)₂N—, HOCH₂CH₂NH—, CH₃OCH₂O—, CH₃OCH₂CH₂O—, C₆H₅CH₂O—, 2-pyrrolyl, 2-pyridyl, 3-pyridyl, 4-pyridyl-, 2-pyrazinyl, 2-quinolyl, 3-quinolyl, 4-quinolyl, 1-isoquinolyl, 3-isoquinolyl, 2-quinoxalinyl, a phenyl group of formula

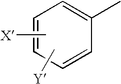

a picolyl group which may be selected from the group consisting of

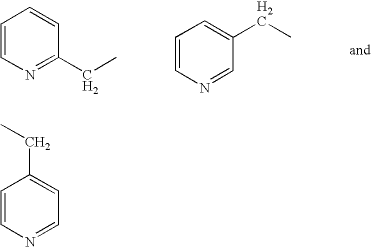

a picolyloxy group which may be selected from the group consisting of

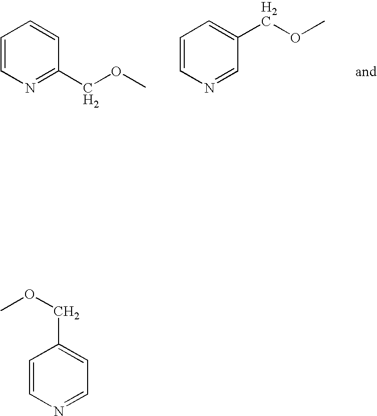

a substituted pyridyl group which may be selected from the group consisting of

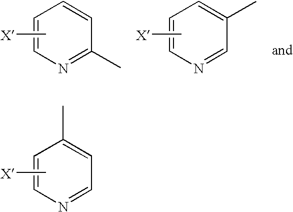

a group of formula

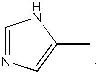

wherein X′ and Y′, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —NO₂, —NR₄R₅, —NHCOR₄, —OR₄, —SR₄, —COOR₄, —COR₄ and —CH₂OH,

wherein R₄ and R₅, the same or different, may be selected from the group consisting of H, a straight alkyl group of 1 to 6 carbon atoms, a branched alkyl group of 3 to 6 carbon atoms, and a cycloalkyl group of 3 to 6 carbon atoms,

wherein R₂ may be selected from the group consisting of a diphenylmethyl group of formula IV

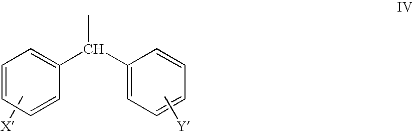

a naphthyl-1-CH₂— group of formula V

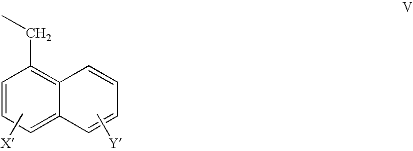

a naphthyl-2-CH₂— group of formula VI

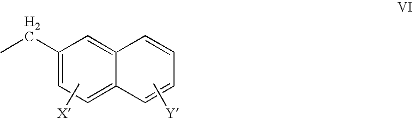

a biphenylmethyl group of formula VII

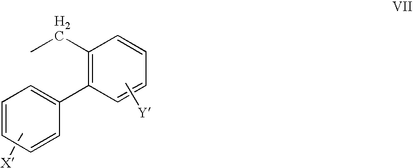

and an anthryl-9-CH₂— group of formula VIII

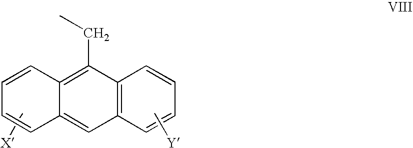

wherein R₁ may be H or a physiologically cleavable unit, whereby upon (in vivo) physiological conditions the compound may be converted into an active protease inhibitor. For example, upon cleavage of the physiologically cleavable unit, the compound may be able to release a protease inhibitor.

In accordance with the present invention, R₁ may be selected, for example, from the group consisting of H, (HO)₂P(O) and (MO)₂P(O) (wherein M may be, for example, an alkali metal or alkaline earth metal) and a group of formula R_{1A}-CO—, where R_{1A} may be selected, for example, from the group consisting of a straight or branched alkyl group of 1 to 6 carbon atoms, a cycloalkyl group having 3 to 6 carbon atoms, a cycloalkylalkyl group having 3 to 6 carbon atoms in the cycloalkyl part thereof and 1 to 3 carbon atoms in the alkyl part thereof, an alkyloxy group of 1 to 6 carbon atom, —CH₂OH, CH₃O₂C—, CH₃O₂CCH₂—, Acetyl-OCH₂CH₂—, HO₂CCH₂—, 2-hydroxyphenyl, 3-hydroxyphenyl, 4-hydroxyphenyl, (CH₃)₂NCH₂—, (CH₃)₂CHCH(NH₂)—, HOCH₂CH₂NH—, CH₃OCH₂O—, CH₃OCH₂CH₂O—, 2-pyrrolyl, 2-pyridyl, 3-pyridyl, 4-pyridyl, 1-methyl-1,4-dihydro-3-pyridyl, 2-pyrazinyl, 2-quinolyl, 3-quinolyl, 4-quinolyl, 1-isoquinolyl, 3-isoquinolyl, 2-quinoxalinyl, a phenyl group of formula

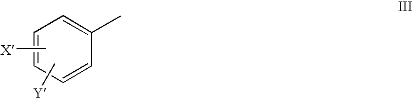

a picolyl group selected from the group consisting of

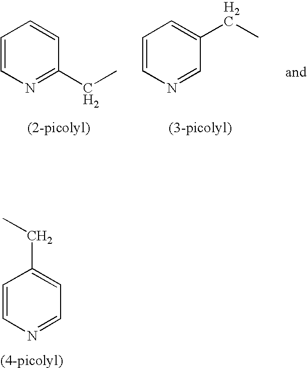

a picolyloxy group selected from the group consisting of

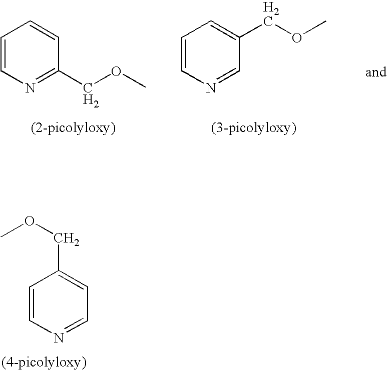

a substituted pyridyl group selected from the group consisting of

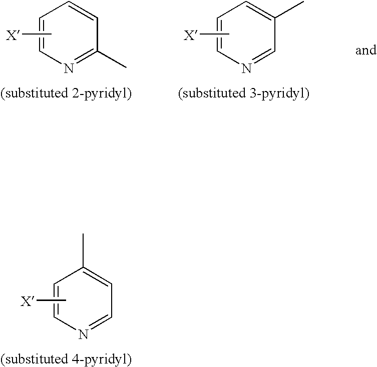

and a group of formula,

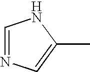

wherein X′, Y′, R₄ and R₅ are as defined herein.

Exemplary embodiments of Lysine-based compounds encompassed by the present invention and which may be used in combination with a CYP450 inhibitor in the methods, pharmaceutical compositions, kits and uses described herein are also defined by a compound of formula II,

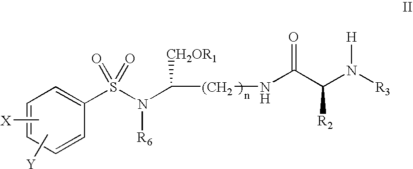

pharmaceutically acceptable salts and derivatives thereof (e.g., for example, when the compound of the present invention comprises an amino group, the pharmaceutically acceptable salt may be an ammonium salt),

wherein n may be 3 or 4,

wherein X and Y, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —OCF₃, —CN, —NO₂, —NR₄R₅, —NHCOR₄, —OR₄, —SR₄, —COOR₄, —COR₄, and —CH₂OH or X and Y together define an alkylenedioxy group selected from the group consisting of a methylenedioxy group of formula —OCH₂O— and an ethylenedioxy group of formula —OCH₂CH₂O—,

wherein R₆ may be selected, for example, from the group consisting of a straight alkyl group of 1 to 6 carbon atoms, a branched alkyl group of 3 to 6 carbon atoms, a cycloalkylalkyl group having 3 to 6 carbon atoms in the cycloalkyl part thereof and 1 to 3 carbon atoms in the alkyl part thereof,

wherein R₃ may be selected, for example, from the group consisting of H, a straight alkyl group of 1 to 6 carbon atoms, a branched alkyl group of 3 to 6 carbon atoms, a cycloalkyl group of 3 to 6 carbon atoms, and a group of formula R_{3A}-CO—, wherein R_{3A} may be selected, for example, from the group consisting of a straight or branched alkyl group of 1 to 6 carbon atoms (e.g. methyl, ethyl-, propyl, iso-propyl, butyl, iso-butyl, tert-butyl, tert-butyl-CH₂—, etc.), a cycloalkyl group having 3 to 6 carbon atoms (e.g. cyclopropyl-, cyclohexyl-etc.), a cycloalkylalkyl group having 3 to 6 carbon atoms in the cycloalkyl part thereof and 1 to 3 carbon atoms in the alkyl part thereof, (e.g. cyclopropyl-CH₂—, cyclohexyl-CH₂—, etc.), an alkyloxy group of 1 to 6 carbon atoms (e.g. CH₃O—, CH₃CH₂O—, iso-butylO-, tert-butylO-(Boc), etc.), tetrahydro-3-furanyloxy, —CH₂OH, —CF₃, —CH₂CF₃, —CH₂CH₂CF₃, pyrrolidinyl, piperidinyl, 4-morpholinyl, CH₃O₂C—, CH₃O₂CCH₂—, Acetyl-OCH₂CH₂—, HO₂CCH₂—, 3-hydroxyphenyl, 4-hydroxyphenyl, 4-CH₃OC₆H₄CH₂—, CH₃NH—, (CH₃)₂N—, (CH₃CH₂)₂N—, (CH₃CH₂CH₂)₂N—, HOCH₂CH₂NH—, CH₃OCH₂O—, CH₃OCH₂CH₂O—, C₆H₅CH₂O—, 2-pyrrolyl, 2-pyridyl, 3-pyridyl, 4-pyridyl, 2-pyrazinyl, 2-quinolyl, 3-quinolyl, 4-quinolyl, 1-isoquinolyl, 3-isoquinolyl, 2-quinoxalinyl, a phenyl group of formula

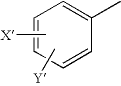

a picolyl group selected from the group consisting of

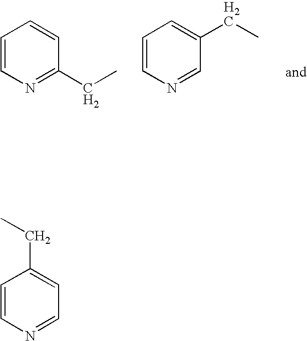

a picolyloxy group selected from the group consisting of

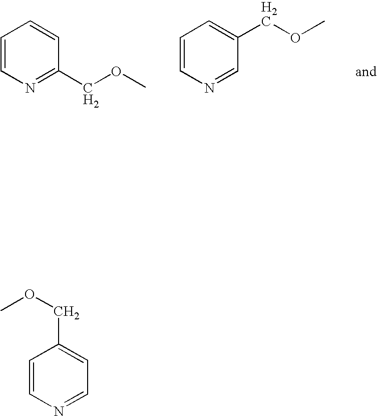

a substituted pyridyl group selected from the group consisting of

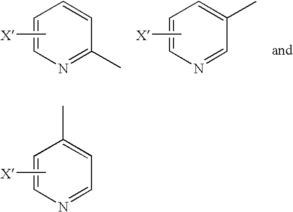

and a group of formula,

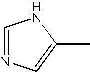

wherein X′ and Y′, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —NO₂, —NR₄R₅, —NHCOR₄, —OR₄, —SR₄, —COOR₄, —COR₄ and —CH₂OH,

wherein R₄ and R₅, the same or different, may be selected, for example, from the group consisting of H, a straight alkyl group of 1 to 6 carbon atoms, a branched alkyl group of 3 to 6 carbon atoms, and a cycloalkyl group of 3 to 6 carbon atoms,

wherein R₂ may be selected from the group consisting of a diphenylmethyl group of formula IV

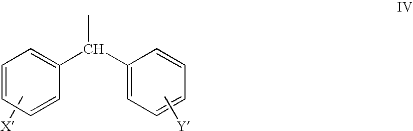

a naphthyl-1-CH₂— group of formula V

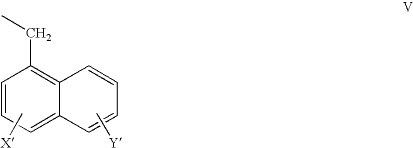

a naphthyl-2-CH₂— group of formula VI

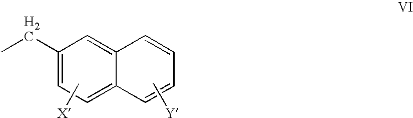

a biphenylmethyl group of formula VII

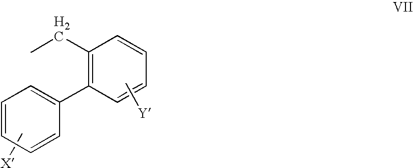

and an anthryl-9-CH₂— group of formula VIII

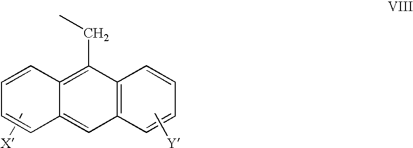

and wherein R₁ may be a physiologically cleavable unit, whereby upon cleavage of the unit the compound may be able to release a protease inhibitor, provided that R₁ is not H.

In accordance with the present invention, R₁ may be selected, for example, from the group consisting of (HO)₂P(O) and (MO)₂P(O), wherein M is an alkali metal (e.g. Na, K, Cs, etc) or alkaline earth metal (Ca, Mg, etc.).

Further in accordance with the present invention R₁ may be a group of formula R_{1A}—CO—, wherein R_{1A} may be selected from the group consisting of a straight or branched alkyl group of 1 to 6 carbon atoms (e.g. methyl, ethyl, propyl, iso-propyl, butyl, iso-butyl, tert-butyl, tert-butyl-CH₂—, etc.), a cycloalkyl group having 3 to 6 carbon atoms (e.g. cyclopropyl-, cyclohexyl- etc.), a cycloalkylalkyl group having 3 to 6 carbon atoms in the cycloalkyl part thereof and 1 to 3 carbon atoms in the alkyl part thereof, (e.g. cyclopropyl-CH₂—, cyclohexyl-CH₂—, etc.), an alkyloxy group of 1 to 6 carbon atoms (e.g. CH₃O—, CH₃CH₂O—, iso-butylO-, tert-butylO-(Boc), etc.), —CH₂OH, CH₃O₂C—, CH₃O₂CCH₂—, Acetyl-OCH₂CH₂—, HO₂CCH₂—, 2-hydroxyphenyl, 3-hydroxyphenyl, 4-hydroxyphenyl, (CH₃)₂NCH₂—, (CH₃)₂CHCH(NH₂)—, HOCH₂CH₂NH—, CH₃OCH₂O—, CH₃OCH₂CH₂O—, 2-pyrrolyl, 2-pyridyl, 3-pyridyl, 4-pyridyl, 1-methyl-1,4-dihydro-3-pyridyl, 2-pyrazinyl, 2-quinolyl, 3-quinolyl, 4-quinolyl, 1-isoquinolyl, 3-isoquinolyl, 2-quinoxalinyl, a phenyl group of formula

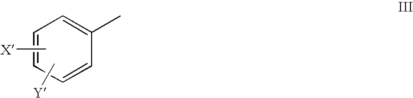

a picolyl group selected from the group consisting of

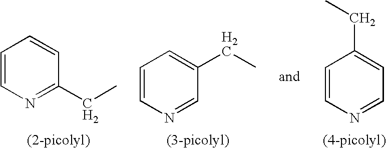

a picolyloxy group selected from the group consisting of

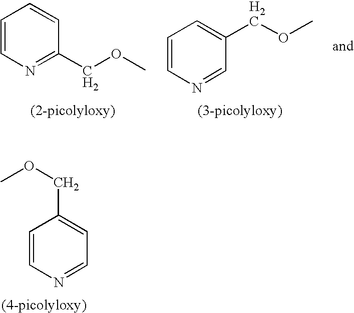

a substituted pyridyl group selected from the group consisting of

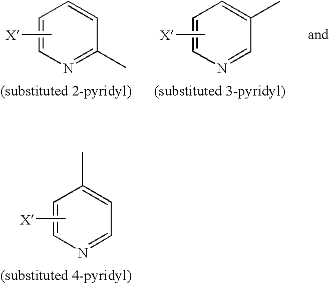

and a group of formula,

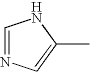

wherein X′, Y′, R₄ and R₅ are as defined herein.

As mentioned herein, active ingredients disclosed in U.S. Pat. No. 6,632,816 to Stranix et al., which may be used in combination with a CYP450 inhibitor in the methods, pharmaceutical compositions, kits and uses described herein, are defined by a compound of formula II (or a salt thereof), wherein R₁ is H and R₂, R₃, R₄, R₅, R₆, n, X, Y, X′, Y′ are as defined herein.

More particularly, the present invention provides in one aspect thereof, a pharmaceutical composition which may comprise;

a) a compound of formula II and pharmaceutically acceptable salts thereof,

b) a cytochrome P450 monooxigenase inhibitor, and;

c) a pharmaceutically acceptable carrier;

where the compound of formula II is represented by;

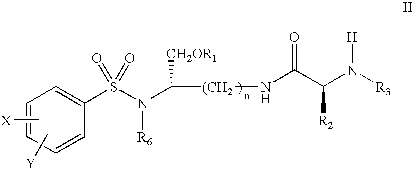

wherein n may be 3 or 4,

wherein X and Y, the same or different, may be selected from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —OCF₃, —CN, —NO₂, —NR₄R₅, —NHCOR₄, —OR₄, —SR₄, —COOR₄, —COR₄, and —CH₂OH or X and Y together may define, for example, an alkylenedioxy group which may be selected from the group consisting of a methylenedioxy group of formula —OCH₂O— and an ethylenedioxy group of formula —OCH₂CH₂O—,

wherein R₆ may be selected, for example, from the group consisting of a straight alkyl group of 1 to 6 carbon atoms, a branched alkyl group of 3 to 6 carbon atoms, a cycloalkylalkyl group having 3 to 6 carbon atoms in the cycloalkyl part thereof and 1 to 3 carbon atoms in the alkyl part thereof,

wherein R₃ may be selected, for example, from the group consisting of H, a straight alkyl group of 1 to 6 carbon atoms, a branched alkyl group of 3 to 6 carbon atoms, a cycloalkyl group of 3 to 6 carbon atoms, and a group of formula R_{3A}—CO—, where R_{3A} may be selected, for example, from the group consisting of a straight or branched alkyl group of 1 to 6 carbon atoms, a cycloalkyl group having 3 to 6 carbon atoms, a cycloalkylalkyl group having 3 to 6 carbon atoms in the cycloalkyl part thereof and 1 to 3 carbon atoms in the alkyl part thereof, an alkyloxy group of 1 to 6 carbon atoms, tetrahydro-3-furanyloxy, —CH₂OH, —CF₃, —CH₂CF₃, —CH₂CH₂CF₃, pyrrolidinyl, piperidinyl, 4-morpholinyl, CH₃O₂C—, CH₃O₂CCH₂—, Acetyl-OCH₂CH₂—, HO₂CCH₂—, 3-hydroxyphenyl, 4-hydroxyphenyl, 4-CH₃OC₆H₄CH₂—, CH₃NH—, (CH₃)₂N—, (CH₃CH₂)₂N—, (CH₃CH₂CH₂)₂N—, HOCH₂CH₂NH—, CH₃CH₂—, CH₃OCH₂CH₂O—, C₆H₅CH₂O—, 2-pyrrolyl, 2-pyridyl, 3-pyridyl, 4-pyridyl-, 2-pyrazinyl, 2-quinolyl, 3-quinolyl, 4-quinolyl, 1-isoquinolyl, 3-isoquinolyl, 2-quinoxalinyl, a phenyl group of formula

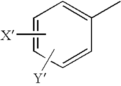

a picolyl group which may be selected from the group consisting of

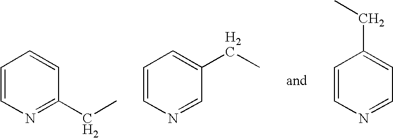

a picolyloxy group which may be selected from the group consisting of

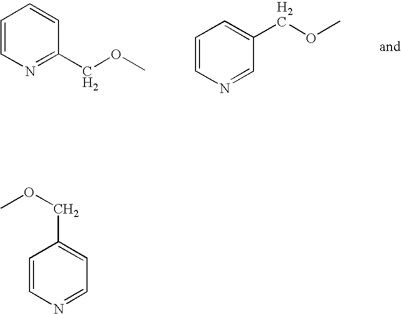

a substituted pyridyl group which may be selected from the group consisting of

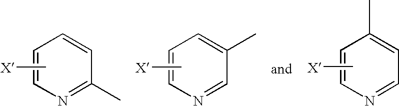

a group of formula

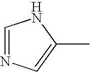

wherein X′ and Y′, the same or different, may be selected from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —NO₂, —NR₄R₅, —NHCOR₄, —OR₄, —SR₄, —COOR₄, —COR₄ and —CH₂OH,

wherein R₄ and R₅, the same or different, may be selected from the group consisting of H, a straight alkyl group of 1 to 6 carbon atoms, a branched alkyl group of 3 to 6 carbon atoms, and a cycloalkyl group of 3 to 6 carbon atoms,

wherein R₂ may be selected from the group consisting of a diphenylmethyl group of formula IV

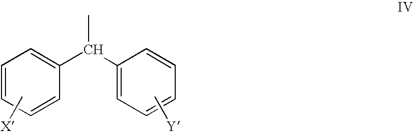

a naphthyl-1-CH₂— group of formula V

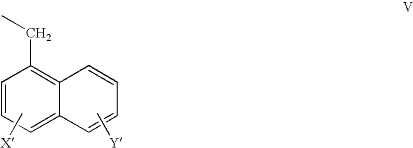

a naphthyl-2-CH₂— group of formula VI

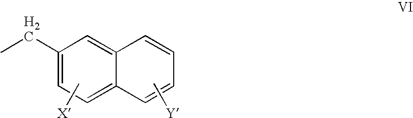

a biphenylmethyl group of formula VII

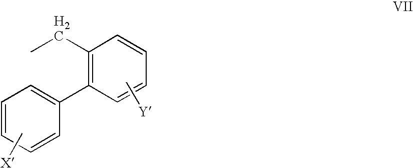

and an anthryl-9-CH₂— group of formula VIII

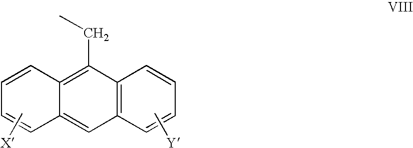

wherein R₁ may be H or a physiologically cleavable unit, whereby upon physiological conditions (in vivo) the compound may be converted into an active protease inhibitor.

In accordance with the present invention, R₁ may be selected, for example, from the group consisting of H, (HO)₂P(O) and (MO)₂P(O) (wherein M may be an alkali metal or alkaline earth metal), and a group of formula R_{1A}—CO—, where R_{1A} may be selected, for example, from the group consisting of a straight or branched alkyl group of 1 to 6 carbon atoms, a cycloalkyl group having 3 to 6 carbon atoms, a cycloalkylalkyl group having 3 to 6 carbon atoms in the cycloalkyl part thereof and 1 to 3 carbon atoms in the alkyl part thereof, an alkyloxy group of 1 to 6 carbon atoms, —CH₂OH, CH₃O₂C—, CH₃O₂CCH₂—, Acetyl-OCH₂CH₂—, HO₂CCH₂—, 2-hydroxyphenyl, 3-hydroxyphenyl, 4-hydroxyphenyl, (CH₃)₂NCH₂—, (CH₃)₂CHCH(NH₂)—, HOCH₂CH₂NH—, CH₃OCH₂O—, CH₃OCH₂CH₂O—, 2-pyrrolyl, 2-pyridyl, 3-pyridyl, 4-pyridyl, 1-methyl-1,4-dihydro-3-pyridyl, 2-pyrazinyl, 2-quinolyl, 3-quinolyl, 4-quinolyl, 1-isoquinolyl, 3-isoquinolyl, 2-quinoxalinyl, a phenyl group of formula

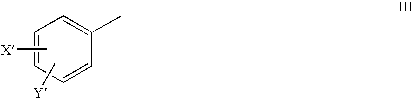

a picolyl group which may be selected from the group consisting of

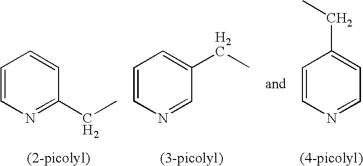

a picolyloxy group which may be selected from the group consisting of

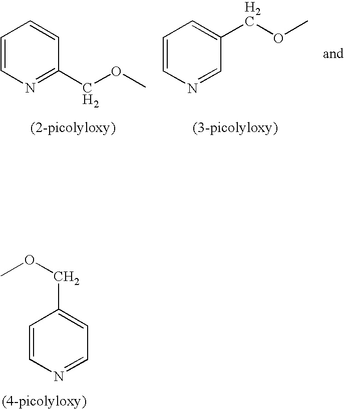

a substituted pyridyl group which may be selected from the group consisting of

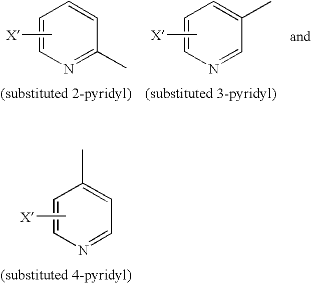

and a group of formula,

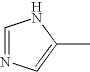

wherein X′, Y′, R₄ and R₅ are as defined herein.

In accordance with an embodiment of the present invention, pharmaceutical compositions which comprise compounds of formula II wherein R₆ may be, for example, iso-butyl and n may be 3 are encompassed herewith.

In accordance with an additional embodiment of the present invention, pharmaceutical compositions which comprise compounds of formula II wherein R₆ may be, for example, iso-butyl and n may be 4 are also encompassed herewith.

In a particular embodiment of the present invention, R₁ may be selected, for example, from the group consisting of H, (HO)₂P(O) and (NaO)₂P(O).

In another particular embodiment of the present invention, R₁ may be selected, for example, from the group consisting of CH₃CO, 3-pyridyl-CO, (CH₃)₂NCH₂CO and (CH₃)₂CHCH(NH₂)CO.

In accordance with another particular embodiment of the present invention, pharmaceutical compositions which comprise compounds of formula II wherein R₃ may be selected, for example, from the group consisting of CH₃CO, CH₃O—CO, (CH₃)₂N—CO, 3-pyridyl-CO, 4-pyridyl-CO and 4-morpholine-CO are encompassed by the present invention.

In accordance with an embodiment of the present invention, X may be 4-NH₂ and Y may be H or F.

In accordance with an embodiment of the present invention, X′ and Y′ may both be H.

In accordance with a particular embodiment of the present invention, pharmaceutical compositions which comprise compounds of formula II wherein R₂ may be selected, for example, from the group consisting of a diphenylmethyl group of formula IV, a naphthyl-1-CH₂— group of formula V, a naphthyl-2-CH₂— group of formula VI, a biphenylmethyl group of formula VII and an anthryl-9-CH₂— group of formula VIII are encompassed herewith.

For example, R₂ may, more particularly, be selected from the group consisting of a diphenylmethyl group of formula IV, a naphthyl-1-CH₂— group of formula V, and a naphthyl-2-CH₂— group of formula VI.

In a further aspect, the present invention provides pharmaceutical compositions comprising a compound of formula II, wherein R₆ is isobutyl, n is 4 and R₂ is a diphenylmethyl group of formula IV.

In accordance with an embodiment of the present invention, R₁ may be selected from the group consisting of H, (HO)₂P(O) and (NaO)₂P(O).

In accordance with a further embodiment of the present invention, R₁ may be selected from the group consisting of CH₃CO, 3-pyridyl-CO, (CH₃)₂NCH₂CO and (CH₃)₂CHCH(NH₂)CO.

In accordance with an additional embodiment of the present invention, R₃ may be selected, for example, from the group consisting of CH₃CO, CH₃O—CO, (CH₃)₂N—CO, 3-pyridyl-CO, 4-pyridyl-CO and 4-morpholine-CO.

In accordance with an embodiment of the present invention, X may be 4-NH₂ and Y may be H or F.

More particularly, in accordance with an embodiment thereof, the present invention provides a pharmaceutical composition which may comprise a compound of formula II, wherein X may be, for example, 4-NH₂, Y may be H, X′ may be H, Y′ may be H and R₃ may be CH₃O—CO.

In accordance with a particular embodiment of the present invention, R₁ may be (HO)₂P(O).

In accordance with another particular embodiment of the present invention, R₁ may be (NaO)₂P(O).

In accordance with a further particular embodiment of the present invention, R₁ may be H.

More particularly, in accordance with a further embodiment thereof, the present invention provides a pharmaceutical composition which may comprise a compound of formula II, wherein X may be 4-NH₂, Y may be 3-F, X′ may be H, Y′ may be H and R₃ may be CH₃O—CO.

In accordance with a particular embodiment of the present invention, R₁ may be (HO)₂P(O).

In accordance with another particular embodiment of the present invention, R₁ may be (NaO)₂P(O).

In accordance with a further particular embodiment of the present invention, R₁ may be H.

More particularly, in accordance with an additional embodiment thereof, the present invention provides a pharmaceutical composition which may comprise a compound of formula II, wherein X is 4-NH₂, Y is H or 3-F, X′ is H, Y′ is H and R₃ is CH₃CO.

In accordance with a particular embodiment of the present invention, R₁ may be (HO)₂P(O).

In accordance with another particular embodiment of the present invention, R₁ may be (NaO)₂P(O).

In accordance with a further particular embodiment of the present invention, R₁ may be H.

More particularly, in accordance with an embodiment thereof, the present invention further provides a pharmaceutical composition which may comprise a compound of formula II, X is 4-NH₂, Y is H or 3-F, X′ is H, Y′ is H and R₃ is 4-morpholine-CO.

In accordance with an embodiment of the present invention X may be 4-NH₂, Y may be H, X′ may be H, Y′ may be H and R₃ may be CH₃O—CO.

In accordance with a particular embodiment of the present invention R₁ may be 3-pyridyl-CO.

In accordance with another particular embodiment of the present invention R₁ may be (CH₃)₂NCH₂CO.

In accordance with yet another particular embodiment of the present invention R₁ may be (CH₃)₂CHCH(NH₂)CO.

In accordance with an additional particular embodiment of the present invention R₁ may be CH₃CO.

In an additional embodiment the present invention provides pharmaceutical compositions comprising a compound of formula II, wherein R₆ is iso-butyl, n is 4, X′ and Y′ are both H, R₂ is Naphtyl-1-CH₂—, X is 4-NH₂, Y is H, R₃ is 4-morpholine-CO and R₁ may be selected, for example, from the group consisting of H, (HO)₂P(O) and (NaO)₂P(O).

In a further embodiment, the present invention provides pharmaceutical compositions comprising a compound of formula II, wherein R₆ is iso-butyl, n is 4, X′ and Y′ are both H, R₂ is Naphtyl-2-CH₂—, X is 4-NH₂, Y is H, R₃ is CH₃O—CO and R₁ may be selected, for example, from the group consisting of H, (HO)₂P(O) and (NaO)₂P(O).

In accordance with the present invention, the cytochrome P450 monooxigenase inhibitor may be a CYP450-3A inhibitor.

In accordance with the present invention, the cytochrome P450 monooxigenase inhibitor may be selected, for example, from the group consisting of ritonavir (RTV), ketoconazole, fluconazole, nefazodone, fluvoxamine, fluoxetine, macrolide antibiotics, sertraline sulfaphenazole and erythromycin.

Also in accordance with the present invention, the pharmaceutical composition may be administered, for example, orally.

Further in accordance with the present invention, the pharmaceutical composition may be administered, for example, twice-daily.

In accordance with an embodiment of the present invention, the ratio of (the concentration of the) compound of formula I over the (concentration of the) cytochrome P450 monooxygenase inhibitor may be between about 1 (e.g., 1:1) to about 10 (e.g., 10:1).

Also in accordance with an embodiment of the present invention, the ratio of compound of formula I over the cytochrome P450 monooxygenase inhibitor may be, for example, between about 3:1 and about 6:1.

Further exemplary embodiments of Lysine-based compounds which may be used to carry out the present invention may include, for example, a compound of formula IIa;

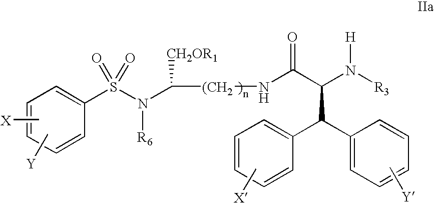

pharmaceutically acceptable salts and derivatives thereof (e.g., for example, when the compound of the present invention comprises an amino group, the pharmaceutically acceptable salt may be an ammonium salt),

wherein X and Y, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —OCF₃, —CN, —NO₂, —NR₄R₅, —NHCOR₄, —OR₄, —SR₄, —COOR₄, —COR₄, and —CH₂OH or X and Y together define an alkylenedioxy group selected from the group consisting of a methylenedioxy group of formula —OCH₂O— and an ethylenedioxy group of formula —OCH₂CH₂O—,

wherein X′ and Y′, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —NO₂, —NR₄R₅, —NHCOR₄, —OR₄, —SR₄, —COOR₄, —COR₄ and —CH₂OH,

and wherein n, R₁, R₃, R₄, R₅ and R₆ are as defined herein.

Active ingredients disclosed in U.S. Pat. No. 6,632,816 to Stranix et al, which may be used in combination with a CYP450 inhibitor in the methods, pharmaceutical compositions, kits and uses described herein, are defined by a compound of formula IIa (or a salt thereof), wherein R₁ is H and R₃, R₄, R₅, R₆, n, X, Y, X′, Y′ are as defined herein.

More particularly, the present invention provides a pharmaceutical composition which may comprise;

a) a compound of formula IIa

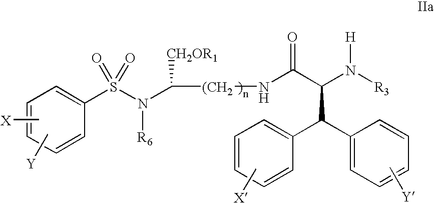

and pharmaceutically acceptable salts thereof,

wherein X and Y, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —OCF₃, —CN, —NO₂, —NR₄R₅, —NHCOR₄, —OR₄, —SR₄, —COOR₄, —COR₄, and —CH₂OH or X and Y together define an alkylenedioxy group selected from the group consisting of a methylenedioxy group of formula —OCH₂O— and an ethylenedioxy group of formula —OCH₂CH₂O—,

wherein X′ and Y′, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —NO₂, —NR₄R₅, —NHCOR₄, —OR₄, —SR₄, —COOR₄, —COR₄ and —CH₂OH,

wherein n, R₁, R₃, R₄, R₅ and R₆ are as defined herein;

b) a cytochrome P450 monooxigenase inhibitor, and;

c) a pharmaceutically acceptable carrier.

In accordance with an embodiment of the present invention, R₁ may be selected from the group consisting of H, (HO)₂P(O) and (MO)₂P(O), wherein M may be an alkali metal or alkaline earth metal and a group of formula R_{1A}—CO—, R_{1A} which may be selected from the group consisting of a straight or branched alkyl group of 1 to 6 carbon atoms, a cycloalkyl group having 3 to 6 carbon atoms, a cycloalkylalkyl group having 3 to 6 carbon atoms in the cycloalkyl part thereof and 1 to 3 carbon atoms in the alkyl part thereof, an alkyloxy group of 1 to 6 carbon atom, —CH₂OH, CH₃O₂C—, CH₃O₂CCH₂—, Acetyl-OCH₂CH₂—, HO₂CCH₂—, 2-hydroxyphenyl, 3-hydroxyphenyl, 4-hydroxyphenyl, (CH₃)₂NCH₂—, (CH₃)₂CHCH(NH₂)—, HOCH₂CH₂NH—, CH₃OCH₂O—, CH₃OCH₂CH₂O—, 2-pyrrolyl, 2-pyridyl, 3-pyridyl, 4-pyridyl, 1-methyl-1,4-dihydro-3-pyridyl, 2-pyrazinyl, 2-quinolyl, 3-quinolyl, 4-quinolyl, 1-isoquinolyl, 3-isoquinolyl, 2-quinoxalinyl, a phenyl group of formula

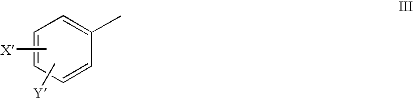

a picolyl group selected from the group consisting of

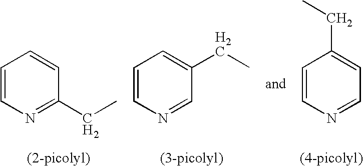

a picolyloxy group selected from the group consisting of

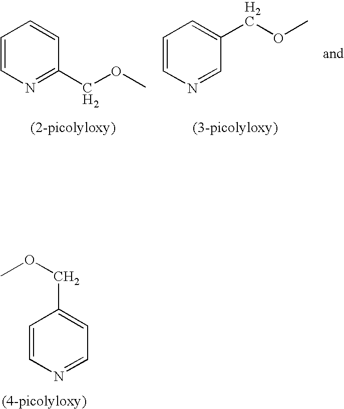

a substituted pyridyl group selected from the group consisting of

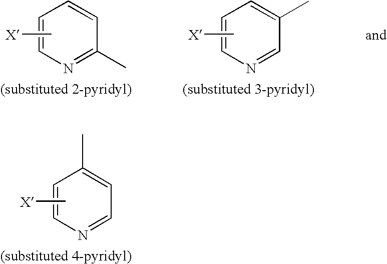

and a group of formula,

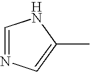

wherein X′, Y′, R₄ and R₅ are as defined herein.

In accordance with another embodiment of the present invention, R₆ may be iso-butyl.

In accordance with yet another embodiment of the present invention, n may be 4.

In accordance with a further embodiment of the present invention, R₁ may be selected from the group consisting of H, (HO)₂P(O) and (NaO)₂P(O).

Further in accordance with an embodiment of the present invention, R₁ may be selected, for example, from the group consisting of CH₃CO, 3-pyridyl-CO, (CH₃)₂NCH₂CO and (CH₃)₂CHCH(NH₂)CO.

Also in accordance with an embodiment of the present invention, R₃ may be selected from the group consisting of CH₃CO, CH₃O—CO, (CH₃)₂N—CO, 3-pyridyl-CO, 4-pyridyl-CO and 4-morpholine-CO.

In accordance with another embodiment of the present invention, R₃ may be selected from the group consisting of CH₃CO, CH₃O—CO, (CH₃)₂N—CO, 3-pyridyl-CO, 4-pyridyl-CO and 4-morpholine-CO.

In accordance with an embodiment of the present invention, X may be 4-NH₂ and Y may be H or F.

In accordance with another embodiment of the present invention, X may be 4-NH₂ and Y may be H or F.

Also in accordance with an embodiment of the present invention, X may be 4-NH₂, Y may be H or 3-F, X′ may be H, Y′ may be H and R₃ may be CH₃CO.

In accordance with another embodiment of the present invention, X may be 4-NH₂, Y may be H or 3-F, X′ may be H, Y′ may be H and R₃ may be 4-morpholine-CO.

In accordance with a particular embodiment of the present invention, X may be 4-NH₂, Y may be H, X′ may be H, Y′ may be H, R₃ may be CH₃O—CO and R₁ may be (HO)₂P(O), (NaO)₂P(O) or H.

In accordance with another particular embodiment of the present invention, X may be 4-NH₂, Y may be 3-F, X′ may be H, Y′ may be H, R₃ may be CH₃O—CO and R₁ may be (HO)₂P(O), (NaO)₂P(O) or H.

In accordance with a further particular embodiment of the present invention, X may be 4-NH₂, Y may be H or 3-F, X′ may be H, Y′ may be H, R₃ may be CH₃CO and R₁ may be (HO)₂P(O), (NaO)₂P(O) or H.

In accordance with another particular embodiment of the present invention, X may be 4-NH₂, Y may be H or 3-F, X′ may be H, Y′ may be H and R₃ may be 4-morpholine-CO.

In accordance with an additional embodiment of the present invention, X may be 4-NH₂, Y may be H, X′ may be H, Y′ may be H, R₃ may be CH₃O—CO and R₁ may be 3-pyridyl-CO, (CH₃)₂NCH₂CO, (CH₃)₂CHCH(NH₂)CO or CH₃CO.

In accordance with another embodiment of the present invention, X may be 4-NH₂, Y may be 3-F, X′ may be H, Y′ may be H, R₃ may be CH₃O—CO and R₁ may be 3-pyridyl-CO, (CH₃)₂NCH₂CO or (CH₃)₂CHCH(NH₂)CO.

Additional embodiments of Lysine-based compounds which may be used to carry out the present invention include, a compound of formula IIb

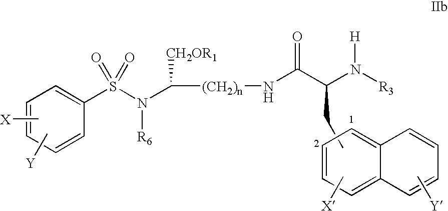

pharmaceutically acceptable salts and derivatives thereof (e.g., for example, when the compound of the present invention comprises an amino group, the pharmaceutically acceptable salt may be an ammonium salt),

wherein X and Y, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —OCF₃, —CN, —NO₂, —NR₄R₅, —NHCOR₄, —OR₄, —SR₄, —COOR₄, —COR₄, and —CH₂OH or X and Y together define an alkylenedioxy group selected from the group consisting of a methylenedioxy group of formula —OCH₂O— and an ethylenedioxy group of formula —OCH₂CH₂O—,

wherein X′ and Y′, the same or different, may be selected, for example, from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —NO₂, —NR₄R₅, —NHCOR₄, —OR₄, —SR₄, —COOR₄, —COR₄ and —CH₂OH, and wherein n, R₁, R₃, R₄, R₅ and R₆ are as defined herein.

Active ingredients disclosed in U.S. Pat. No. 6,632,816 to Stranix et al, which may be used in combination with a CYP450 inhibitor in the methods, pharmaceutical compositions, kits and uses described herein, are defined by a compound of formula IIb (or a salt thereof), wherein R₁ is H and R₃, R₄, R₅, R₆, n, X, Y, X′, Y′ are as defined herein.

The present invention therefore, more particularly provides a pharmaceutical composition which may comprise,

a) a compound of formula IIb

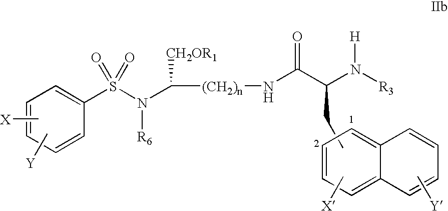

and pharmaceutically acceptable salts thereof,

wherein X and Y, the same or different, may be selected from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —OCF₃, —CN, —NO₂, —NR₄R₅, —NHCOR₄, —OR₄, —SR₄, —COOR₄, —COR₄, and —CH₂OH or X and Y together define an alkylenedioxy group which may be selected from the group consisting of a methylenedioxy group of formula —OCH₂O— and an ethylenedioxy group of formula —OCH₂CH₂O—,

wherein X′ and Y′, the same or different, may be selected from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —NO₂, —NR₄R₅, —NHCOR₄, —OR₄, —SR₄, —COOR₄, —COR₄ and —CH₂OH,

wherein n, R₁, R₃, R₄, R₅ and R₆ are as defined herein;

b) a cytochrome P450 monooxigenase inhibitor, and;

c) a pharmaceutically acceptable carrier.

In accordance with the present invention, R₁ may be selected from the group consisting of H, (HO)₂P(O) and (MO)₂P(O), wherein M may be an alkali metal or alkaline earth metal and a group of formula R_{1A}—CO—, R_{1A} which may be selected from the group consisting of a straight or branched alkyl group of 1 to 6 carbon atoms, a cycloalkyl group having 3 to 6 carbon atoms, a cycloalkylalkyl group having 3 to 6 carbon atoms in the cycloalkyl part thereof and 1 to 3 carbon atoms in the alkyl part thereof, an alkyloxy group of 1 to 6 carbon atom, —CH₂OH, CH₃O₂C—, CH₃O₂CCH₂—, Acetyl-OCH₂CH₂—, HO₂CCH₂—, 2-hydroxyphenyl, 3-hydroxyphenyl, 4-hydroxyphenyl, (CH₃)₂NCH₂—, (CH₃)₂CHCH(NH₂)—, HOCH₂CH₂NH—, CH₃OCH₂O—, CH₃OCH₂CH₂O—, 2-pyrrolyl, 2-pyridyl, 3-pyridyl, 4-pyridyl, 1-methyl-1,4-dihydro-3-pyridyl, 2-pyrazinyl, 2-quinolyl, 3-quinolyl, 4-quinolyl, 1-isoquinolyl, 3-isoquinolyl, 2-quinoxalinyl, a phenyl group of formula

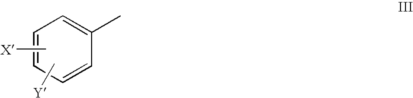

a picolyl group selected from the group consisting of

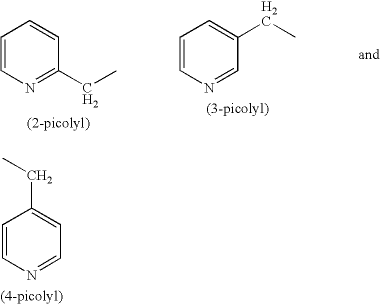

a picolyloxy group selected from the group consisting of

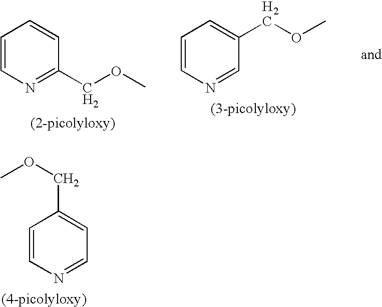

a substituted pyridyl group selected from the group consisting of

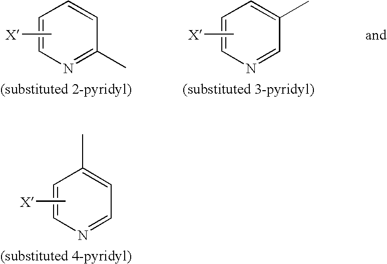

and a group of formula;

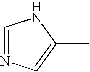

wherein X′, Y′, R₄ and R₅ are as defined herein.

In accordance with an embodiment of the present invention, R₆ may be iso-butyl.

In accordance with another embodiment of the present invention, n may be 4.

In accordance with a further embodiment of the present invention, R₁ may be selected, for example, from the group consisting of H, (HO)₂P(O) and (NaO)₂P(O).

In accordance with an additional embodiment of the present invention, R₁ may selected, for example, from the group of CH₃CO, 3-pyridyl-CO, (CH₃)₂NCH₂CO and (CH₃)₂CHCH(NH₂)CO.

In accordance with yet a further embodiment of the present invention, R₃ may be selected, for example, from the group consisting of CH₃O—CO, (CH₃)₂N—CO, 3-pyridyl-CO, 4-pyridyl-CO and 4-morpholine-CO.

In accordance with an embodiment of the present invention, R₃ may be selected, for example, from the group consisting of CH₃O—CO, (CH₃)₂N—CO, 3-pyridyl-CO, 4-pyridyl-CO and 4-morpholine-CO.

Also in accordance with an embodiment of the present invention, X may be, for example, 4-NH₂ and Y may be H or F.

Further in accordance with an embodiment of the present invention, X may be, for example, 4-NH₂ and Y may be H or F.

In accordance with a particular embodiment of the present invention, X may be 4-NH₂, Y may be H or 3-F, X′ may be H, Y′ may be H and R₃ may be CH₃O—CO.

In accordance with a further particular embodiment of the present invention, X may be 4-NH₂, Y may be H or 3-F, X′ may be H, Y′ may be H and R₃ may be CH₃CO.

In accordance with another particular embodiment of the present invention, X may be 4-NH₂, Y may be H or 3-F, X′ may be H, Y′ may be H and R₃ may be 4-morpholine-CO.

In accordance with an embodiment of the present invention the naphthyl group may be, for example, a naphthyl-2-CH₂ group, Y may be H and R₁ may be (HO)₂P(O).

In accordance with another embodiment of the present invention the naphthyl group may be, for example, a naphthyl-1-CH₂ group. Y may be H and R₁ may be (HO)₂P(O).

Further exemplary embodiments of compounds which may be used in the present invention, include, for example, a compound of formula llc

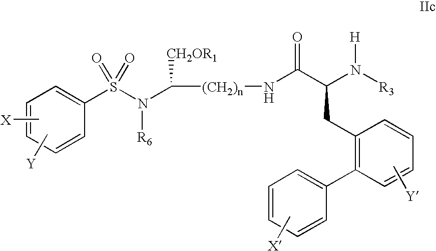

pharmaceutically acceptable salts and derivatives thereof (e.g., for example, when the compound of the present invention comprises an amino group, the pharmaceutically acceptable salt may be an ammonium salt),

and wherein n, X, Y, X′, Y′, R₁, R₃, R₄, R₅ and R₆ are as defined herein.

Active ingredients disclosed in U.S. Pat. No. 6,632,816 to Stranix et al., which may be used in combination with a CYP450 inhibitor in the methods, pharmaceutical compositions, kits and uses described herein, are defined by a compound of formula IIc (or a salt thereof), wherein R₁ is H and R₃, R₄, R₅, R₆, n, X, Y, X′, Y′ are as defined herein.

The present invention more particularly provides a pharmaceutical composition which may comprise

a) a compound of formula IIc

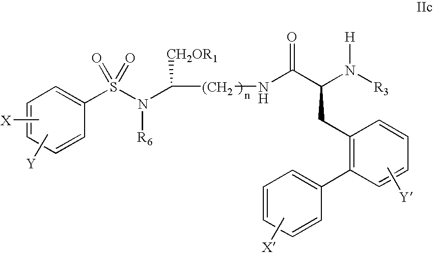

and pharmaceutically acceptable salts thereof,

wherein n, X, Y, X′, Y′, R₁, R₃, R₄, R₅ and R₆ are as defined herein;

b) a cytochrome P450 monooxigenase inhibitor, and;

c) a pharmaceutically acceptable carrier.

In accordance with an embodiment of the present invention, R₁ may be selected, for example, from the group consisting of H, (HO)₂P(O) and (MO)₂P(O), wherein M may be an alkali metal or alkaline earth metal and a group of formula R_{1A}-CO—, R_{1A} which may be selected from the group consisting of a straight or branched alkyl group of 1 to 6 carbon atoms, a cycloalkyl group having 3 to 6 carbon atoms, a cycloalkylalkyl group having 3 to 6 carbon atoms in the cycloalkyl part thereof and 1 to 3 carbon atoms in the alkyl part thereof, an alkyloxy group of 1 to 6 carbon atom, —CH₂OH, CH₃O₂C—, CH₃O₂CCH₂—, Acetyl-OCH₂CH₂—, HO₂CCH₂—, 2-hydroxyphenyl, 3-hydroxyphenyl, 4-hydroxyphenyl, (CH₃)₂NCH₂—, (CH₃)₂CHCH(NH₂)—, HOCH₂CH₂NH—, CH₃OCH₂O—, CH₃OCH₂CH₂O—, 2-pyrrolyl, 2-pyridyl, 3-pyridyl, 4-pyridyl, 1-methyl-1,4-dihydro-3-pyridyl, 2-pyrazinyl, 2-quinolyl, 3-quinolyl, 4-quinolyl, 1-isoquinolyl, 3-isoquinolyl, 2-quinoxalinyl, a phenyl group of formula

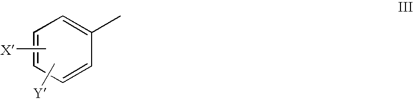

a picolyl group selected from the group consisting of

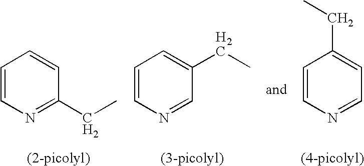

a picolyloxy group selected from the group consisting of

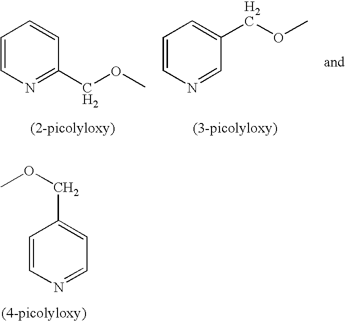

a substituted pyridyl group selected from the group consisting of

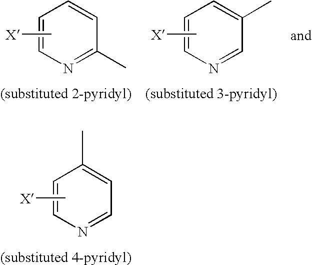

and a group of formula,

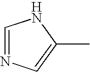

wherein X′, Y′, R₄ and R₅ are as defined herein.

In accordance with another embodiment of the present invention, R₆ may be iso-butyl.

In accordance with yet another embodiment of the present invention, n may be 4.

In accordance with a further embodiment of the present invention, R₁ may be, for example, H(HO)₂P(O) or (NaO)₂P(O).

In accordance with an additional embodiment of the present invention, R₁ may be selected, for example, from the group consisting of CH₃CO, 3-pyridyl-CO, (CH₃)₂NCH₂CO and (CH₃)₂CHCH(NH₂)CO.

Other compounds which may be used to carry out the present invention may include, for example, a compound of formula IIA;

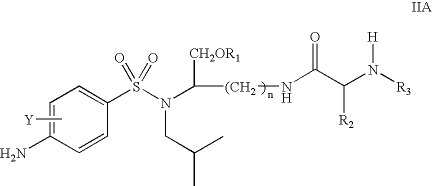

wherein Y, n, R₁, R₂, R₃, X′ and Y′ are as defined herein.

In accordance with the present invention, R₁ may be, for example, (HO)₂P(O) or (NaO)₂P(O). Further in accordance with the present invention, n may be 4. Y may be, for example, H. R₃ may be, for example, CH₃O—CO. R₂ may be, for example, a diphenylmethyl group of formula IV, where X′ and Y′ may be, for example H,

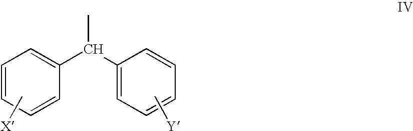

Active ingredients disclosed in U.S. Pat. No. 6,632,816 to Stranix et al, which may be used in combination with a CYP450 inhibitor in the methods, pharmaceutical compositions, kits and uses described herein, are defined by a compound of formula IIA (or a salt thereof), wherein R₁ is H and R₂, R₃, R₄, R₅, n, X, Y, X′, Y′ are as defined herein.

Compounds of formula IIA′ as well as pharmaceutically acceptable salts and derivatives thereof may be used to carry out the present invention,

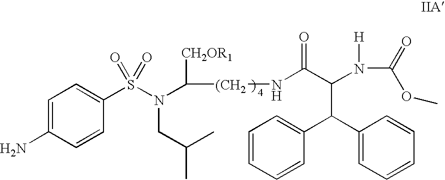

such as, for example, compound of formula IIA′ wherein R₁ is (HO)₂P(O) or, compound of formula IIA′ wherein R₁ is (NaO)₂P(O).

Active ingredients disclosed in U.S. Pat. No. 6,632,816 to Stranix et al, which may be used in combination with a CYP450 inhibitor in the methods, pharmaceutical compositions, kits and uses described herein, are defined by a compound of formula IIA′ (or a salt thereof), wherein R₁ is H are as defined herein.

For example, pharmaceutical compositions, methods, uses and kits encompassed by the present invention may comprise one or more of the following compounds and combination thereof;

a compound of formula IIa wherein n is 4, R₁ is (HO)₂P(O), X is 4-NH₂, Y is H, X′is H, Y′ is H, R₆ is iso-butyl and R₃ is CH₃O—CO,

a compound of formula IIa wherein n is 4, R₁ is (NaO)₂P(O), X is 4-NH₂, Y is H, X′ is H, Y′ is H, R₆ is iso-butyl and R₃ is CH₃O—CO,

a compound of formula IIa wherein n is 4, R₁ is (HO)₂P(O), X is 4-NH₂, Y is H, X′ is H, Y′ is H, R₆ is iso-butyl and R₃ is CH₃CO,

a compound of formula IIa wherein n is 4, R₁ is (HO)₂P(O), X is 4-NH₂, Y is 3-F, X′ is H, Y′ is H, R₆ is iso-butyl and R₃ is CH₃O—CO,

a compound of formula IIa wherein n is 4, R₁ is CH₃CO, X is 4-NH₂, Y is H, X′ is H, Y′ is H, R₆ is iso-butyl and R₃ is CH₃O—CO,

a compound of formula IIa wherein n is 4, R₁ is 3-pyridyl-CO, X is 4-NH₂, Y is H, X′ is H, Y′ is H, R₆ is iso-butyl and R₃ is CH₃O—CO,

a compound of formula IIa wherein n is 4, R₁ is (CH₃)₂NCH₂CO, X is 4-NH₂, Y is H, X′ is H, Y′ is H, R₆ is iso-butyl and R₃ is CH₃O—CO,

a compound of formula IIa wherein n is 4, R₁ is (CH₃)₂CHCH(NH₂)CO, X is 4-NH₂, Y is H, X′ is H, Y′ is H, R₆ is iso-butyl and R₃ is CH₃O—CO,

a compound of formula IIb wherein n is 4, R₁ is (HO)₂P(O), X is 4-NH₂, Y is H, X′ is H, Y′ is H, R₆ is iso-butyl and R₃ is CH₃O—CO and wherein the naphthyl group is a naphthyl-2-CH₂ group,

a compound of formula IIb wherein n is 4, R₁ is (HO)₂P(O), X is 4-NH₂, Y is H, X′ is H, Y′ is H, R₆ is iso-butyl and R₃ is 4-morpholine-CO and wherein the naphthyl group is a naphthyl-1-CH₂ group, or

a combination of any of the above mentioned compounds.

Any compound which is a precursor of an active ingredient described herein may be used to carry out the present invention and is also encompassed by the present invention. For example, compounds being able to release or generate (either in vivo or in vitro) an active ingredient of the following formula;

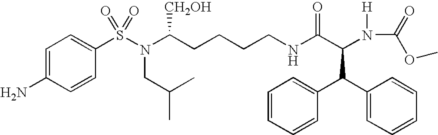

are encompassed by the present invention.

The above identified active ingredient is identified herein as PL-100 and it is to be understood herein that any precursor able to release or generate the above mentioned exemplary compound either in vivo or in vitro is encompassed by the present invention and may be used to carry out methods, pharmaceutical compositions, kits and uses described herein.

In addition, compounds which able to release (either in vivo or in vitro) an active ingredient of the following formula;

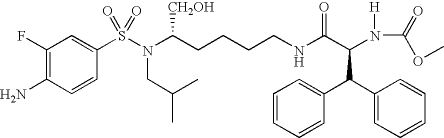

may be used to carry out the present invention and are therefore encompassed by the present invention.

The above identified active ingredient is identified herein as PL-337 and it is to be understood herein that any precursor able to release or generate the above mentioned exemplary compound either in vivo or in vitro may be used to carry out the present invention and is encompassed by the present invention.

The present invention also relates in an additional aspect thereof, to the use of at least one compound of formula I

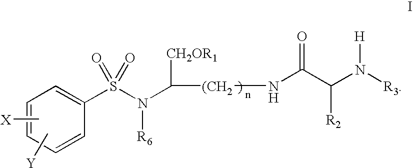

or a pharmaceutically acceptable salts thereof and a cytochrome P450 monooxigenase inhibitor (e.g., a compound of formula I in combination with a cytochrome P450 monooxigenase inhibitor) in the manufacture of a drug for the treatment or prevention of an HIV infection or for treatment or prevention of AIDS, wherein n, X, Y, X′, Y′, R₁, R₂, R₃, R₄, R₅ and R₆ are as defined herein.

In accordance with an embodiment of the present invention, the cytochrome P450 monooxigenase inhibitor may be selected from the group consisting of ritonavir (RTV), ketoconazole, fluconazole, nefazodone, fluvoxamine, fluoxetine, macrolide antibiotics, sertraline sulfaphenazole and erythromycin.

The present invention also provides in an additional aspect thereof to a kit for treating or preventing an HIV infection or for treating or preventing AIDS, the kit may comprise, for example, a) a first container which may contain a compound of formula I (e.g., in the form of a pharmaceutical composition comprising a compound of formula I or a pharmaceutically acceptable salts thereof and a pharmaceutically acceptable carrier);

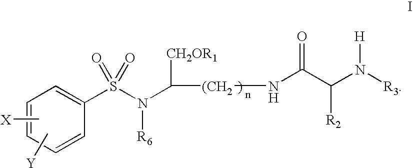

wherein n, X, Y, X′, Y′, R₁, R₂, R₃, R₄, R₅ and R₆ are as defined in claim 1, and a second container containing a cytochrome P450 monooxigenase inhibitor (e.g., in the form of a pharmaceutical composition comprising a CYP450 inhibitor and a pharmaceutically acceptable carrier) or;

b) a container comprising both the compound of formula I and the CYP450 inhibitor (e.g., in the form of a pharmaceutical composition comprising a compound of formula I, a CYP450 inhibitor and a pharmaceutically acceptable carrier).

In yet an additional aspect, the present invention provides a method of treating or preventing an HIV infection or of treating or preventing AIDS, the method may comprise administering a pharmaceutical composition as defined in claim 1 to a mammal in need thereof.

In accordance with an embodiment of the present invention, administration of the pharmaceutical composition may be performed, for example, orally.

In accordance with an embodiment of the present invention the pharmaceutical composition may be administered, for example, twice-daily.

In another aspect, the present invention provides a method of treating or preventing an HIV infection or of treating or preventing AIDS, the method may comprise administering (e.g., co-administering)

a) a compound of formula I

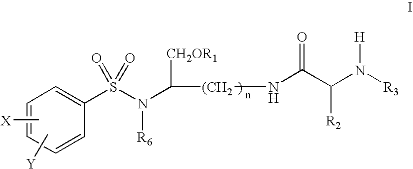

or a pharmaceutically acceptable salts thereof, wherein n, X, Y, X′, Y′, R₁, R₂, R₃, R₄, R₅ and R₆ are as defined herein, and;

b) one or more CYP450 inhibitor in an amount which is sufficient to reduce the metabolism of the compound of formula I.

Further in accordance with the present invention, administration of the compound of formula I and the CYP450 inhibitor may be performed, for example, separately, simultaneously or sequentially.

In accordance with a particular embodiment of the present invention, administration of the compound of formula I and the CYP450 inhibitor may be performed, for example, by administering a) a first pharmaceutical composition which may comprise a compound of formula I and a pharmaceutically acceptable carrier and b) a second pharmaceutical composition which may comprise a CYP450 inhibitor and a pharmaceutically acceptable carrier.

Further in accordance with the present invention, administration of the compound of formula I and the CYP450 inhibitor may be performed, for example, by administering a single pharmaceutical composition which may comprise a compound of formula I, a CYP450 inhibitor and a pharmaceutically acceptable carrier.

The present invention also relates in a further aspect thereof to a method for improving the pharmacokinetics of a compound of formula I as defined herein, the method may comprise administering to a human in need thereof, the compound of formula I and (e.g., in combination with) an amount of a CYP450 inhibitor effective to inhibit cytochrome P450 monooxygenase.

In yet a further aspect, the present invention relates to a method of inhibiting (e.g., reducing replication of) an HIV (e.g., one or more isolate, one or more strain) having a reduced susceptibility to a protease inhibitor other than the protease inhibitor defined by formula I, the method may comprise administering a compound of formula I alone or in combination with a CYP450 inhibitor to an individual in need thereof.

In accordance with an embodiment of the present invention, the HIV may be an HIV-1.

Also in accordance with an embodiment of the present invention, the HIV may be one that has a reduced susceptibility to one or more of a protease inhibitor which may be selected, for example, from the group consisting of Atazanavir, Amprenavir, Indinavir, Lopinavir, Nelfinavir, Ritonavir and Saquinavir.

Further in accordance with an embodiment of the present invention, the HIV-1 may be one that possesses an aspartyl protease having one or more mutations (a mutation conferring a resistance to one or more of protease inhibitor mentioned herein.

The compounds listed herein are exemplary embodiments of compounds which may be used to carry out the present invention and it is to be understood that the present invention is not restricted to these compounds only.

The term “pharmaceutically effective amount” refers to an amount effective in treating, preventing or reducing the risk or probability of HIV infection or of reducing HIV burden. The term “pharmaceutically effective amount” also refers to an amount effective in treating, preventing or reducing the risk or probability of developing acquired immunodeficiency syndrome (AIDS), for delaying the apparition of AIDS, or reducing AIDS symptoms. It is also to be understood herein that a “pharmaceutically effective amount” may be construed as an amount giving a desired therapeutic effect, either taken into a single or multiple doses or in any dosage or route or taken alone or in combination with other therapeutic agents. In the case of the present invention, a “pharmaceutically effective amount” may be understood as an amount having an inhibitory effect (partial or complete) on HIV (HIV-1 and HIV-2 as well as related viruses (e.g., HTLV-I and HTLV-II, and simian immunodeficiency virus (SIV))) infection cycle (e.g., inhibition of replication, reinfection, maturation, budding etc.) and on any organism which rely on aspartyl proteases for its life cycle. An inhibitory effect is to be understood herein as an effect such as a reduction in the capacity of an organism (e.g. HIV) to reproduce itself (replicate), to re-infect surrounding cells, etc, or even a complete inhibition (or elimination) of an organism.

The terms “HIV protease” and “HIV aspartyl protease” are used interchangeably and includes, for example, the aspartyl protease encoded by the human immunodeficiency virus type 1 or 2.

The term “prophylactically effective amount” refers to an amount effective in preventing or reducing the risk or probability of HIV infection in a patient. As used herein, the term “patient” or “individual” refers to a mammal, including for example, a human.

The terms “pharmaceutically acceptable carrier”, “pharmaceutically acceptable adjuvant” and “physiologically acceptable vehicle” refer to a non-toxic carrier or adjuvant that may be administered to a patient, together with one or more compounds of the present invention, and which does not destroy the pharmacological activity thereof.

The term “precursor” refers to a compound, such as a Lysine-based compound which is able to be converted into an active ingredient in vitro or in vivo. For example, the compound PL-461 is a precursor of compound PL-100 as when administered to an individual, PL-461 is converted into PL-100 in vivo (e.g., under physiological conditions).

The term “derivative” refers to a compound which has been chemically synthesized from an original compound. For example, when considering the chemical synthesis of PL-461, PL-461 is a derivative of PL-100.

The term “consisting essentially of” means that the pharmaceutical composition includes the specified materials and may include other material that does not materially affect the basic characteristics of the pharmaceutical composition.

Pharmaceutically acceptable derivatives of the compounds of formula I (such as compounds of formulae I, II, IIa, IIb, IIc, IIA and IIA′) and where applicable pharmaceutically acceptable salts thereof such as, for example, ammonium salts are described herein. A “pharmaceutically acceptable derivative” means any pharmaceutically acceptable salt, ester, or salt of such ester, of a compound of this invention or any other compound which, upon administration to a recipient (a mammal), is capable of providing (directly or indirectly) a an active compound or an antivirally active metabolite or residue thereof.

It is to be understood herein that a “straight alkyl group of 1 to 6 carbon atoms” includes for example, methyl, ethyl, propyl, butyl, pentyl, hexyl.

It is to be understood herein that a “branched alkyl group of 3 to 6 carbon atoms” includes for example, without limitation, iso-butyl, tert-butyl, 2-pentyl, 3-pentyl, etc.

It is to be understood herein, that a “cycloalkyl group having 3 to 6 carbon” includes for example, without limitation, cyclopropyl, cyclobutyl, cyclopentyl, cyclocyclohexyl (i.e., C₆H₁₁).

Salts derived from appropriate bases include alkali metal (e.g., sodium), alkaline earth metal (e.g., magnesium), ammonium and N—(C₁₋₄ alkyl)₄⁺ salts.

The compounds described herein contain one or more asymmetric carbon atoms and thus may occur as racemates and racemic mixtures, single enantiomer, diastereomeric mixtures and individual diastereoisomers. All such isomeric forms of these compounds are expressly included in the present invention. Each stereogenic carbon may be of the R or S configuration.

Pharmaceutically acceptable salts of the compounds described herein include those derived from pharmaceutically acceptable inorganic and organic acids and bases. Examples of such acid salts include: acetate, adipate, alginate, aspartate, benzoate, benzenesulfonate, bisulfate, butyrate, citrate, camphorate, camphorsulfonate, cyclopentanepropionate, digluconate, dodecylhydrogensulfate, dodecylsulfate, ethanesulfonate, formate, fumarate, glucoheptanoate, glycerophosphate, glycollate, hemisulfate, heptanoate, hexanoate, hydrochloride, hydrobromide, hydroiodide, 2-hydroxyethanesulfonate, lactate, maleate, malonate, methanesulfonate, 2-naphthylsulfonate, nicotinate, nitrate, oxalate, pamoate, pectinate, perchlorate, persulfate, 3-phenylpropionate, phosphate, picrate, pivalate, propionate, salicylate, succinate, sulfate, tartrate, thiocyanate, tosylate, and undecanoate.

Compounds which are encompassed by the present invention also envisions the quaternization of any basic nitrogen containing groups of the compounds disclosed herein. The basic nitrogen may be quaternized with any agents known to those of ordinary skill in the art including, for example, lower alkyl halides, such as methyl, ethyl, propyl and butyl chlorides, bromides and iodides; dialkyl sulfates including dimethyl, diethyl, dibutyl and diamyl sulfates; long chain halides such as decyl, lauryl, myristyl and stearyl chlorides, bromides and iodides, and aralkyl halides including benzyl and phenethyl bromides. Water or oil-soluble or dispersible products may be obtained by such quaternization.

It is to be understood herein, that if a “range” or “group of substances” is mentioned with respect to a particular characteristic (e.g., temperature, concentration, time and the like) of the present invention, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example,
with respect to the number of carbon atoms, the mention of the range of 1 to 6 carbon atoms is to be understood herein as incorporating each and every individual number of carbon atoms as well as sub-ranges such as, for example, 1 carbon atoms, 3 carbon atoms, 4 to 6 carbon atoms, etc.
with respect to reaction time, a time of 1 minute or more is to be understood as specifically incorporating herein each and every individual time, as well as sub-range, above 1 minute, such as for example 1 minute, 3 to 15 minutes, 1 minute to 20 hours, 1 to 3 hours, 16 hours, 3 hours to 20 hours etc.;
and similarly with respect to other parameters such as concentrations, elements, etc. . . .

It is in particular to be understood herein that the compound formulae each include each and every individual compound described thereby as well as each and every possible class or sub-group or sub-class of compounds whether such class or sub-class is defined as positively including particular compounds, as excluding particular compounds or a combination thereof; for example an exclusionary definition for the formula (e.g. I) may read as follows: “provided that when one of A and B is —COOH and the other is H, —COOH may not occupy the 4′ position”.

It is also to be understood herein that “g” or “gm” is a reference to the gram weight unit and “C”, or “° C.” is a reference to the Celsius temperature unit.

The compounds described herein may easily be prepared using conventional techniques from readily available starting materials. The detailed descriptions of these approaches are presented, for example, in schemes 1 to 5 discussed below.

Scheme 1 illustrates a generic example for the preparation of the phosphate monoester III derived from a primary alcohol (see I), a compound of HIV protease inhibitors (see example 1 (step G and H) in the experimental portion of this document for a specific example of this synthesis).

Note:

a) R₂ and R₃ are as defined herein.

The synthesis of phosphate monoester III may use a HIV aspartyl protease inhibitor (I, see U.S. Pat. No. 6,632,816) as the starting material. The diethyl phosphotriester II was obtained in good yield upon treatment with diethyl chlorophosphate and sodium hydride in a mixture of tetrahydrofuran and triethylphosphate. Then, addition of trimethysilyl bromide in dichloromethane (DCM) gave compound III in good to excellent yields.

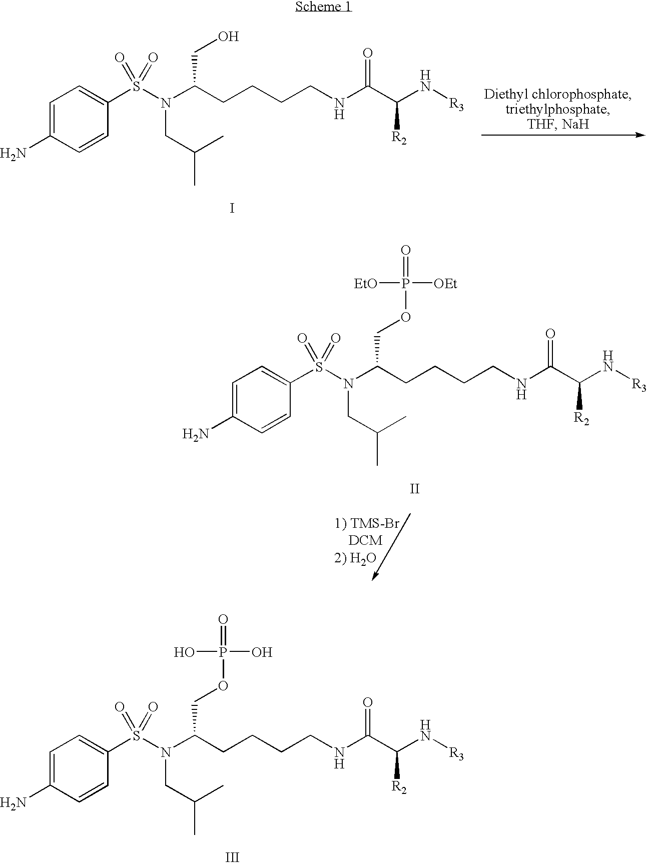

Scheme 1A represents another generic example for the preparation of the phosphate monoester IIIA derived from a primary alcohol (see IA), a compound of HIV protease inhibitors.

Note:

a) n, X, Y, R₂, R₃ and R₆ are as defined herein.

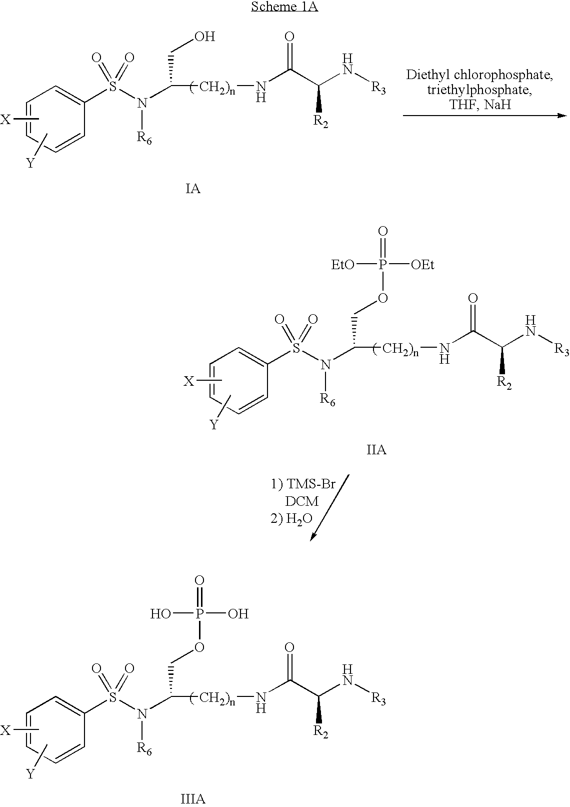

The synthesis of phosphate monoester IIIA is performed as described for the preparation of III (scheme 1).

Scheme 2 illustrates a generic example for the preparation of the phosphate monoester III, a compound of HIV protease inhibitors, with a different approach starting from (3S)-3-isobutylamino-azepan-2-one (IV).

Note:

a) R₂ and R₃ are as defined herein.

As shown in scheme 2, the phosphate monoester derivative III was obtained from (3S)-3-isobutylamino-azepan-2-one (IV) in a seven-step reaction sequence. Initially, (2S)-3-isobutylamino-azepan-2-one (IV) was sulfonated with 4-acetamidobenzenesulfonyl chloride in the presence of triethylamine in dichloromethane to give compound V in excellent yields. The derivative VI was obtained quantitatively upon treatment of V with di-tert-butyl pyrocarbonate and DMAP in acetonitrile. The reductive ring opening with sodium borohydride in ethanol lead to key intermediates VI in good yield. The diethyl phosphotriester VIII was obtained in good yield upon treatment with diethyl chlorophosphate and sodium hydride in a mixture of tetrahydrofuran and triethylphosphate. The Boc protective groups were removed upon treatment with HCl in ethanol to give compound IX quantitatively (T. W. Greene and P. G. M. Wuts, Protective groups in Organic Synthesis, 3^{rd} Edition, John Wiley & Sons, Inc. 1999). Then, coupling of the free amino group present on intermediate IX with a variety of synthetic amino acid in the presence of 1-hydroxybenzotriazole (HOBt) and 1-[3-(dimethylamino)propyl]-3-ethylcarbodiimide hydrochloride (EDAC) led to derivative II in good to excellent yields. Finally, addition of trimethysilyl bromide in dichloromethane (DCM) gave compound III in good to excellent yields.

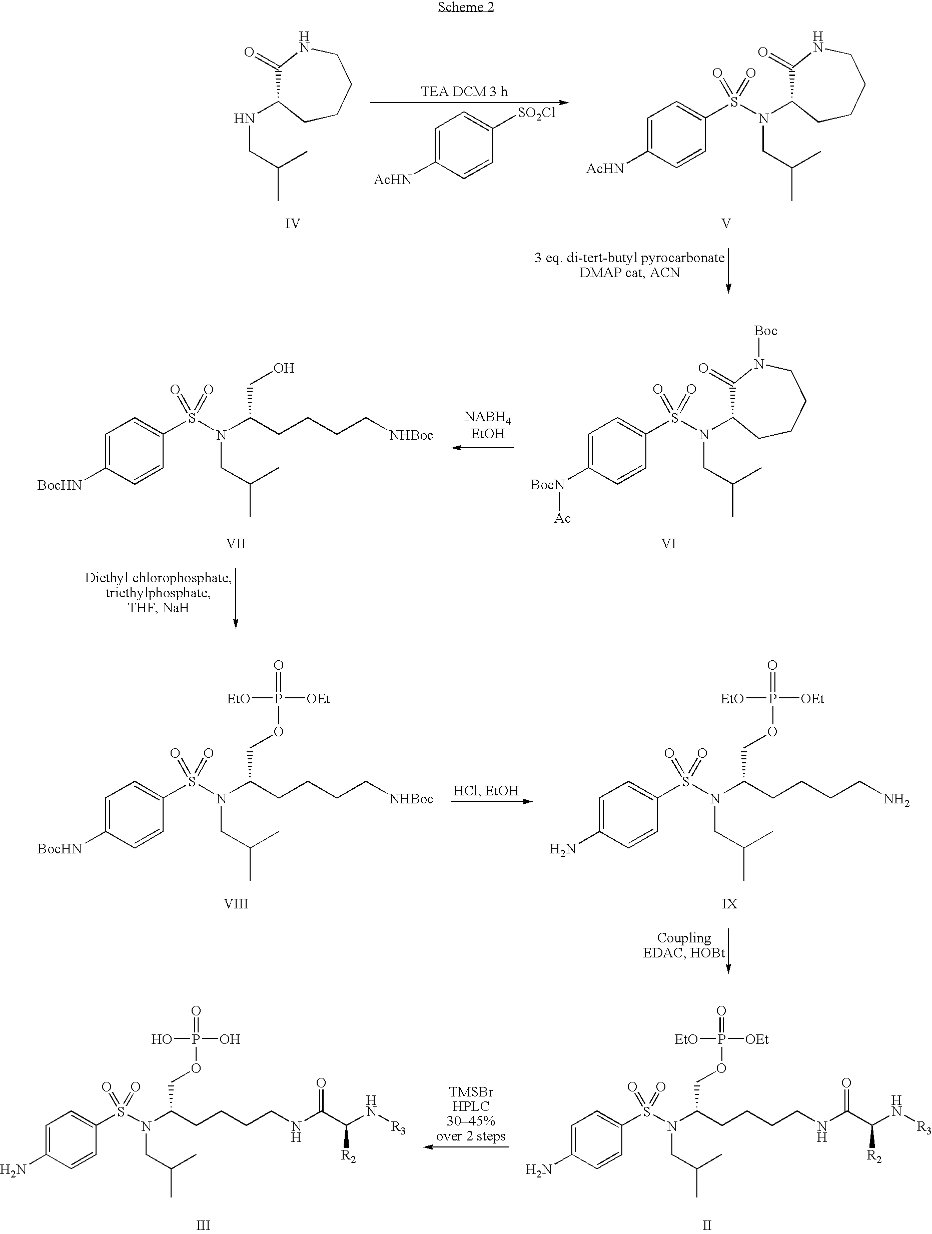

Scheme 3 presents the transformation of a diphenylmethyl derivative; (1S,5S)-(1-{5-[(4-amino-benzenesulfonyl)-isobutyl-amino]-6-hydroxy-hexylcarbamoyl}-2,2-diphenyl-ethyl)-carbamic acid methyl ester (PL-100) into its fluorinated phosphate monoester sodium salt analog XI. This reaction sequence may be used to produce any other similar compounds (compounds) made of unsubstituted (or substituted) diphenylmethyl, 1-naphthyl, 2-naphthyl, biphenyl and 9-anthryl groups described herein.

Thus, the treatment of PL-100 with Selectfluor™ in acetonitrile gave derivative X in 38% yield. The introduction of the phosphate monoester group was performed as described previously in scheme 1 and 2. First, the diethyl phosphotriester intermediate was obtained in good yield upon treatment with diethyl chlorophosphate and sodium hydride in a mixture of tetrahydrofuran and triethylphosphate. Secondly, addition of trimethysilyl bromide in dichloromethane (DCM) gave the phosphate monoester compound in good to excellent yields. The final product XI was easily obtained upon treatment of the phosphate monoester with a solution of sodium hydroxide with good yields.

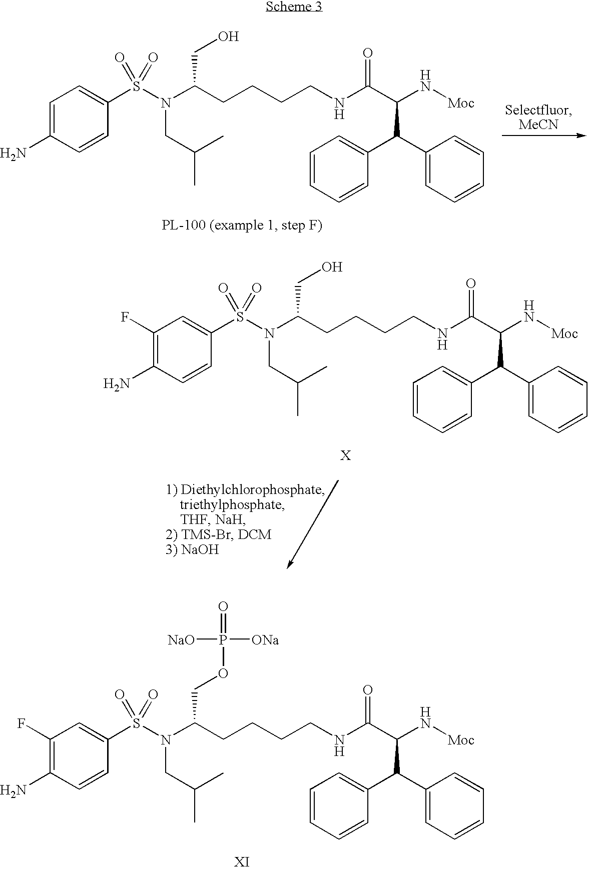

Scheme 4 illustrates a generic example for the transformation of a phosphotriester II into its fluorinated analog XIII in a two-step reaction sequence. This generic example represents a second approach for the synthesis of fluorinated compounds described herein. In this case, the fluorine atom is added to the phosphotriester II instead of the primary alcohol derivative of general formula or, more specifically, PL-100 as shown on scheme 3. This alternate reaction sequence may be used to produce any other similar compounds made of unsubstituted (or substituted) diphenylmethyl, 1-naphthyl, 2-naphthyl, biphenyl and 9-anthryl groups described herein.

Note:

a) R₂ and R₃ are as defined herein.

Briefly, treatment of derivative II with Selectfluor™ in acetonitrile gave derivative XII in good yields. Then, addition of trimethysilyl bromide in dichloromethane (DCM) gave the phosphate monoester compound XIII in good to excellent yields. If desired, the final product XIII may be easily transformed into the phosphate monoester sodium salt analog as described before in scheme 3.

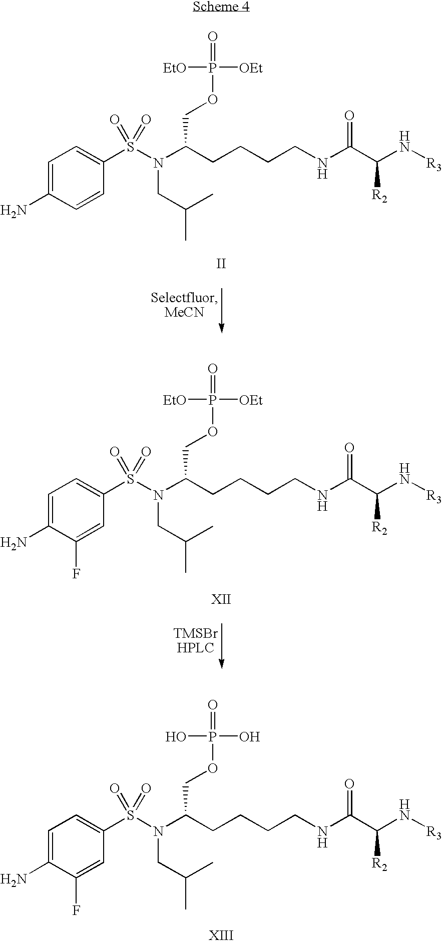

Scheme 5 illustrates exemplary synthesis of various ester compounds XVI described herein. The ester compounds are known to be easily cleaved in vivo by esterase enzymes and, as a result, may release the active ingredient. In this scheme R₂ is set as a diphenylmethyl group. However, this reaction sequence may be used to produce any other similar compounds made of unsubstituted (or substituted) diphenylmethyl, 1-naphthyl, 2-naphthyl, biphenyl and 9-anthryl groups described herein.

Note:

a) R_{1A} represents the “residue” of the acid molecule that is linked to the free primary alcohol group present on intermediate XV and is as defined herein.

The compounds XVI are generally obtained in a three-step reaction sequence in high yields. Esterification of (1S)-{4-[(5-tert-butoxycarbonylamino-1-hydroxymethyl-pentyl)-isobutyl-sulfamoyl]-phenyl}-carbamic acid tert-butyl ester (VII) with a variety of acid in the presence of 1-hydroxybenzotriazole (HOBt) and 1-[3-(dimethylamino)propyl]-3-ethylcarbodiimide hydrochloride (EDAC) led to the desired esters XIV in excellent yields. The acetyl ester was obtained quantitatively using acetic anhydride in the presence of N,N-dimethylaminopyridine (DMAP) in dichloromethane (DCM). Cleavage of the Boc protective group was achieved quantitatively upon treatment with trifluoroacetic acid (TFA) in DCM. A second coupling with (2S)-2-methoxycarbonylamino-3,3-diphenyl-propionic acid is performed on the primary amino group of intermediate XV with HOBt and EDAC to give the desired compounds XVI in good to excellent yields. If necessary, catalytic hydrogenation of a benzyloxycarbonyl group is performed using 10% palladium on carbon to give the final compound XVII.

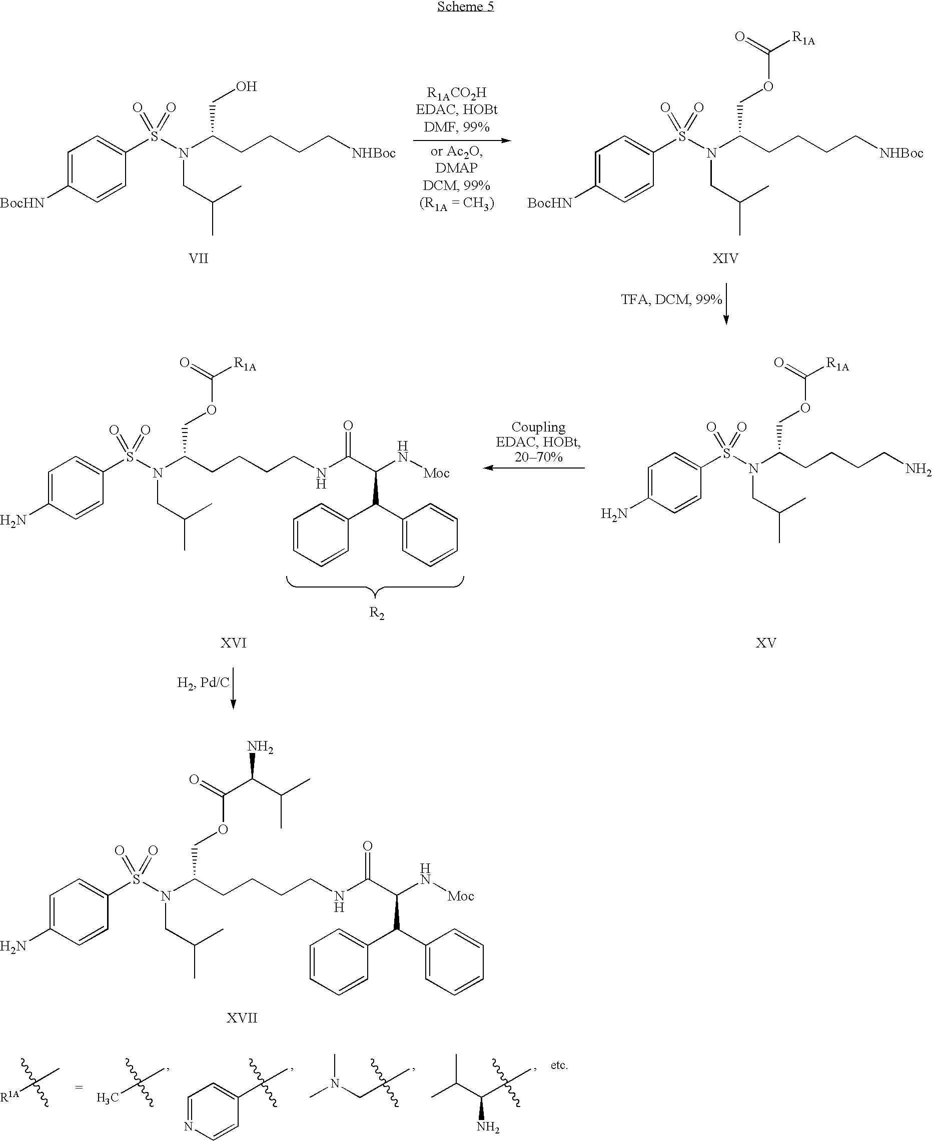

As it may be appreciated by the person skilled in the art, the above synthetic schemes are not intended to be a comprehensive list of all means by which the compound described in this application may be synthesized but only represent exemplification of synthesis methods among others. Further methods will be evident to those of ordinary skill in the art.

The compounds described herein may be modified by appending appropriate functionalities to enhance selective biological properties. Such modifications may include those which increase biological penetration into a given biological system (e.g., blood, lymphatic system, central nervous system), increase oral availability, increase solubility to allow administration by injection, alter metabolism and alter rate of excretion.

As discussed above, the Lysine-based compounds may release active ingredients which are excellent ligands for aspartyl proteases, for example, HIV-1 protease. Accordingly, these compounds are, by releasing the active ingredient, also capable of targeting and inhibiting late stage events in the replication, i.e. the processing of the viral polyproteins by HIV encoded protease. Compounds described herein advantageously inhibit the ability of the HIV-1 virus to infect immortalized human T cells over a period of days, as determined by an assay measuring the amount of extracellular p24 antigen; a specific marker of viral replication. (see, Meek et al., Nature, 343, pp. 90-92 (1990)).

In addition to their use in the prophylaxis or treatment of HIV or HTLV infection, the combination described herein may also be used as inhibitory or interruptive agents for other viruses which use aspartyl proteases, similar to HIV or HTLV aspartyl proteases, in their life cycle. Such compounds inhibit the proteolytic processing of viral polyprotein precursors by inhibiting aspartyl protease. Because aspartyl protease is essential for the production of mature virions, inhibition of that processing effectively blocks the spread of virus by inhibiting the production and reproduction of infectious virions, particularly from acutely and chronically infected cells. The compounds described herein advantageously inhibit aspartyl proteases, thus blocking the ability of aspartyl proteases to catalyze the hydrolysis of peptide bonds.

The combination described herein may be employed in a conventional manner for the treatment or prevention of HIV, HTLV, and other viral infections, which involve aspartyl proteases for their life (replication) cycle. Such methods of treatment, their dosage levels and requirements may be selected by those of ordinary skill in the art from available methods and techniques. For example, combination of a compound as described herein and a CYP450 inhibitor may be associated with a pharmaceutically acceptable adjuvant for administration to a virally infected patient in a pharmaceutically acceptable manner and in an amount effective to lessen the severity of the viral infection.

Alternatively, the combination described herein may be used in vaccines and methods for protecting individuals against viral infection over an extended period of time. The combination may be employed in such vaccines either alone or together with other compounds of this invention in a manner consistent with the conventional utilization of protease inhibitors or protease inhibitors derivatives in vaccines. For example, combination of a compound as described herein with a CYP450 inhibitor may be combined with pharmaceutically acceptable adjuvants, or delivery systems conventionally employed in vaccines and administered in prophylactically effective amounts to protect individuals over an extended period of time against viral infections, such as HIV infection. As such, the novel compounds of the present invention (upon cleavage of a physiologically cleavable unit) may be administered in combination with a CYP450 inhibitor as therapeutic agents for treating or preventing viral infections, including HIV infection, in a mammal and including AIDS treatment or prevention.

The combination of compounds described herein with a CYP450 inhibitor may be administered to a healthy or HIV-infected patient (before or after the onset of AIDS symptoms) either as a single agent or in combination with other antiviral agents which interfere with the replication cycle of HIV. By administering the compounds of this invention with other antiviral agents which target different events in the viral life cycle, the therapeutic effect of these compounds is potentiated. For instance, the co-administered antiviral agent may be one which targets early events in the viral life cycle, such as attachment to the cell receptor and cell entry, reverse transcription and viral DNA integration into cellular DNA. Antiviral agents targeting such early life cycle events include among others polysulfated polysaccharides, sT4 (soluble CD4) and other compounds which block binding of virus to CD4 receptors on CD4 bearing T-lymphocytes and other CD4(+) cells, or inhibit fusion of the viral envelope with the cytoplasmic membrane, and didanosine (ddI), zalcitabine (ddC), stavudine (d4T), zidovudine (AZT) and lamivudine (3TC) which inhibit reverse transcription. For example another protease inhibitor may be used with compounds of the present invention. Other anti-retroviral and antiviral drugs may also be co-administered with the compounds of this invention to provide therapeutic treatment for substantially reducing or eliminating viral infectivity and the symptoms associated therewith. Examples of other antiviral agents include ganciclovir, dideoxycytidine, trisodium phosphonoformate, eflomithine, ribavirin, acyclovir, alpha interferon and trimenotrexate. Additionally, other types of drugs may be used to potentiate the effect of the compounds of this invention, such as viral uncoating inhibitors, inhibitors of Tat or Rev trans-activating proteins, antisense molecules or inhibitors of the viral integrase. These compounds may also be co-administered with other inhibitors of HIV aspartyl protease. Furthermore, it may be found useful to administer compounds of the present invention with any other drug (other anti-viral compounds, antibiotics, pain killer, etc.,).

Combination therapies according to this invention exert a synergistic effect in inhibiting HIV replication because each component agent of the combination acts on a different site of HIV replication. The use of such combinations also advantageously reduces the dosage of a given conventional anti-retroviral agent that would be required for a desired therapeutic or prophylactic effect as compared to when that agent is administered as a monotherapy. These combinations may reduce or eliminate the side effects of conventional single anti-retroviral agent therapies while not interfering with the anti-retroviral activity of those agents. These combinations reduce the potential of resistance to single agent therapies, while minimizing any associated toxicity. These combinations may also increase the efficacy of the conventional agent without increasing the associated toxicity. Combination therapies encompassed by the present invention include, for example, the administration of a compound of this invention with AZT, 3TC, ddI, ddC, d4T or other reverse transcriptase inhibitors.

Alternatively, a combination of compounds described herein with a CYP450 inhibitor may also be co-administered with other HIV protease inhibitors such as Ro 31-8959 (Saquinavir; Roche), L-735,524 (Indinavir; Merck), AG-1343 (Nelfinavir; Agouron), ABT-378/r (Lopinavir; Abbott), and VX-478 (Amprenavir; Glaxo) to increase the effect of therapy or prophylaxis against various viral mutants or members of other HIV quasi species.

Administration of compounds of the present invention may be performed, for example, as single agents or in combination with retroviral reverse transcriptase inhibitors, or other HIV aspartyl protease inhibitors. Co-administration of the compounds of this invention with retroviral reverse transcriptase inhibitors or HIV aspartyl protease inhibitors may exert a substantial synergistic effect, thereby preventing, substantially reducing, or completely eliminating viral infectivity and its associated symptoms.

The compounds of the present invention may be administered in such a manner or form which may allow cleavage of the R₁ unit to release a protease inhibitor. The compounds of this invention may also be administered, for example, in combination with immunomodulators (e.g., bropirimine, anti-human alpha interferon antibody, IL-2, GM-CSF, methionine enkephalin, interferon alpha, diethyldithiocarbamate sodium, tumor necrosis factor, naltrexone and rEPO) antibiotics (e.g., pentamidine isethionate) or vaccines to prevent or combat infection and disease associated with HIV infection, such as AIDS and ARC.

When the combination of compounds described herein with a CYP450 inhibitor are administered in combination therapies with other agents, they may be administered sequentially or concurrently to the patient. Alternatively, pharmaceutical or prophylactic compositions according to this invention may be comprised of a combination of one or more compounds of this invention and another therapeutic or prophylactic agent.

Although this invention focuses on the use of the combination disclosed herein for preventing and treating HIV infection, the combination of this invention may also be used as inhibitory agents for other viruses that depend on similar aspartyl proteases for obligatory events in their life cycle. These viruses include, but are not limited to, retroviruses causing AIDS-like diseases such as simian immunodeficiency viruses, HIV-2, HTLV-I and HTLV-II. In addition, the combination of this invention may also be used to inhibit other aspartyl proteases and, in particular, other human aspartyl proteases including renin and aspartyl proteases that process endothelin precursors.

Pharmaceutical compositions of this invention comprise any of the compounds of the present invention, and pharmaceutically acceptable salts thereof, with any pharmaceutically acceptable carrier, adjuvant or vehicle. Pharmaceutically acceptable carriers, adjuvants and vehicles that may be used in the pharmaceutical compositions of this invention include, but are not limited to ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethyleneglycol, sodium carboxymethylcellulose, polyacrylates, waxes, polyethylene-polyoxypropylene-block polymers, polyethylene glycol and wool fat.

The pharmaceutical compositions of this invention may be administered orally, parenterally by inhalation spray, topically, rectally, nasally, buccally, vaginally or via an implanted reservoir. It is therefore understood herein that oral administration or administration by injection are encompassed by the present invention. For example, compounds of the present invention, may, for example, be orally administered in an aqueous solution. The pharmaceutical compositions of this invention may contain any conventional non-toxic pharmaceutically acceptable carriers, adjuvants or vehicles. The term “parenteral” as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are amino acid, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv. or a similar alcohol.

The pharmaceutical compositions of this invention may be orally administered in any orally acceptable dosage form including, but not limited to, capsules, tablets, and aqueous suspension and solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

The pharmaceutical compositions of this invention may also be administered in the form of suppositories for rectal administration. These compositions may be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene or polyoxypropylene compound, emulsifying wax and water. Alternatively, the pharmaceutical compositions may be formulated with a suitable lotion or cream containing the active compound suspended or dissolved in a carrier. Suitable carriers include, but are not limited to, mineral oil, sorbitan monostearate, polysorbate 60, cetyl esters wax cetearyl alcohol, 2-octyldodecanol, benzyl alcohol and water. The pharmaceutical compositions of this invention may also be topically applied to the lower intestinal tract by rectal suppository formulation or in a suitable neat formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline employing benzyl alcohol or other suitable preservatives, absorption promoters to enhance bioavailability, fluorocarbons, and/or other solubilizing or dispersing agents known in the art.

Dosage levels of between about 0.01 and about 150 mg/kg body weight per day of for example, 0.01 and about 50 mg/kg body weight per day or for example from between about 0.5 and about 30 mg/kg body weight per day of the active ingredient compound are useful in the prevention and treatment of viral infection, including HIV infection. Typically, the pharmaceutical compositions of this invention will be administered from about 1 to about 5 times per day or alternatively, as a continuous infusion. Such administration may be used as a chronic or acute therapy. The amount of active ingredient that may be combined with the carrier materials to produce a single dosage form will vary depending upon the patient treated and the particular mode of administration. A typical preparation will contain from about 5% to about 95% of a desired compound (w/w) i.e., active ingredient or a precursor. For example, such preparations may contain from about 20% to about 80% of a desired compound.

Upon improvement of a patient's condition, a maintenance dose of a compound, composition or combination of this invention may be administered if necessary. Subsequently, the dosage or frequency of administration, or both, may be reduced, as a function of the symptoms, to a level at which the improved condition is retained. When the symptoms have been alleviated to the desired level, treatment should cease. Patients may, however, require intermittent treatment on a long-term basis, upon any recurrence of disease symptoms.

As the person skilled in the art will appreciate, lower or higher doses than those recited above may be desired. Specific dosage and treatment regimen for any particular patient may depend upon a variety of factors, including the activity of the specific compound employed, the age, body weight, general health status, sex, diet, time of administration, rate of excretion, drug combination, the severity and course of the infection, the patient's disposition to the infection and the judgment of the treating physician.

In the description herein, the following abbreviations are used:

Abbreviation Meaning
Ac Acetyl
AcOH Acetic acid
APCl Atmospheric pressure chemical ionization
AIDS Acquired Immunodeficiency Syndrome
APV Amprenavir
ATV Atazanavir
AZT 3-Azido-3-deoxythymine (Zidovudine)
Boc Benzyloxycarbonyl
t-Butyl tert-Butyl
CAM Cerium ammonium molybdate
DCM Dichloromethane
DMAP N,N-dimethylaminopyridine
DMSO Dimethylsulfoxide
DMF Dimethylformamide
DNA Deoxyribonucleic acid
EDAC 1-[3-(dimethylamino)propyl]-3-ethylcarbodiimide hydrochloride
EtOAc Ethyl acetate
EtOH Ethyl alcohol
g Gram
h hour
HIV-1, -2 Human immunodeficiency virus type 1, type 2
HOBt 1-Hydroxybenzotriazole
HPLC High performance liquid chromatography
HTLV-I, -II Human T-cell lymphotropic virus type I, type II
IL-2 Interleukin-2
IDV Indinavir
Kg Kilogram
L Liter
LC-MS Liquid chromatography-mass spectrometry
LPV Lopinavir
M Molar
MeOH Methyl alcohol
mg Milligram
mp Melting point
min Minute
Moc Methoxycarbonyl
mol Mole
mL Milliliter
mmol Millimole
NFV Nelfinavir
nm Nanometer
nM Nanomolar
po Orally
rEPO Recombinant erythropoietin
RTV Ritonavir
SQV Saquinavir
TLC Thin layer chromatography
3TC 2′,3′-Dideoxy-3-thiacytidine
TFA Trifluoroacetic acid
THF Tetrahydrofuran

### BRIEF DESCRIPTION OF THE DRAWINGS

In drawings which illustrates exemplary embodiments of the present invention;

FIG. 1 is a graph illustrating the fold-change (FC) and median fold-change in IC₅₀ vs the reference strain (NL-4.3) observed for various protease inhibitors;

FIG. 2 is a schematic illustrating the advantages of the Lysine-based compounds disclosed herein;

FIG. 3 is a schematic illustrating the cleavage of the physiologically cleavable unit of a Lysine-based compound (e.g., PL-461) generating the active ingredient (e.g., PL-100);

FIG. 4 is a graph illustrating water solubility of PL-461 at various pH;

FIG. 5 is a graph illustrating the plasma concentration of the drug after administration of either a Lysine-based compound (e.g., PL-461) or an active ingredient (e.g., PL-100) in rats;

FIG. 6 is a histogram illustrating the effect of CYP450 inhibitors on the metabolism of various HIV-1 protease inhibitors in human liver microsomes;

FIG. 7 is a histogram illustrating the bioavailability of a Lysine-based compound (e.g., PL-461) or of an active ingredient (e.g., PL-100) in combination with various concentration of a CYP450 inhibitor (e.g., ritonavir) in rats;

FIG. 8 is a graph illustrating the plasma concentration of the drug after administration of a Lysine-based compound (e.g., PL-461) in combination with a CYP450 inhibitor (e.g., ritonavir) in rats;

FIG. 9 is a histogram illustrating the bioavailability of the drug following administration of various Lysine-based compound (e.g., PL-461)/CYP450 inhibitor (CYP450) ratios in rats and;

FIG. 10 is a table illustrating the inhibition of CYP450 sub-families by the active ingredient PL-100.

### EXAMPLES

This section describes the synthesis of lysine based compounds able to release an HIV aspartyl protease inhibitors as described herein. These examples are for the purpose of illustration only and are not to be construed as limiting the scope of the invention in any way. This section presents the detailed synthesis of compounds no. 1 to 10 of this invention.

Exemplary synthesis schemes of the active ingredients have been disclosed in U.S. Pat. No. 6,632,816 to Stranix et al.

Materials and Methods-Preparation of Compounds

Analytical thin layer chromatography (TLC) was carried out with 0.25 mm silica gel E. Merck 60 F₂₅₄ plates and eluted with the indicated solvent systems. Preparative chromatography was performed by flash chromatography, using silica gel 60 (EM Science) with the indicated solvent systems and positive air pressure to allow proper rate of elution. Detection of the compounds was carried out by exposing eluted plates (analytical or preparative) to iodine, UV light and/or treating analytical plates with a 2% solution of p-anisaldehyde in ethanol containing 3% sulfuric acid and 1% acetic acid followed by heating. Alternatively, analytical plates may be treated with a 0.3% ninhydrin solution in ethanol containing 3% acetic acid and/or a CAM solution made of 20 g (NH₄)₆Mo₇O₂₄ and 8.3 g Ce(SO₄)₂ polyhydrate in water (750 mL) containing concentrated sulfuric acid (90 mL).

Preparative HPLC were performed on a Gilson apparatus equipped with a C18 column, a 215 liquid handler module and 25 mL/min capacity head pumps. The HPLC is operated with a Gilson UniPoint System Software.

Semi-Preparative HPLC Conditions for Purification of Test Compounds:

HPLC system: 2 Gilson #305-25 mL pumps, Gilson #215 liquid handler for injection and collection and a Gilson #155 UV-Vis absorbance detector, all controlled from a Gilson Unipoint V1.91 software

Column: Alltech (#96053) Hyperprep PEP, C-18, 100 Åα, 8 μm, 22×250 mm

Flow: 15 mL/min

Solvents: A: H₂O; B: CH₃CN

Gradient: 25% to 80% of B over 40 min

Detector: absorbance; λ: 210 & 265 nm

The crude material dissolved in acetonitrile to a concentration of around 50 to 80 mg/2 mL were injected in each run. Fractions were collected in amounts of 9 mL pertaining absorbance was detected at the UV detector.

Unless otherwise indicated, all starting materials were purchased from a commercial source such as Aldrich Co. or Sigma Co.

Melting points (mp) were determined on a Büchi 530 melting point apparatus in capillary tubes and were uncorrected.

Mass spectra were recorded on a Hewlett Packard LC/MSD 1100 system using APCl or electrospray sources either in negative mode or positive mode.

Nuclear magnetic resonance (NMR) spectra were recorded on a Bruker AMX-II-500 equipped with a reversed or QNP probe. Samples were dissolved in deuterochloroform (CDCl₃), deuteroacetone (acetone-d₆), deuteromethanol (CD₃OD) or deuterodimethylsulfoxide (DMSO-d₆) for data acquisition using tetramethylsilane as internal standard. Chemical shifts 0 are expressed in parts per million (ppm), the coupling constants (J) are expressed in hertz (Hz) whereas multiplicities are denoted as s for singlet, d for doublet, 2d for two doublets, dd for doublet of doublets, t for triplet, q for quartet, quint. for quintet, m for multiplet, and br s for broad singlet.

### DETAILED DESCRIPTION OF THE INVENTION

### Examples

Specific Examples for the Preparation of Derivatives of General Formula I

The preparation of PL-100, PL-337 and other compounds of this class is presented in U.S. Pat. No. 6,632,816 to Stranix et al.

The following compounds were prepared from L-lysine derivatives using the procedures summarized in schemes 1, 1A, 2, 3, 4 and 5 of this invention.

### Example 1

### Preparation of (1S,5S)-(1-{5-[(4-amino-benzenesulfonyl)-isobutyl-amino]-6-phosphonooxy-hexylcarbamoyl}-2,2-diphenyl-ethyl)-carbamic Acid Methyl Ester (PL-461)

The preparation of the title compound is based on schemes 1 and 2 of this invention.

Step A. Preparation of (3S)-3-isobutylamino-azepan-2-one (IV)

L-α-amino-caprolactam (22.0 g) was dissolved in cold dichloroethane (DCM, 200 mL). isobutyraldehyde (12.6 g) was added slowly and stirred until the heat evolved was dissipated (water forms at the surface). The cold solution was added to 46.5 g of powdered NaBH(OAc)₃ in DCM (0.5 L). AcOH (70 mL) was added to the solution. The slightly turbid mixture was stirred at 20° C. for 4 h. A 500 mL solution of 2M NaOH was added slowly to the turbid mixture and the pH adjust to 11 using a concentrated NaOH solution, and then the mixture stirred for a further 20 min. After extraction, the DCM layer was dried with MgSO₄, filtered and evaporated. The oil thus obtained crystallizes slowly on standing (27.8 g, 85%) and was used without further purification in the next step.

¹H NMR (CDCl₃): δ 0.93 (d, J=6.5, 3H), 0.97 (d, J=6.5, 3H), 1.39 (t, J=9.8, 1H), 1.47 (m, 1H), 1.78-1.65 (m, 2H), 2.00-1.93 (m, 2H), 2.32-2.2 (m, 2H), 2.38 (t, J=9.7, 1H), 3.16 (m, 3H), 6.62 (s, 1H(NH)). mp 52-54° C. (hexanes).

A small sample was converted to the S-methyl benzyl urea by adding the solid to a solution of S-methyl benzyl isocyanate in MeCN. NMR gives 98% ee

Step B. Preparation of Nα-isobutyl-Nα-(4-acetamidobenzenesulfonyl)-L-α-amino-caprolactam (V)

Nα-isobutyl-L-α-amino-caprolactam (IV) (4.1 g free base) was dissolved in DCM (200 mL) and treated with 4.0 g triethylamine, followed by 4-acetamidobenzenesulfonyl chloride (5.2 g). A 0.1 g portion of dimethylaminopyridine was added and the mixture was stirred 5 h. The resulting thick slurry was poured into 500 mL 0.5 M HCl and shaken vigorously. The solid in the biphasic solution was filtered out and washed with cold acetone to give 7.3 g (87%) of clean product.

¹H NMR (DMSO-d₆): 0.93 (d, J=6.0, 3H), 0.96 (d, J=6.0, 3H), 1.39 (t, J=12.0, 1H), 1.85-1.65 (m, 3H), 2.08-2.18 (m and s, 6H), 2.90-2.97 (m, 1H), 3.00-3.06 (m, 2H), 3.35 (dd, J=14.2, 8.5, 1H), 4.65 (d, J=8.7, 1H), 6.3 (s, 1H), 7.42 (d, J=8.8, 2H), 7.6 (d, J=8.8, 2H). mp 230-233° C. (EtOH).

Step C. Preparation of (3S)-3-{([4-(acetyl-tert-butoxycarbonyl-amino)-benzenesulfonyl]-isobutyl-amino}-2-oxo-azepane-1-carboxylic acid tert-butyl ester (Boc activation) (VI)

4.2 g of Nα-isobutyl-Nα-(4-acetamidobenzenesulfonyl)-L-α-amino-caprolactam (V) was suspended in 30 mL MeCN and briefly sonicated to break up any large chunks. To this white suspension was added 6.7 g (3 eq.) of di-tert-butyl pyrocarbonate in 10 mL MeCN. The suspension was stirred with a magnetic bar and a 120 mg portion of DMAP was added. The solution becomes a clear light yellow after a few minutes. TLC (EtOAc) reveals 1 product Rf 0.9 (starting material Rf at 0.4). The solution is poured in distilled water 20 mL and extracted with ether, dried with Na₂SO₄ and evaporated yielding 6.90 g. A sample was recrystallized from hexanes.

¹H NMR (DMSO-d₆): 0.68 (d, J=6.0, 3H), 0.85 (d, J=6.0, 3H), 1.39 (s, 10H), 1.47 (s, 9H), 1.85-1.65 (m, 3H), 2.15 (s, 3H), 2.80 (q, J=4, 1H), 3.10-3.36 (m, 2H), 4.01 (d, J=8.0, 1H), 4.85 (d, J=8.7, 1H), 7.32 (d, J=8.8, 2H), 7.87 (d, J=8.8, 2H). mp 123-124° C.

Step D. Preparation of (1S)-4-amino-N-(5-amino-1-hydroxymethyl-pentyl)-N-isobutyl-benzenesulfonamide (VII-deprotected) (Reductive Ring Opening and Deprotection)

A 3.0 g portion of (3S)-3-{[4-(acetyl-tert-butoxycarbonyl-amino)-benzenesulfonyl]-isobutyl-amino}-2-oxo-azepane-1-carboxylic acid tert-butyl ester (VI, step C) is dissolved in 40 mL EtOH followed by 750 mg NaBH₄. Brief heating with a heat gun gives a clear solution. TLC reveals one streaky spot after 20 min (EtOAc). The solution is concentrated to a paste, poured in 40 mL 1N NaOH and extracted with ethyl acetate, the organic phase dried with NaSO₄ and evaporated to give 2.8 g of product intermediate (VII); (1S)-{4-[(5-tert-butoxycarbonylamino-1-hydroxymethyl-pentyl)-isobutyl-sulfamoyl]-phenyl}-carbamic acid tert-butyl ester (VII).

The above product intermediate is dissolved in 5 mL EtOH and 5 mL 12 N HCl is added. Vigorous gas evolution is observed for a few minutes. After 2 h the solution is evaporated and rendered basic with concentrated KOH and extracted with EtOAc yielding 1.75 g of a white powder.

¹H NMR (DMSO-d₆): 0.82 (m, 6H), 0.97-1.12 (m, 2H), 1.15-1.30 (m, 3H), 1.57 (m, 1H), 1.84 (m, 1H), 2.40 (t, J=7.8, 2H), 2.75 (m, 1H), 2.85 (m, 1H), 3.21 (m, 1H), 3.44 (d, J=6.4, 2H), 5.92 (br s, 2H), 6.59 (d, J=8.0, 2H), 7.39 (d, J=8.0, 2H).

Step E. Preparation (2S)-2-methoxycarbonylamino-3,3-diphenyl-propionic acid

To a solution of L-diphenylalanine (241 mg, 1.0 mmol) (Peptech Corp.) in 5 mL 1N NaOH and 0.5 mL saturated Na₂CO₃ (resulting solution at pH 10) was added methoxycarbonyloxysuccinimide (carbonic acid 2,5-dioxo-pyrrolidin-1-yl ester methyl ester) (180 mg, 1.1 mmol) dissolved in 5 mL. Afterwards, the reaction mixture was stirred at room temperature for 2 h. The alkaline solution was extracted once with ether (10 mL) and the aqueous phase was acidified with 1N HCl. This was extracted twice with 20 mL EtOAc, and the combined organic phases were washed with 50 mL 1N HCl. The organic phase was dried over Na₂SO₄ filtered and evaporated to an oil, which solidifies to yields 250 mg (83%) of the desired material. This derivative was used as such in the next step.

Step F. Preparation of (1S,5S)-(1-{5-[(4-amino-benzenesulfonyl)-isobutyl-amino]-6-hydroxy-hexylcarbamoyl}-2,2-diphenyl-ethyl)-carbamic acid methyl ester (PL-100)

The title compound was prepared from (1S)-4-amino-N-(5-amino-1-hydroxymethyl-pentyl)-N-isobutyl-benzenesulfonamide (VII-deprotected) (step D) and (2S)-2-methoxycarbonylamino-3,3-diphenyl-propionic acid (step E) using the coupling procedure with HOBt and EDAC described in example 3 (step D). The final product was obtained in 67% yield (121 mg).

LC-MS: 625.3 (M+H)⁺, 95% pure

¹H NMR (CD₃OD): δ 0.71-0.85 (m, 2H), 0.88 (d, J=6.3, 5H), 0.91-0.96 (m, 2H), 1.29-1.34 (m, 1H), 1.41-1.52 (m, 1H) 1.82-1.92 (m, 1H), 2.61-2.68 (m, 1H), 2.81-2.85 (m, 2H), 2.94-3.05 (m, 2H), 3.38-3.40 (t, J=5.0, 1H), 3.50-3.51 (m, 1H), 3.52 (s, 3H), 4.28 (d, J=11.0 1H), 4.87 (d, J=11.0, 1H), 6.69 (d, J=8.0, 2H), 7.15-718 (m, 2H), 7.20-7.31 (m, 6H), 7.33 (d, J=7.9, 2H), 7.47 (d, J=7.5, 1H).

¹³C NMR (CD₃OD): δ 20.0, 20.1, 23.3, 25.4, 28.1, 28.5, 28.9, 38.1, 40.0, 51.2, 51.6, 53.1, 57.2, 57.4, 59.5, 61.9, 62.4, 112.6, 125.7, 126.2, 126.3, 127.9, 128.1, 128.15, 128.2, 128.4, 128.7, 141.3, 141.9, 152.4, 155.9, 169.9, 172.5.

Step G. Preparation of (1S,5S)-{1-[5-[(4-amino-benzenesulfonyl)-isobutyl-amino]-6-(diethoxy-phosphoryloxy)-hexylcarbamoyl]-2,2-diphenyl-ethyl}-carbamic acid methyl ester

The PL-100 compound (product of step F, 203 mg, 0.325 mmol) was dissolved in dry tetrahydrofuran (3 mL) and 0.2 mL triethylphosphate under N₂ atmosphere. The mixture was stirred at this temperature for 15 min, followed by the addition of diethyl chlorophosphate (0.061 mL, 0.423 mmol). Sodium hydride (60% in mineral oil) (17 mg, 0.423 mmol) was added at 0° C. The solution was stirred for 1 h at 0° C. and 12 h at room temperature. 20 mL of Amberlite XAD-2 was added to the solution and the beads were thoroughly mixed with the solvent. To the mixture was added ice water 2 mL, and the THF evaporated off. The beads were then washed with distilled water 6 times 100 mL then extracted three times with ethyl acetate (30 mL). The combined phase was evaporated and the residue was dried under high vacuum. The crude product was purified by flash chromatography using ethyl acetate/hexane (8/2), then EtOAc 100% as eluent. The yield of this reaction is 152 mg 61%.

LC-MS: 761.2 (M+H)⁺, 90% pure

¹H NMR (CD₃OD): δ 0.68-0.75 (m, 1H), 0.75-0.84 (m, 1H), 0.84-1.10 (m, 9H), 1.21-1.50 (m, 8H), 1.88 (m, 1H), 2.58-2.71 (m, 1H), 2.80-2.89 (m, 1H), 2.89-3.08 (m, 2H), 3.49-3.60 (s, 3H), 3.65-3.74 (m, 1H), 3.85-3.95 (m, 1H), 3.97-4.02 (m, 1H), 4.07-4.21 (m, 4H), 4.29 (d, J=10.8, 1H), 6.71 (d, J=8.0, 2H), 7.10-7.20 (m, 2H), 7.20-7.32 (m, 5H), 7.32-7.45 (m, 3H), 7.50 (d, J=7.5, 2H), 7.86 (br s, 1H).

³¹P NMR (CD₃OD): δ 1.62

Step H. Preparation of (1S,5S)-(1-{5-[(4-amino-benzenesulfonyl)-isobutyl-amino]-6-phosphonooxy-hexylcarbamoyl}-2,2-diphenyl-ethyl)-carbamic acid methyl ester (PL-461)

The product of step G prepared above (152 mg) was dissolved in anhydrous dichloromethane (3.0 mL). Trimethylsilyl bromide (0.5 mL) was added at 0° C. The mixture was stirred during 1 h at this temperature and overnight at room temperature. The solvent was evaporated and 0.2 mL water was added to the residue. 3 mL EtOH was added mixed and evaporated. This step was repeated three times and the residue dried in vacuo. Yields 98 mg 70% of the title derivatives of this first example.

LC-MS: 705.2 (M+H)⁺, 95% pure

¹H NMR (CD₃OD): δ 0.65-0.73 (m, 1H), 0.75-0.83 (m, 1H), 0.89 (d, J=5.6, 8H), 1.27-1.38, (m, 1H), 1.42-4.55 (m, 1H), 1.82-1.94 (m, 1H), 2.57-2.68 (m, 1H), 2.78-2.90 (m, 1H), 2.91-3.09 (m, 2H), 3.54 (s, 3H), 3.60-3.72 (m, 1H), 3.87-4.05 (m, 1H), 4.00 (m, 1H), 4.29 (d, J=11.3, 1H), 4.90 (d, J=11.4, 1H), 6.73 (d, J=8.0, 2H), 7.13-7.22 (m, 2H), 7.22-7.33 (m, 6H), 7.33-7.45 (m, 2H), 7.51 (d, J=7.5, 2H).

³¹P NMR (CD₃OD): δ 2.80

### Example 2

### Preparation of (1S,5S)-(1-{5-[(4-amino-benzenesulfonyl)-isobutyl-amino]-6-phosphonooxy-hexylcarbamoyl}-2,2-diphenyl-ethyl)-carbamic Acid Methyl Ester Sodium Salt (PL-462)

70.7 mg of the final product of example 1 is added to 1 mL 0.1 N NaOH and diluted with 1 mL of distilled water. The Solution is then frozen and lyophilized. Yields 67.2 mg (92%) of the desired material with 95% purity.

¹H NMR (CD₃OD): δ 0.72-0.83 (m, 1H), 0.90 (d, J=5.8, 9H), 1.26-1.38 (m, 1H), 1.53-1.65 (m, 1H), 1.88-2.00 (m, 1H), 2.60-2.70 (m, 1H), 2.79-2.89 (m, 1H), 2.98-3.00 (m, 1H), 3.00-3.08 (m, 1H), 3.54 (s, 3H), 3.58-3.71 (m, 1H), 3.72-3.83 (m, 1H), 3.84-3.95 (m, 1H), 4.28 (d, J=11.1, 1H), 4.91 (d, J=11.0, 1H), 6.70 (d, J=7.6, 2H), 7.12-7.22 (m, 2H), 7.22-7.32 (m, 6H), 7.33-7.40 (m, 2H), 7.50 (d, J=7.7, 2H).

³¹P NMR (CD₃OD): δ 3.13

### Example 3

### Preparation of (1S,5S)-(1-{5-[(4-amino-benzenesulfonyl)-isobutyl-amino]-6-phosphonooxy-hexylcarbamoyl}-2-naphthalen-2-yl-ethyl)-carbamic Acid Methyl Ester (PL-507)

The preparation of the title compound is based on scheme 2 of this invention.

Step A. Preparation of (1S)-(4-{[5-tert-butoxycarbonylamino-1-(diethoxyphosphoryloxymethyl)-pentyl]-isobutyl-sulfamoyl)phenyl)-carbamic acid tert-butyl ester (VII)

2.00 g (3.7 mmol) (1S)-(4-[(5-tert-butoxycarbonylamino-1-hydroxymethyl-pentyl)-isobutyl-sulfamoyl]-phenyl}-carbamic acid tert-butyl ester (VII) (example 1, step D) is dissolved in 0.63 mL triethylphosphate and 10 mL THF at 0° C. under inert argon atmosphere. 0.63 mL (4.44 mmol) diethylchlorophosphate is added and then 0.25 g (6.2 mmol), NaH 60% in oil is added in portionwise. The mixture is allowed to warm to room temperature and left to stir for 2 h (LC-MS showed completion after 1 h). To the solution is added 20 mL of Amberlite XAD-2 resin and the slurry thoroughly mixed and added to 200 mL ice water. After stirring for 15 min. the resin suspension is filtered and the resin washed several times with distilled water (500 mL). The desired product is desorbed from the resin with acetone (5×50 mL), EtOAc (5×50 mL), the organic phase is then dried over Na₂SO₄. After evaporation of the solvent 2.66 g (89%) of clear oil is obtained. The crude product contains a fraction with two diethyl phosphates and is used as is in the next step.

¹H NMR (CD₃OD): δ 0.91 (d, J=6.3, 6H), 1.11-1.21 (m, 2H), 1.33 (t, J=6.9, 10H), 1.43 (s, 9H), 1.53 (s, 10H), 1.90-1.97 (m, 1H), 2.88-2.96 (m, 3H), 2.96-3.04 (m, 1H), 3.81-3.90 (m, 1H), 3.91-3.99 (m, 1H), 4.01-4.14 (m, 4H), 7.61 (d, J=8.3, 2H), 7.72 (d, J=8.4, 2H).

³¹P NMR (CD₃OD): δ 1.59

Step B. Preparation of (2S)-phosphoric acid 6-amino-2-[(4-amino-benzenesulfonyl)-isobutyl-amino]-hexyl ester diethyl ester (IX)

The crude product obtained in the previous step (VIII, 2.66 g) is dissolved in 12 mL EtOH. 4 mL of HCl_{conc}, is added and heated briefly to 70° C. then left at room temperature for 3 h. The solvent is evacuated and the residue triturated with 50 mL ether. The thick residue is then dissolved in 3 mL ice water and the pH adjusted to 12 with 50% NaOH. The thick slurry obtained is extracted with EtOAc (3×50 mL) and the organic phase dried over Na₂SO₄. After filtration of the drying agent the organic phase is evacuated to yield 1.84 g (98%) of the desired product (IX).

LC-MS: 480.2 (M+H)⁺, 95% pure.

¹H NMR (CD₃OD): δ 0.91 (s, 6H), 1.11-1.26 (m, 3H), 1.28-1.43 (m, 8H), 1.45-1.51 (m, 1H), 1.52-1.61 (m, 1H), 1.89-1.96 (m, 1H), 2.56 (t, J=6.7, 2H), 2.85-2.91 (m, 1H), 2.98-3.11 (m, 1H), 3.79-3.99 (m, 1H), 3.94 (d, J=5.3, 1H), 4.09-4.11 (m, 4H), 6.69 (d, J=7.9, 2H), 7.50 (d, J=7.9, 2H).

³¹P NMR (CD₃OD): δ1.61

Step C. Preparation of (2S)-2-methoxycarbonylamino-3-naphthalen-2-yl-propionic acid (or L-Moc-2-naphthylalanine)

To a solution of L-2-naphthylalanine (215 mg, 1 mmol) (Peptech Corp.) in 5 mL 1N NaOH and 0.5 mL saturated Na₂CO₃ (resulting solution at pH 10) was added methoxycarbonyloxysuccinimide (187 mg, 1.1 mmol) dissolved in 5 mL. Afterwards, the reaction mixture was stirred at room temperature for 2 h. The alkaline solution was extracted once with ether (10 mL) and the aqueous phase was acidified with 1N HCl. This was extracted twice with 20 mL EtOAc, and the combined organic phases were washed with 50 mL 1N HCl. The organic phase was dried over Na₂SO₄, filtered and evaporated to an oil, which solidifies to yields 200 mg (73%) of the desired material. This intermediate (referred as the N-substituted amino acid) was used without further purification in the next step.

Step D. Preparation of (1S,5S)-(1-{5-[(4-amino-benzenesulfonyl)-isobutyl-amino]-6-phosphonooxy-hexylcarbamoyl}-2-naphthalen-2-yl-ethyl)-carbamic acid methyl ester (PL-507)

100 mg L-Moc-2-naphthylalanine (step C) was activated with 100 mg EDAC and 57 mg HOBt in 1.5 mL DMF for 30 minutes. Then, 100 mg of phosphoric acid 6-amino-2-[(4-amino-benzenesulfonyl)-isobutyl-amino]-hexyl ester diethyl ester (step B) was added and left to stir at room temperature for 1 h. 40 mL of 1M K₂CO₃ was added to the DMF solution and left for 10 min. 50 mL of EtOAc was then added and the mixture was then agitated vigorously. Separation of the EtOAc phase was effected, followed by extraction with 5% citric acid (50 mL) once, then water (50 mL) 3 times and finally brine. The organic phase was the separated and evaporated. The residue was taken up in 50 mL DCM and re-evaporated. The residue was again taken up in 50 mL DCM and 0.5 mL of TMSBr was added. The solution was left overnight (16 h). The DCM was evacuated and a solution of ice cold MeOH: Water 1:1 was added, stirred briefly and evacuated. The residue was triturated with ether then dissolved in 1N NaOH. The clear solution was extracted with ether and the aqueous phase acidified with 6N HCl. The white precipitated was then collected by filtration and dried in vacuo overnight. Yields 88 mg of the title compound.

LC-MS: 679.8 (M+H)⁺, 95% pure.

¹H NMR (CD₃OD): δ 0.89-0.98 (m, 8H), 1.15 (m, 2H), 1.35 (m, 1H), 1.45 (m, 1H), 1.88 (m, 1H), 2.84 (m, 2H), 2.98 (m, 1H), 3.01 (m, 2H), 3.24 (m, 1H), 3.56 (s, 3H), 3.60 (m, 1H), 3.81 (m, 1H), 3.99 (m, 1H), 4.39 (t, J=6.8, 1H), 6.91 (d, J=8.0, 2H), 7.34 (d, J=8.0, 1H), 7.45 (m, 2H), 7.58 (d, J=8.0, 2H), 7.66 (s, 1H), 7.70-7.82 (m, 3H).

³¹P NMR (CD₃OD): δ 2.56

### Example 4

### Preparation of (2S,2S)phosphoric acid mono-(2-[(4-amino-benzenesulfonyl)-isobutyl-amino]-6-{2-[(morpholine-4-carbonyl)-amino]-3-naphthalen-1-yl-propionylamino}-hexyl)ester (PL-498)

Step A. Preparation of (2S)-2-[(morpholine-4-carbonyl)-amino]-3-naphthalen-1-yl-propionic acid

To a solution of L-1-naphthylalanine (215 mg, 1 mmol) (Peptech Corp.) in 5 mL 1N NaOH and 0.5 mL saturated Na₂CO₃ (resulting solution at pH 10) was added morpholine-4-carbonyl chloride (150 mg, 1.0 mmol) dissolved in 5 mL. Afterwards, the reaction mixture was stirred at room temperature for 2 h. The alkaline solution was extracted once with ether (10 mL) and the aqueous phase was acidified with 1N HCl. This was extracted twice with 20 mL EtOAc, and the combined organic phases were washed with 50 mL 1N HCl. The organic phase was dried over Na₂SO₄ filtered and evaporated to an oil, which solidifies to yields 125 mg (38%) of the desired material. This compound was used as such in the next step.

Step B. Preparation of (2S,2S) Phosphoric acid mono-(2-[(4-amino-benzenesulfonyl)-isobutyl-amino]-6-{2-[(morpholine-4-carbonyl)-amino]-3-naphthalen-1-yl-propionylamino}-hexyl) ester (PL-498)

This compound was made as for the preparation of the product of example 3 (step D) with 100 mg of (2S)-2-[(morpholine-4-carbonyl)-amino]-3-naphthalen-1-yl-propionic acid (step A of this example). The resulting precipitated residue was further purified by reverse phase preparative HPLC. Yields 41 mg of the final compound.

LC-MS: 734.8 (M+H)⁺, 95% pure.

¹H NMR (CD₃OD): δ 0.83-0.98 (m, 8H), 1.00-1.25 (m, 4H), 1.45-1.52 (m, 1H), 1.52-1.66 (m, 1H), 1.88-1.99 (m, 1H), 2.77-2.92 (m, 2H), 2.98-3.16 (m, 3H), 3.40-3.49 (m, 1H), 3.50-3.56 (m, 6H), 3.67-3.69 (m, 1H), 3.81-3.89 (m, 1H), 3.99-4.05 (m, 1H), 4.59 (t, J=6.0, 1H), 6.75 (d, J=8.0, 2H), 7.30-7.60 (m, 7H), 7.75 (d, J=8.0, 1H), 7.90 (d, J=7.8, 1H), 8.23 (d, J=7.8 2H).

³¹P NMR (CD₃OD): δ 2.71

### Example 5

### Preparation of (2S,2S)-phosphoric acid mono-{6-(2-acetylamino-3,3-diphenyl-propionylamino)-2-[(4-amino-benzenesulfonyl)-isobutyl-amino]-hexyl}ester (PL-504)

Step A. Preparation (2S)-2-acetylamino-3,3-diphenyl-propionic acid

To a solution of L-diphenylalanine (100 mg, 0.4 mmol) (Peptech Corp.) in 5 mL 1N NaOH and 0.5 mL saturated Na₂CO₃ (resulting solution at pH 10) was added acetyl chloride (0.5 mmol) dissolved in 5 mL. Afterwards, the reaction mixture was stirred at room temperature for 2 h. The alkaline solution was extracted once with ether (10 mL) and the aqueous phase was acidified with 1N HCl. This was extracted twice with 20 mL EtOAc, and the combined organic phases were washed with 50 mL 1N HCl. The organic phase was dried over Na₂SO₄ filtered and evaporated to an oil, which solidifies to yields 70 mg (60%) of the desired material. This crude intermediate was used as such in the next step.

Step B. Preparation of (2S,2S)-phosphoric acid mono-{6-(2-acetylamino-3,3-diphenyl-propionylamino)-2-[(4-amino-benzenesulfonyl)-isobutyl-amino]-hexyl}ester (PL-504)

This compound was made as for the preparation of the product of example 3 (step D) with 100 mg of (2S)-2-acetylamino-3,3-diphenyl-propionic acid (this example step A). The final product was obtained in 30% yield (30 mg).

LC-MS: 689.3 (M+H)⁺, 95% pure.

¹H NMR (CD₃OD): δ 0.77-1.04 (m, 9H), 1.10-1.17 (m, 1H), 1.23-1.49 (m, 1H), 1.46-1.57 (m, 1H), 1.78 (s, 3H), 1.88-1.99 (m, 1H), 2.80-2.92 (m, 2H), 2.92-3.08 (m, 2H), 3.63-3.75 (m, 1H), 3.79-3.95 (m, 1H), 4.00 (m, 1H), 4.34 (d, J=11.3, 1H), 5.19-5.28 (m, 1H), 6.77-6.85 (m, 2H), 7.10-7.20 (m, 2H), 7.27-7.33 (m, 6H), 7.32-7.41 (m, 2H), 7.49-7.62 (m, 2H).

³¹P NMR (CD₃OD): δ 2.70

### Example 6

### Preparation of (1S,5S)-(1-{5-[(4-amino-3-fluoro-benzenesulfonyl)-isobutyl-amino]-6-phosphonooxy-hexylcarbamoyl)-2,2-diphenyl-ethyl)-carbamic Acid Methyl Ester (PL-515)

First methodology: The preparation of the title compound is based on scheme 3 of this invention.

Step A. Preparation of (1-{5-[(4-amino-3-fluoro-benzenesulfonyl)-isobutyl-amino]-6-hydroxy-hexylcarbamoyl}-2,2-diphenyl-ethyl)-carbamic acid methyl ester (X) (PL-337)

The product of example 1, step F (0.624 g, 1 mmol) is dissolved in 5 mL MeCN at 24° C. SelectFluor 0.35 g (1 mmol) is added in one portion and stirred for 1 h. 1 mL of water is added and the solution was injected directly into a preparative reverse-phase HPLC. The product was collected and lyophilized to give 250 mg (38%) yield of a white solid.

LC-MS: 643.3 (M+H)⁺, 99% pure.

¹H NMR (MeOD): δ 0.71-0.85 (m 2H), 0.88 (d, J=6.3, 6H), 0.91-0.96 (m, 2H), 1.21-1.29 (m, 1H), 1.41-1.52 (m, 1H) 1.82-1.92 (m, 1H), 2.61-2.68 (m, 1H), 2.81-2.85 (m, 2H), 2.94-3.05 (m, 2H), 3.38-3.40 (t, J=5, 1H), 3.49-3.52 (m, 5H), 4.28 (d, J=10, 1H), 4.87 (d, J=10, 1H) 6.90 (t, J=8.3, 1H), 7.20 (m, 2H), 7.28 (m, 3H), 7.33 (m, 3H), 7.39 (m, 4H).

Step B. Preparation of (1S,5S)-(1-[5-[(4-amino-3-fluoro-benzenesulfonyl)-isobutyl-amino]-6-(diethoxy-phosphoryloxy)-hexylcarbamoyl]-2,2-diphenyl-ethyl)carbamic acid methyl ester

The product of step A was phosphorylated with chlorodiethylphosphate following the procedure described in example 1, step G. Yields 157 mg, 68%.

LC-MS: 779.3 (M+H)⁺, 95% pure.

¹H NMR (CD₃OD): δ 0.82 (m, 1H), 0.92 (d, J=6.2, 8H), 0.96 (m, 3H), 1.36 (d, J=3.7, 6H), 1.90 (m, 1H), 2.69 (m, 1H), 2.89 (m, 1H), 2.98 (m, 2H), 3.56 (s, 3H), 3.74 (m, 1H), 3.93 (m, 1H), 4.03 (m, 1H), 4.12 (q, J=7.5 and 14.8, 4H), 4.32 (d, J=11.4, 1H), 4.92 (d, J=11.4, 1H), 6.90 (t, J=8.3, 1H), 7.20 (m, 2H), 7.28 (m, 3H), 7.33 (m, 3H), 7.39 (m, 4H).

³¹P NMR (CD₃OD): δ 1.65

Step C. Preparation of (1S,5S)-(1-{5-[(4-amino-3-fluoro-benzenesulfonyl)-isobutyl-amino]-6-phosphonooxy-hexylcarbamoyl}-2,2-diphenyl-ethyl)-carbamic acid methyl ester (XI) (PL-515)

Deprotection was effected using the procedure described in example 1, step G. Yields 101 mg.

LC-MS: 723.2 (M+H)⁺, 95% pure.

¹H NMR (CD₃OD): δ 0.65-0.77 (m, 1H), 0.77-0.85 (m, 1H), 0.85-1.05 (m, 9H), 1.25-1.39 (m, 1H), 1.40-1.52 (m, 1H), 1.82-1.98 (m, 1H), 2.58-2.72 (m, 1H), 2.82-2.92 (m, 1H), 2.92-3.05 (m, 2H), 3.54 (s, 3H), 3.64-3.75 (m, 1H), 3.80-3.92 (m, 1H), 3.91-4.04 (m, 1H), 4.29 (d, J=11.4, 1H), 7.19 (t, J=6.6, 1H), 7.13-7.21 (m, 2H), 7.22-7.33 (m, 6H), 7.34-7.38 (m, 2H), 7.39-7.48 (m, 2H).

³¹P NMR (CD₃OD): δ 2.74

Second methodology: The preparation of the title compound is based on scheme 4 of this invention.

Step A. Preparation (1S,5S)-(1-(5-[(4-amino-benzenesulfonyl)-isobutyl-amino]-6-phosphonooxy-hexylcarbamoyl}-2,2-diphenyl-ethyl)-carbamic acid methyl ester (PL-461)

(2S)-2-methoxycarbonylamino-3,3-diphenyl-propionic acid ((example 1, step E) 0.9 g, 3 mmol) was activated in DMF (5 mL) with EDAC (1.7 g, 9 mmol) and HOBt (1.2 g, 9 mmol). To the solution was added 1.17 g of (2S)-phosphoric acid 6-amino-2-[(4-amino-benzenesulfonyl)-isobutyl-amino]-hexyl ester diethyl ester (IX) (example 3, step B) and the mixture stirred for 3 h. 20 g of Amberlite XAD-2 resin was then added and the beads were left to soak for 10 min. The resin was transferred into a glass filter and washed thoroughly with distilled water (400 mL) and 200 mL of 1M NaHCO₃. The beads were then washed with 4×50 ml portions of MeOH then EtOAc 200 mL. The organic phase was evaporated. The residue was adsorbed onto silica gel and passed through a short silica gel column (EtOAc) to yield 2.4 g (83%) of white solid after evaporation.

NMR identical as in example 1, step H.

Step B. Preparation (1S,5S)-{1-[5-[(4-amino-3-fluoro-benzenesulfonyl)-isobutyl-amino]-6-(diethoxy-phosphoryloxy)-hexylcarbamoyl]-2,2-diphenyl-ethyl}-carbamic acid methyl ester (XII)

The product of step A above, (1S,5S)-(1-{5-[(4-amino-benzenesulfonyl)-isobutyl-amino]-6-phosphonooxy hexylcarbamoyl}-2,2-diphenyl-ethyl)-carbamic acid methyl ester (0.555 g, 0.73 mmol) was dissolved in 5 mL MeCN. Selectfluor (0.26 g, 0.7 mmol) was added and the mixture stirred for 30 min. The mixture was purified by reverse phase preparative HPLC and lyophilized to yield 278 mg (48% yield) white solid.

¹H NMR identical as previous entry, see first methodology above.

Step C. Preparation (1S,5S)-(1-{5-[(4-amino-3-fluoro-benzenesulfonyl)-isobutyl-amino]-6-phosphonooxy-hexylcarbamoyl}-2,2-diphenyl-ethyl)-carbamic acid methyl ester (XIII, in this specific case is compound XI) (PL-515)

The procedure make this derivative was as described in the deprotection step for the methodology above. Yields 139 mg 70% after reverse phase HPLC.

¹H NMR identical as previous entry, see first methodology above.

### Example 7

### Preparation of (2S,2S)-acetic acid 2-[(4-amino-benzenesulfonyl)-isobutyl-amino]-6-(2-methoxycarbonylamino-3,3-diphenyl-propionylamino)-hexyl Ester (PL-521)

The preparation of the title derivative is based on scheme 5 of this invention.

Step A. Preparation of (2S)-acetic acid 6-tert-butoxycarbonylamino-2-[(4-tert-butoxycarbonylamino-benzenesulfonyl)-isobutyl-amino]-hexyl ester (XIV, R_{1A}═CH₃)

To a stirred solution of (1S)-{4-[(5-tert-butoxycarbonylamino-1-hydroxymethyl-pentyl)-isobutyl-sulfamoyl]-phenyl}carbamic acid tert-butyl ester (intermediate product (VII) of example 1, step D, 97 mg, 0.18 mmol) in anhydrous CH₂Cl₂ (3 mL) was added N,N-dimethylaminopyridine (22 mg, 0.18 mmol) and acetic anhydride (0.014 mL, 0.18 mmol). The mixture was stirred at room temperature for 1 hour. The solvent was evaporated. Ethyl acetate (50 mL) was added and the organic layer was washed with water (30 mL), then dried with Na₂SO₄ and concentrated. The residue was purified by flash chromatography eluting with ethyl acetate. The yield obtained was quantitative (100 mg).

LC-MS: 586.2 (M+H)⁺, 95% pure

Step B. Preparation of (2S)-acetic acid 6-amino-2-[(4-amino-benzenesulfonyl)-isobutyl-amino]-hexyl ester (XV, R_{1A}═CH₃)

This derivative was prepared from (2S)-acetic acid 6-tert-butoxycarbonylamino-2-[(4-tert-butoxycarbonylamino-benzenesulfonyl)-isobutyl-amino]-hexyl ester as described in example 15, step B. The yellow solid (66 mg) was used for the next reaction without purification.

LC-MS: 386.2 (M+H)⁺, 95% pure

Step C. Preparation of (2S,2S)-acetic acid 2-[(4-amino-benzenesulfonyl-)isobutyl-amino]-6-(2-methoxycarbonylamino-3,3-diphenyl-propionylamino)-hexyl ester (XVI, R_{1A}═CH₃) (PL-521)

This derivative was prepared from (2S)-acetic acid 6-amino-2-[(4-amino-benzenesulfonyl)-isobutyl-amino]-hexyl ester (product of step B) as described in example 15, step B. The final product was purified by flash chromatography with a mixture of eluents hexane/ethyl acetate (2/8). A yellow solid was obtained in 70% yield (70 mg).

LC-MS: 667.3 (M+H)⁺, 95% pure

¹H NMR(acetone-d₆): δ 0.85-0.97 (m, 12H), 1.21-1.41 (m, 2H), 1.88-2.00 (s, 3H), 2.59-2.69 (m, 1H), 2.83-2.90 (m, 1H), 2.90-3.01 (m, 1H), 3.01-3.10 (br s, 1H), 3.45-3.60 (s, 3H), 3.70-3.80 (m, 1H), 3.93-4.00 (m, 1H), 4.00-4.11 (m, 1H), 4.38-4.45 (d, J=11.0, 1H), 4.89-4.98 (t, J=10.0, 1H), 5.43-5.58 (br s, 1H), 6.28-6.48 (d, J=8.9, 11-1), 6.72-6.83 (d, J=8.0, 2H), 6.85-6.93 (br s, 1H), 7.12-7.22 (t, J=7.4, 1H), 7.21-7.31 (d, J=7.0, 4H), 7.31-7.45 (m, 5H), 7.48-7.57 (d, J=8.0, 2H).

### Example 8

### Preparation of (2S,2S)-nicotinic acid 2-[(4-amino-benzenesulfonyl)-isobutyl-amino]-6-(2-methoxycarbonylamino-3,3-diphenyl-propionylamino)-hexyl Ester (PL-520)

Step A. Preparation of (2S)-nicotinic acid 6-tert-butoxycarbonylamino-2-[(4-tert-butoxycarbonylamino-benzenesulfonyl)-isobutyl-amino]-hexyl ester (XIV, R_{1A}=3-pyridyl)

(1S)-{4-[(5-tert-butoxycarbonylamino-1-hydroxymethyl-pentyl)-isobutyl-sulfamoyl]-phenyl}-carbamic acid tert-butyl ester (intermediate product (VII) of example 1, step D, 130 mg, 0.24 mmol) was dissolved in anhydrous DMF (1 mL) and treated with 0.066 mL (0.48 mmol) of triethylamine followed by EDC (120 mg, 0.65 mmol), HOBt (88 mg, 0.65 mmol) and nicotinic acid (27 mg, 0.22 mmol). The mixture was stirred overnight at room temperature. The product was extracted with ethyl acetate (40 mL) and water (40 mL). The organic phase was separated and dried with Na₂SO₄, then evaporated to give 200 mg of crude product. This compound was purified by flash chromatography with ethyl acetate as the eluent. A clear oil was obtained in 100% yield (150 mg).

LC-MS: 649.3 (M+H)⁺, 95% pure

¹H NMR (acetone-d₆): δ 0.90-1.14 (d, J=5.9, 6H), 1.31-1.42 (m, 2H), 1.48 (s, 9H), 1.51-1.55 (m, 2H), 1.59 (s, 9H), 1.62-1.69 (m, 1H), 1.72-1.83 (m, 1H), 3.00-3.11 (m, 2H), 3.11-3.17 (m, 1H), 3.19-3.27 (m, 1H), 4.15-4.24 (m, 1H), 4.35-4.44 (t, J=9.1, 1H), 4.50-4.58 (dd, J=4.4 and 11.5, 1H), 5.89-5.99 (br s, 1H), 7.53-7.60 (m, 1H), 7.70-7.77 (d, J=8.2, 2H), 7.80-7.87 (d, J=8.2, 2H), 8.24-8.31 (d, J=7.3, 1H), 8.75-8.82 (m, 1H), 8.82-8.88 (m, 1H), 9.12-9.18 (br s, 1H).

Step B. Preparation of (2S)-nicotinic acid 6-amino-2-[(4-amino-benzenesulfonyl)-isobutyl-amino]-hexyl ester (XV, R_{1A}=3-pyridyl)

The product of step A, (2S)-nicotinic acid 6-tert-butoxycarbonylamino-2-[(4-tert-butoxycarbonylamino-benzenesulfonyl)-isobutyl-amino]-hexyl ester (150 mg, 0.23 mmol), was dissolved in CH₂Cl₂ (5 mL) and trifluoroacetic acid (1 mL) was added. The mixture was stirred during 2 hours at room temperature. The solvent was evaporated and the residue was extracted with ethyl acetate (40 mL) and NaOH 1M (40 mL) (pH=10). The organic portion was separated, dried with Na₂SO₄ and evaporated. The residue (100 mg) was used for the next reaction without further purification. The yield was quantitative.

LC-MS: 449.2 (M+H)⁺, 95% pure

Step C. Preparation of (2S,2S)-nicotinic acid 2-[(4-amino-benzenesulfonyl)-isobutyl-amino]-6-(2-methoxycarbonylamino-3,3-diphenyl-propionylamino)-hexyl ester (PL-520)

The product of step B, (2S)-nicotinic acid 6-amino-2-[(4-amino-benzenesulfonyl)-isobutyl-amino]-hexyl ester (100 mg, 0.22 mmol) was dissolved in anhydrous DMF (2 mL) and treated with 0.062 mL (0.45 mmol) of triethylamine followed by EDC (100 mg, 0.56 mmol), HOBt (75 mg, 0.56 mmol) and (2S)-2-methoxycarbonylamino-3,3-diphenyl-propionic acid (56 mg, 0.19 mmol). The mixture was stirred overnight at room temperature. The product was extracted with ethyl acetate (40 mL) and water (40 mL). The organic layer was separated and dried with Na₂SO₄, then evaporated to give 160 mg of crude oil. The residue was purified by flash chromatography with a mixture of eluents hexane/ethyl acetate (2/8). The title compound was obtained as a clear oil in 20% yield (25 mg).

LC-MS: 730.2 (M+H)⁺, 95% pure

¹H NMR (acetone-d₆): δ 0.80-0.97 (m, 9H), 0.97-1.13 (m, 2H), 1.26-1.40 (m, 1H), 1.40-1.57 (m, 1H), 2.61-2.73 (m, 1H), 2.86-2.98 (m, 2H), 3.00-3.17 (m, 2H), 3.45-3.59 (s, 3H), 3.91-4.00 (m, 1H), 4.24-4.34 (m, 1H), 4.34-4.47 (m, 2H), 4.90-4.99 (t, J=9.7, 1H), 6.35-6.44 (m, 1H), 6.68-6.79 (d, J=7.9, 1H), 6.91-7.00 (br s, 1H), 7.13-7.22 (m, 2H), 7.22-7.31 (m, 3H), 7.35-7.48 (m, 4H), 7.49-7.64 (m, 2H), 7.75-7.84 (m, 1H), 8.25-8.36 (m, 1H), 8.76-8.88 (br s, 1H), 9.12-9.26 (br s, 1H).

### Example 9

### Preparation of (2S,2S)-dimethylamino-acetic acid 2-[(4-amino-benzenesulfonyl)-isobutyl-amino]-6-(2-methoxycarbonylamino-3,3-diphenyl-propionylamino)-hexyl Ester (PL-534)

Step A. Preparation of (2S)-dimethylamino-acetic acid 6-tert-butoxycarbonylamino-2-[(4-tert-butoxycarbonylamino-benzenesulfonyl)-isobutyl-amino]-hexyl ester (XIV, R_{1A}=(CH₃)₂NCH₂—)

This title compound was obtained from (1S)-{4-[(5-tert-butoxycarbonylamino-1-hydroxymethyl-pentyl)-isobutyl-sulfamoyl]-phenyl}-carbamic acid tert-butyl ester (intermediate product (VII) of example 1, step D) as described example 15, step A using N,N-dimethylglycine. The clear oil was obtained in 100% yield (150 mg).

LC-MS: 629.3 (M+H)⁺, 95% pure

¹H NMR (acetone-d₆): δ 0.81-0.95 (d, J=6.1, 6H), 1.18-1.30 (m, 2H), 1.32-1.43 (s, 9H), 1.43-1.52 (s, 8H), 1.52-1.62 (m, 1H), 1.93-2.00 (m, 1H), 2.19-2.29 (s, 4H), 2.69-2.80 (m, 4H), 2.90-3.05 (m, 6H), 3.60-3.65 (m, 1H), 3.85-3.97 (m, 1H), 3.98-4.08 (m, 1H), 4.08-4.14 (m, 1H), 5.78-5.88 (m, 1H), 7.68-7.80 (m, 3H), 8.80-8.88 (br s, 1H).

Step B. Preparation of (2S)-dimethylamino-acetic acid 6-amino-2-[(4-amino-benzenesulfonyl)-isobutyl-amino]-hexyl ester (XV, R_{1A}═(CH₃)₂NCH₂—)

The title derivative was prepared from (2S)-dimethylamino-acetic acid 6-tert-butoxycarbonylamino-2-[(4-tert-butoxycarbonylamino-benzenesulfonyl)-isobutyl-amino]-hexyl ester as described in example 15, step B. The final product (100 mg) was used as such in the next step.

LC-MS: 429.3 (M+H)⁺, 90% pure

Step C. Preparation of (2S,2S)-dimethylamino-acetic acid 2-[(4-amino-benzenesulfonyl)-isobutyl-amino]-6-(2-methoxycarbonylamino-3,3-diphenyl-propionylamino)-hexyl ester (PL-534)

This title compound was prepared from (2S)-dimethylamino-acetic acid 6-amino-2-[(4-amino-benzenesulfonyl)-isobutyl-amino]-hexyl ester as described in example 15, step C. The crude product was purified by LC-preparative. The final compound was obtained in 10% yield (10 mg).

LC-MS: 710.3 (M+H)⁺, 92% pure

¹H NMR (acetone-d₆): δ 0.81-0.98 (m, 12H), 1.14-1.30 (m, 2H), 1.31-1.45 (m, 1H), 2.58-2.77 (m, 2H), 2.79-2.90 (m, 2H), 3.42-3.56 (s, 3H), 3.75-3.85 (m, 1H), 3.99-4.17 (m, 3H), 4.23-4.35 (m, 1H), 4.36-4.45 (m, 1H), 4.86-4.96 (m, 1H), 6.33-6.42 (m, 1H), 6.74-6.83 (m, 1H), 6.85-6.90 (m, 1H), 7.12-7.22 (m, 3H), 7.23-7.31 (m, 4H), 7.31-7.44 (m, 5H), 7.47-7.55 (m, 1H), 7.73-7.80 (m, 1H).

### Example 10

### Preparation of (2S,2S)-2-amino-3-methyl-butyric acid 2-[(4-amino-benzenesulfonyl)-isobutyl-amino]-6-(2-methoxycarbonylamino-3,3-diphenyl-propionylamino)-hexyl Ester (PL-530)

Step A. Preparation of (2S)-2-benzyloxycarbonylamino-3-methyl-butyric acid 6-tert-butoxycarbonylamino-2-[(4-tert-butoxycarbonylamino-benzenesulfonyl)-isobutyl-amino]-hexyl ester (XIV, R_{1A}═(CH₃)₂CHCH(NH₂)—)

This title compound was obtained from (1S)-{4-[(5-tert-butoxycarbonylamino-1-hydroxymethyl-pentyl)-isobutyl-sulfamoyl]-phenyl}-carbamic acid tert-butyl ester (intermediate product (VII) of example 1, step D) as described in example 15, step A using (2S)-2-benzyloxycarbonylamino-3-methyl-butyric acid. The crude product was purified by flash chromatography eluting with a mixture of hexane/ethyl acetate (1/1). The yield obtained was 100% (150 mg).

LC-MS: 777.3 (M+H)⁺, 95% pure

¹H NMR (acetone-d₆): δ 0.80-1.00 (m, 14), 1.13-1.28 (s, 2H), 1.30-1.44 (s, 11H), 1.45-1.56 (s, 10), 1.58-1.67 (m, 1H), 2.87-3.04 (m, 4H), 3.84-3.97 (m, 1H), 3.97-4.12 (m, 2H), 4.12-4.21 (m, 1H), 4.99-5.14 (m, 2H), 5.78-5.89 (m, 1H), 6.38-6.52 (m, 1H), 7.24-7.34 (m, 1H), 7.34-7.41 (m, 2H), 7.65-7.83 (m, 4H), 8.77-8.86 (m, 1H).

Step B. Preparation of (2S)-benzyloxycarbonylamino-3-methyl-butyric acid 6-amino-2-[(4-amino-benzenesulfonyl)-isobutyl-amino]-hexyl ester (XV, R_{1A}═(CH₃)₂CHCH(NH₂)—)

This derivative was prepared from (2S)-2-benzyloxycarbonylamino-3-methyl-butyric acid 6-tert-butoxycarbonylamino-2-[(4-tert-butoxycarbonylamino-benzenesulfonyl)-isobutyl-amino]-hexyl ester (product of step A) as described in example 15, step B. The final compound was obtained in quantitative yield (110 mg) and used for the next step without purification.

LC-MS: 577.3 (M+H)⁺, 90% pure

Step C. Preparation of (2S,2S)-2-benzyloxycarbonylamino-3-methyl-butyric acid 2-[(4-amino-benzenesulfonyl)-isobutyl-amino]-6-(2-methoxycarbonylamino-3,3-diphenyl-propionylamino)-hexyl ester

The title compound was obtained from (2S)-benzyloxycarbonylamino-3-methyl-butyric acid 6-amino-2-[(4-amino-benzenesulfonyl)-isobutyl-amino]-hexyl ester (product of step B) as described in example 15, step C. The clear oil was obtained in 86% yield (120 mg).

LC-MS: 858.3 (M+H)⁺, 95% pure

Step D. Preparation of (2S,2S)-2-amino-3-methyl-butyric acid 2-[(4-amino-benzenesulfonyl)-isobutyl-amino]-6-(2-methoxycarbonylamino-3,3-diphenyl-propionylamino)-hexyl ester (PL-530)

To a stirred solution of (2S,2S)-2-benzyloxycarbonylamino-3-methyl-butyric acid 2-[(4-amino-benzenesulfonyl)-isobutyl-amino]-6-(2-methoxycarbonylamino-3,3-diphenyl-propionylamino)-hexyl ester (step C, 120 mg, 0.14 mmol) in anhydrous THF (8 mL), under nitrogen atmosphere, was added palladium 10% wt. on activated carbon (160 mg). The mixture was reacted under hydrogen atmosphere overnight, at room temperature. The solution was filtered and the palladium on carbon was washed with THF (50 mL). The solvent was evaporated and the residue (110 mg) was purified by flash chromatography using ethyl acetate as the eluent. The clear oil was obtained in 47% yield (47 mg).

LC-MS: 796.4 (M+H)⁺, 95% pure

¹H NMR (acetone-d₆): δ 0.84-0.97 (m, 12H), 0.97-1.08 (m, 2H), 1.27-1.43 (m, 3H), 1.49-1.62 (m, 4H), 1.80-1.93 (m, 1H), 1.94-2.00 (m, 1H), 2.36-2.46 (m, 1H), 2.58-2.74 (m, 2H), 2.86-2.96 (m, 3H), 2.99-3.10 (m, 2H), 3.46-3.52 (s, 3H), 3.52-3.60 (m, 2H), 3.75-3.87 (m, 2H), 3.95-4.04 (m, 1H), 4.10-4.18 (m, 1H), 4.37-4.44 (m, 1H), 4.89-4.97 (m, 1H), 5.40-5.48 (m, 1H), 6.30-6.40 (m, 1H), 6.76-6.83 (d, J=8.2, 1H), 6.87-7.03 (m, 2H), 7.14-7.22 (m, 1H), 7.23-7.34 (m, 3H), 7.35-7.45 (m, 4H), 7.50-7.56 (m, 1H), 7.57-7.65 (m, 1H).

### Example 11

### Inhibitory Activity and Cytotoxicity

The active ingredients which are released from the Lysine-based compounds described herein have been shown to inhibit HIV aspartyl protease (U.S. Pat. No. 6,632,816). After oral dosing, plasma levels of the Lysine-based compound; PL-461 in rats are insignificant or under detection limits, compared to plasma levels of the active ingredient; PL-100, indicating that the phosphate moiety of the Lysine-based compound (e.g., PL-461) is metabolically cleaved and majority of this compound is converted to the corresponding active ingredient (e.g., PL-100) in vivo.

The cross-resistance profile of a representative active ingredient; PL-100 against 63 HIV isolates with reduced susceptibility to all approved protease inhibitors (PIs) including atazanavir is tested herein.

Briefly, the antiviral activity of PL-100 was determined using MT4 cells infected with a laboratory adapted HIV strain (NL4-3). For comparison, seven approved protease inhibitors (PIs) were tested in parallel. Result of this experiment is presented in Table 3.

These data suggest that the antiviral activity of PL-100 is comparable to that of the other PIs.

Cytotoxicity of PL-100 and other P is was evaluated in the same cell culture system. The selectivity index (SI) is the ratio of cytotoxicity (CC₅₀) to antiviral activity (EC₅₀). The selective index is similar to the therapeutic index but relates to in vitro studies.

This result indicates that PL-100 is a potent, selective and non-cytotoxic PI.

TABLE 3

| Protease | | | SI |
|---|---|---|---|
| Inhibitor | EC₅₀ (nM) | CC₅₀ (nM) | (CC₅₀/EC₅₀) |
| Atazanavir | 4 | 55,000 | 13,750 |
| Amprenavir | 47 | >100,000 | >2,128 |
| Indinavir | 67 | >100,000 | >1,493 |
| Lopinavir | 19 | 28,000 | 1,474 |
| Nelfinavir | 29 | 8,000 | 276 |
| Ritonavir | 61 | 25,000 | 410 |
| Saquinavir | 12 | 19,000 | 1,583 |
| PL-100 | 16 | 33,000 | 2,063 |

Atazanavir (ATV) is manufactured by BMS (Bristol Myers Squibb), Amprenavir (APV) is manufactured by GSK (Glaxo SmithKline), Indinavir (IDV) is manufactured by Merck, Lopinavir (LPV) is manufactured by Abott, Nelfinavir (NFV) is manufactured by Pfizer, Ritonavir (RTV) is manufactured by Abott, Saquinavir (SQV) is manufactured by Roche.

HIV PI class inhibitors may sometimes demonstrate binding to plasma protein which may affect their potency in vivo. The effect of 40% human serum on the antiviral activity of PL-100 in MT4 cells infected with NL4-3 was therefore evaluated. As shown in Table 4, the addition of human serum reduced the antiviral activity of PL-100 by 6-fold, which result in an increase of its EC₅₀ to 100 nM. This is similar to what is reported for the approved drugs nelfinavir (NFV) and indinavir (IDV). So protein binding of PL-100 is not expected to be a significant issue in the clinic.

TABLE 4

| | EC₅₀ nM (Fold |
|---|---|
| Culture condition | Change) |
| 10% FBS* | 18 (1.0) |
| 10% FBS* + 40% human | 106 (5.9) |
| serum | |

*Fetal bovine serum

### Example 12

### Cross-Resistance Profile

The cross-resistance profile of PL-100 against 63 HIV isolates with reduced susceptibility to all approved protease inhibitors (P is) including atazanavir was thus tested.

The panel of 63 viral strains was selected based on the following rationale: 1) High-level loss of susceptibility to specific P is; 2) High-level loss of susceptibility to multiple P is; 3) Good representation of the primary mutations. This panel consists of resistant viruses from highly PI-experienced patients with high-level PI resistance.

The genotype of these viruses encompasses a wide variety of mutational patterns. We particularly looked for the following primary PI mutations: D30N, L33F/I, M461/L, G48V, I50L/V, V82A/F/S/T, I84V and L90M as defined by the International AIDS Society (IAS)-USA. Table 5 illustrates the repartition and the strength of the chosen panel.

TABLE 5

| No. of | |
|---|---|
| primary PI | No. of viral |
| mutations | strains |
| 0 | 3 |
| 1 | 6 |
| 2 | 11 |
| 3 | 22 |
| 4 | 17 |
| 5 | 3 |
| 6 | 1 |

The mutation mentioned above refers to original (wild type) amino acid (one letter code)/position in the HIV-1 protease/resulting amino acid change(s) (one letter code). The one letter code amino acid is known in the art.

For example: D30N relates to: aspartic acid (D) found at position 30 of HIV-1 protease which has been changed (mutated) for an asparagine (N):

L33F/I relates to: leucine (L) found at position 33 of HIV-1 protease which has been changed (mutated) for either phenylalanine (F) or isoleucine (I); etc.,

Table 6, illustrates the result of the cross-resistance profile of 63 diverse resistant strains.

TABLE 6

| | | | Fold change in IC50 vs. Reference | | | | | | |
|---|---|---|---|---|---|---|---|---|---|
| SAMPLE ID | Key Mutations¹ | Panel ID | ATV | AMP | IDV | LPV | NFV | SQV | PL-100 |
| O3-129572 | 24I/33F/54V/82A/84V | 1 | | 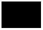 | 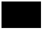 | 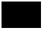 | 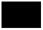 | 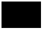 | 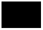 |
| O3-129573 | 24I/33F/46I/54L/82A | 3 | | 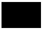 | 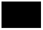 | 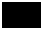 | 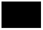 | 1.5 | 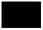 |
| O3-129574 | 54V/82A/84V/90M | 5 | | 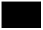 | 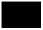 | 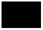 | 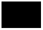 | 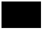 | 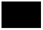 |
| O3-129575 | 33F/54V/73S/82A/84V/90M | 6 | | 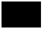 | 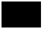 | 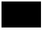 | 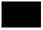 | 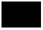 | 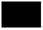 |
| O3-129576 | 33F/46L/54V/82A/84V/90M | 7 | | 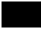 | 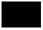 | 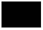 | 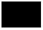 | 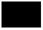 | 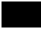 |
| O3-129578 | 46I/82T/84V | 11 | | 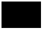 | 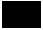 | 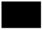 | 1.6 | 1 | 1.2 |
| O3-129579 | 46I/82T/84V | 13 | | 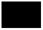 | 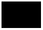 | 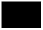 | 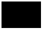 | 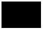 | 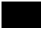 |
| O3-129580 | 46I/82T/84V/90M | 15 | | 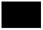 | 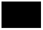 | 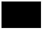 | 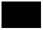 | 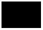 | 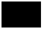 |
| O3-129581 | 33I/46I/84V/88D/90M | 16 | | 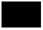 | 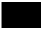 | 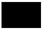 | 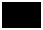 | 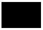 | 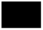 |
| O3-1129583 | 84V/90M | 21 | | 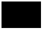 | 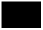 | 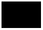 | 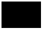 | 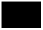 | 2.3 |
| O3-129584 | 33F/50V | 24 | | 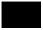 | 1.3 | 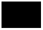 | 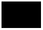 | 1.7 | 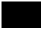 |
| O3-129585 | 48V/54V/82A/90M | 26 | | 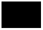 | 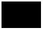 | 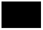 | 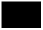 | 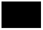 | 1.9 |
| O3-129586 | 30N/88D/90M | 28 | | 1.8 | 2.3 | 1.2 | 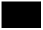 | 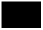 | 1.4 |
| O3-148709² | 32I/47A | 1 | 1 | 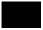 | 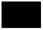 | 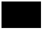 | 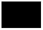 | 0.2 | 1.4 |
| O4-148710² | 46I/47V/84V | 118 | 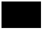 | 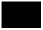 | 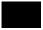 | 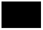 | 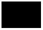 | 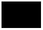 | 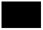 |
| O4-148711 | 46I/47A/84V | 119 | 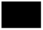 | 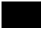 | 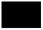 | 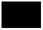 | 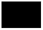 | 1.1 | 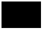 |
| O4-148712 | 46I/88S | 122 | 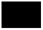 | 0.3 | 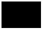 | 1.7 | 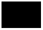 | 1.9 | 0.8 |
| O4-148713 | 46I/88S | 123 | 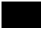 | 0.2 | 2 | 0.7 | 4.6 | 0.6 | 0.4 |
| O4-148712 | 46I/88S | 124 | 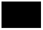 | 0.9 | 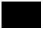 | 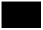 | 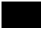 | 2 | 1.1 |
| O4-148715 | 33F/54L/88S/90M | 9 | 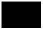 | 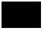 | 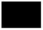 | 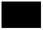 | 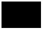 | 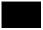 | 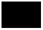 |
| O4-148716 | 33F/54L/88S/90M | 10 | 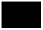 | 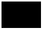 | 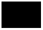 | 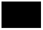 | 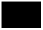 | 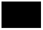 | 1.7 |
| O4-148718 | 33I/46I/84V/88D/90M | 19 | 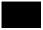 | 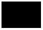 | 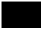 | 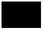 | 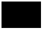 | 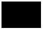 | 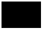 |
| O4-148719 | 33F/46I/84V/88D/90M | 20 | 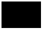 | 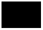 | 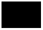 | 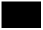 | 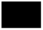 | 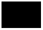 | 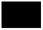 |
| O4-148720 | 30N/33F/46L/54L/84V/88D | 125 | 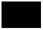 | 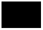 | 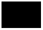 | 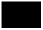 | 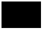 | 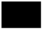 | 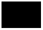 |
| O4-148721 | 32I/46I/47V/50L | 127 | 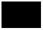 | 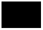 | 2.1 | 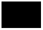 | 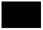 | 0.5 | 1.1 |
| O4-148722 | 33F/54L/82A/90M | 40 | 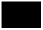 | 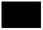 | 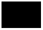 | 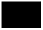 | 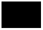 | 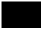 | 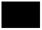 |
| O4-148723 | 33F/54L/82A/90M | 42 | 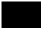 | 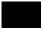 | 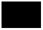 | 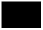 | 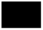 | 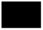 | 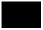 |
| O4-148724 | 33F/54V/73S/82A/90M | 44 | 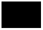 | 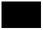 | 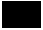 | 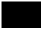 | 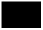 | 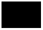 | 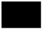 |
| O4-148725 | 32I/46L/47V/84V | 53 | 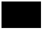 | 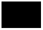 | 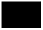 | 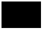 | 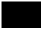 | 0.8 | 0.6 |
| O4-148726 | 33F/54L/82A/84V | 132 | 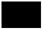 | 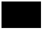 | 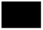 | 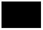 | 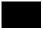 | 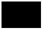 | 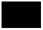 |
| O4-148727 | 33F/54V/82A/84V | 133 | 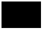 | 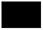 | 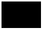 | 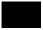 | 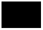 | 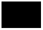 | 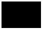 |
| O4-148728 | 33F/46I/53L/82A/84V | 134 | 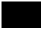 | 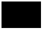 | 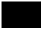 | 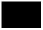 | 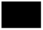 | 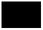 | 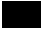 |
| O4-148729 | 33F/54V/82T/84V/90M | 61 | 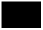 | 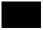 | 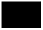 | 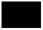 | 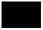 | 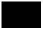 | 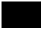 |
| O4-148730 | 33F/46L/53L/54V/82A | 67 | 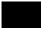 | 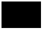 | 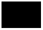 | 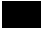 | 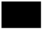 | 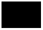 | 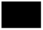 |
| O4-148731 | 46L/54M/82L/84V/90M | 69 | 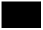 | 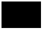 | 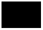 | 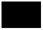 | 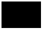 | 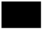 | 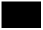 |
| O4-148732 | 46L/54V/82A/90M | 74 | 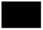 | 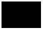 | 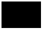 | 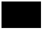 | 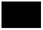 | 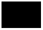 | 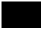 |
| O4-148733 | 46I/54V/82A/84V | 76 | 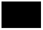 | 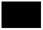 | 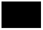 | 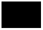 | 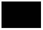 | 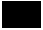 | 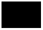 |
| O4-148734 | 46L/54V/73C/84V/90M | 78 | 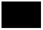 | 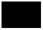 | 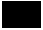 | 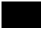 | 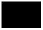 | 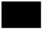 | 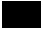 |
| O4-148735 | 46I/82T/84V/90M | 81 | 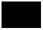 | 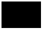 | 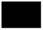 | 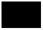 | 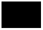 | 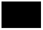 | 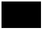 |
| O4-148736 | 46I/82A/84V/90M | 82 | 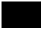 | 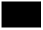 | 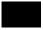 | 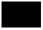 | 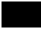 | 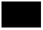 | 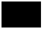 |
| O4-148737 | 54V/82A/84V/90M | 84 | 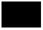 | 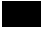 | 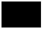 | 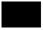 | 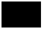 | 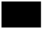 | 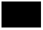 |
| O4-148738 | 33F/54V/82A/84V/90M | 86 | 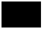 | 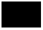 | 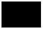 | 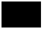 | 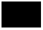 | 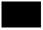 | 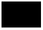 |
| O4-148739 | 48V/54V/82A/90M | 138 | 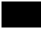 | 1.1 | 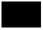 | 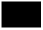 | 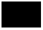 | 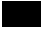 | 1.1 |
| O4-148740 | 46L/48V/82A/84V/90M | 141 | 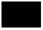 | 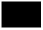 | 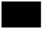 | 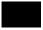 | 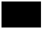 | 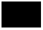 | 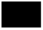 |
| O4-148741 | 48V/82A/84V/90M | 142 | 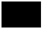 | 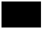 | 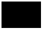 | 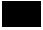 | 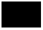 | 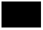 | 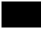 |
| O4-148742 | 46L/48V/82A/84V | 94 | 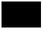 | 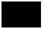 | 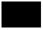 | 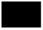 | 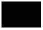 | 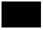 | 1.2 |
| O4-148743 | 46L/48V/82A/90M | 95 | 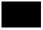 | 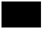 | 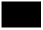 | 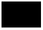 | 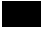 | 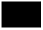 | 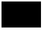 |
| O4-148744 | 46L/50L/54V/82A | 98 | 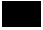 | 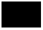 | 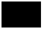 | 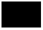 | 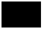 | 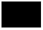 | 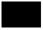 |
| O4-148745 | 46I/50L/54V/82A | 99 | 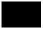 | 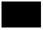 | 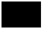 | 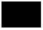 | 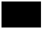 | 2.2 | 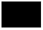 |
| O4-148746 | 33I/46I/84V/90M | 105 | 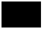 | 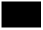 | 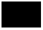 | 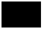 | 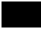 | 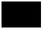 | 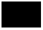 |
| O4-148747 | 33I/46I/84V/90M | 107 | 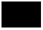 | 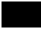 | 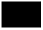 | 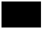 | 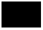 | 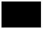 | 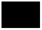 |
| O4-148748 | 37S/41K/70E | 115 | 1.4 | 0.7 | 1.2 | 1 | 1.6 | 1.2 | 0.9 |
| O4-148749 | 37T/41K/70E | 151 | 1.5 | 1.9 | 1.4 | 1.3 | 1.9 | 1.2 | 1.9 |
| O4-148750 | 46I/53L/82T/90M | 172 | 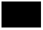 | 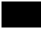 | 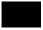 | 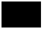 | 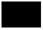 | 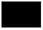 | 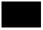 |
| O4-148751 | 54V/84V | 171 | 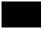 | 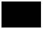 | 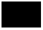 | 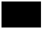 | 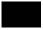 | 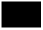 | 0.8 |
| O4-148752 | 54V/84V | 152 | 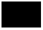 | 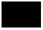 | 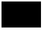 | 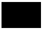 | 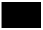 | 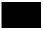 | 1.2 |

In Table 6 above, (¹) indicates that mutations used in selecting viruses in at least one group; mixtures were ignored for this purpose, (²) indicates non-B subtype and (*) indicates 50% inhibition not reached at highest drug concentration tested and that an arbitrary FC value (400) was therefore assigned.

The key mutations listed in Table 6, only refers to the position in the HIV-1 protease which has been mutated and the resulting amino acid (one letter code) found in the mutated protease of the resistant strain. The original amino acid is not indicated.

For example, 33F/46L/54V/82A/84V/90M (panel ID 7 of Table 6) indicates that amino acid at position 33 has been mutated to phenylalanine (F), amino acid at position 46 has been mutated to leucine (L), amino acid at position 54 has been mutated to valine (V), amino acid at position 82 has been mutated to alanine (A), amino acid at position 84 has been mutated to valine (V) and amino acid at position 90 has been mutated to methionine (M). Similarly with the other resistant viral strains listed in Table 6.

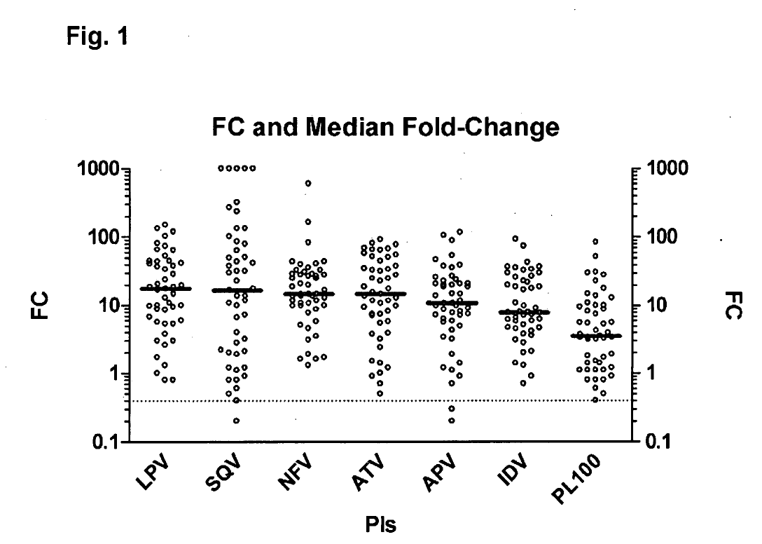

The results presented in the above Table 6 indicates the fold-change (FC) in IC₅₀ vs the reference strain (NL-4.3). The dark cells indicate a FC≦10, the grey cells indicate FC values of 2.5≦FC<10 and the white cells indicate a FC value of; 0≦FC<2.5. The results of cross-resistance profile are further illustrated in the graph of FIG. 1.

Table 7 summarizes the phenotypic susceptibility results obtained against the 63 diverse, multi-PI-resistant strains mentioned above.

TABLE 7

| | ATV | APV | IDV | LPV | NFV | SQV | PL-100 |
|---|---|---|---|---|---|---|---|
| Median FC | 15.6 | 9.7 | 8.1 | 17.9 | 15 | 23.2 | 3.6 |
| Mean FC | 25.7 | 18.5 | 16.5 | 31.3 | 31.7 | 85 | 8.7 |
| % FC < 2.5 | 16 | 14 | 13 | 10 | 10 | 27 | 37 |
| % FC < 10 | 38 | 52 | 54 | 37 | 27 | 40 | 76 |
| % FC > 50 | 22 | 8 | 5 | 19 | 10 | 37 | 3 |

In the cross-resistance profiling studies against the 63 resistant strains mentioned herein, EC₉₅ of PL-100 was determined against each strain. The median EC₉₅ represents the concentration required to inhibit 95% of the viral replication of 50% diverse drug-resistant HIV-1 variants tested. Table 8 illustrates the percentage of resistant strains tested with protein-binding adjusted EC₉₅s≦200 ng/ml. The resistant strains are grouped by the number of primary mutations they have.

TABLE 8

| No. of primary PI | % viral strains with |
|---|---|
| mutations | EC₉₅s ≦ 200 ng/ml |
| 0 | 100 |
| 1 | 100 |
| 2 | 36 |
| 3 | 23 |
| 4 | 0 |
| 5-6 | 0 |

The EC₉₅ of PL-100 was determined against each of the 63 resistant strains indicated herein. Table 9 illustrates the percentage of resistant strains tested with protein-binding adjusted EC₉₅s≦630 ng/ml. The resistant strains are grouped by the number of primary mutations they have.

TABLE 9

| No. of primary PI | % viral strains with |
|---|---|
| mutations | EC₉₅s ≦ 630 ng/ml |
| 0 | 100 |
| 1 | 100 |
| 2 | 82 |
| 3 | 55 |
| 4 | 29 |
| 5-6 | 0 |

Results presented herein indicate that PL-100 has a favourable cross-resistance profile against several HIV isolates.

### Example 13

### Bioavailability of Compounds

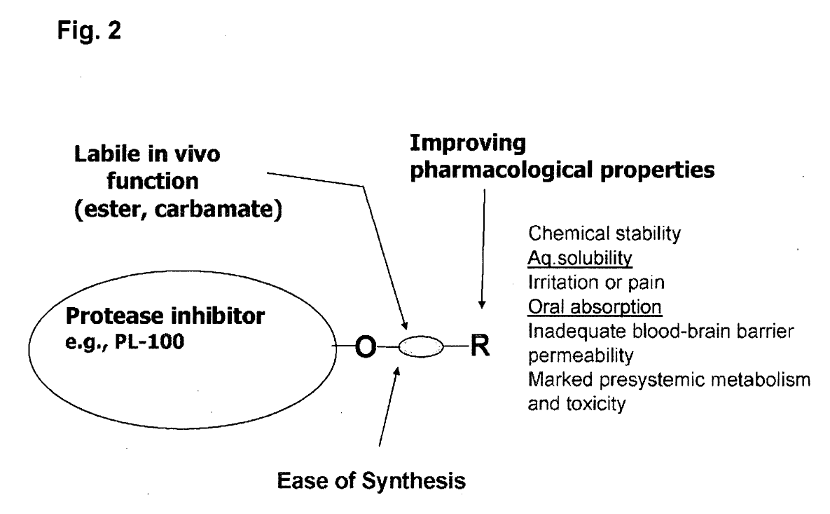

In order to improve the chemical stability, aqueous solubility of the active ingredients, various Lysine-based compounds, such as PL-100 derivatives (PL-461, PL-462, etc.) were designed, synthesized and purified (FIG. 2, Table 10). The active ingredient has been shown to be efficient against an HIV-1 aspartyl protease (U.S. Pat. No. 6,632,816). Also as mentioned herein, the active ingredients present potent antiviral activity when tested on non-mutated HIV-1 viral strain (NL4.3 as the wild type virus) as well as several mutant strains.

Various PL-100 precursors were thus developed and tested. Examples of representative PL-100 precursors are illustrated in Table 10. PL-100 and PL-337 have been chosen herein as representative active ingredient among those disclosed in U.S. Pat. No. 6,632,816 to Stranix et al. for improving pharmacokinetics of these potent inhibitors.

The compounds listed in Table 10 were prepared by following scheme 1, 1A, 2, 3, 4 or 5; and more particularly as described in each example listed above. The numbers of the compounds listed in Table 10 (Ex. No.) corresponds to the example numbers presented above.

TABLE 10
Structures of exemplary lysine based compounds and active
ingredients encompassed by the present invention

| | |
|---|---|
| | I |
| 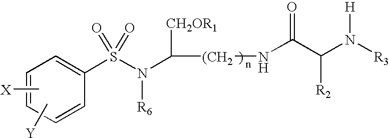 | |

| | | | | | | | | | D, L, |
|---|---|---|---|---|---|---|---|---|---|
| | | | | | | | | | DL |
| Ex. No | | | | | | | | | R, S, |
| (PL-#) | X | Y | n | R₁ | R₂ | R₃ | R₆ | X′/Y′ | RS |
| 1 (PL-461) | 4-NH₂ | H | 4 | (HO)₂P(O) | (C₆H₅)₂CH | CH₃O—CO | iso-butyl | H/H | S, S |
| 2 (PL-462) | 4-NH₂ | H | 4 | (NaO)₂P(O) | (C₆H₅)₂CH | CH₃O—CO | iso-butyl | H/H | S, S |
| 3 (PL-507) | 4-NH₂ | H | 4 | (HO)₂P(O) | Naphthyl-2-CH₂ | CH₃O—CO | iso-butyl | H/H | S, S |
| 4 (PL-498) | 4-NH₂ | H | 4 | (HO)₂P(O) | Naphthyl-1-CH₂ | 4-morpholine- | iso-butyl | H/H | S, S |
| | | | | | | CO | | | |
| 5 (PL-504) | 4-NH₂ | H | 4 | (HO)₂P(O) | (C₆H₅)₂CH | CH₃CO | iso-butyl | H/H | S, S |
| 6 (PL-515) | 4-NH₂ | 3-F | 4 | (HO)₂P(O) | (C₆H₅)₂CH | CH₃O—CO | iso-butyl | H/H | S, S |
| 7 (PL-521) | 4-NH₂ | H | 4 | CH₃CO | (C₆H₅)₂CH | CH₃O—CO | iso-butyl | H/H | S, S |
| 8 (PL-520) | 4-NH₂ | H | 4 | 3-Pyridyl-CO | (C₆H₅)₂CH | CH₃O—CO | iso-butyl | H/H | S, S |
| 9 (PL-534) | 4-NH₂ | H | 4 | (CH₃)₂NCH₂CO | (C₆H₅)₂CH | CH₃O—CO | iso-butyl | H/H | S, S |
| 10 (PL-530) | 4-NH₂ | H | 4 | (CH₃)₂CHCH(NH₂)CO | (C₆H₅)₂CH | CH₃O—CO | iso-butyl | H/H | S, S |
| 11 (PL-100) | 4-NH₂ | H | 4 | H | (C₆H₅)₂CH | CH₃O—CO | iso-butyl | H/H | |
| 12 (PL-337) | 4-NH₂ | 3-F | 4 | H | C₆H₅)₂CH | CH₃O—CO | iso-butyl | H/H | |

To assess the extent of in vivo cleavage of the phosphate group from the putative compounds, PL-100, PL-462 (based on PL-100), PL-337 and PL-515 (based on PL-337) compounds were administered po (per os, (i.e., by mouth)) (50 mg/kg) to male Sprague-Dawley rats and their plasma concentration measured at different time intervals post-administration.

All test articles (PL-100, PL-462, PL-337 and PL-515) were prepared in different vehicle at the final concentration of 25 mg/mL. The vehicle composition is as follows: (1) 20% ethanol; 50% propylene glycol; 0.05% w/v Tween 20 and water (Mix); (2) PBS buffer (PBS).

Test articles were administered to male Sprague-Dawley rats at a single oral dose of 50 mg/kg. Each article was tested in three rats. Blood samples (0.2-0.3 mL) were collected at the post-dose time of 10, 20, 40, 60, 120, 180 and 360 minutes. The harvested blood was centrifuged to isolate plasma. The resulting plasma was separated and stored at −70° C.

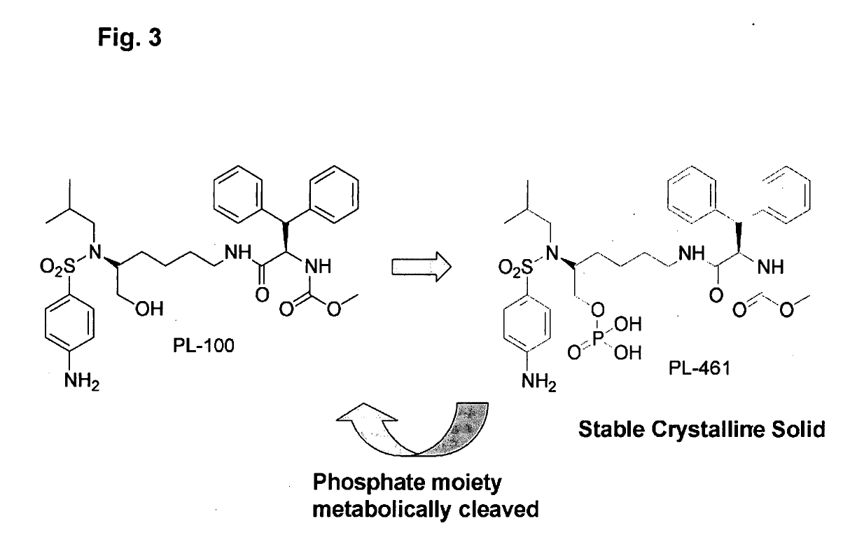

As indicated above, when the Lysine-based compounds described herein are administered in vivo, the active ingredient is released and it may therefore inhibit the protease of retroviruses. For example, as illustrated in FIG. 3 when PL-461 (or PL-462) is administered in vivo, the phosphate moiety (in the case of PL-461) is metabolically cleaved to generate the active ingredient; PL-100. Similarly when PL-515 is administered in vivo, the phosphate moiety is cleaved to generate PL-337.

Plasma samples together with standards and quality control samples were treated to precipitate proteins, then analyzed by HPLC-MS, for the presence of PL-462, PL-100, PL-515 and PL-337.

TABLE 11

| | PL-462 | PL-100 | PL-515 | PL-337 |
|---|---|---|---|---|
| Compound | (Ex. No. 2) | (Ex. No. 1-F) | (Ex. No. 13) | (Ex. No. 13-A) |
| Vehicle | PBS | Mix | PBS | Mix |
| Number of | 3 | 3 | 3 | 3 |
| rats | | | | |
| Dose (mg/Kg) | 50 po | 50 po | 50 po | 50 po |
| AUC | 0.816 ± 0.295 | 0.675 ± 0.171 | 1.075 ± 0.625 | 1.180 ± 0.196 |
| (μg/hr * ml) | (PL-100, detected) | | (PL-337, detected) | |
| Cmax (nM) | 330 ± 109 | 498 ± 203 | 545 ± 215 | 681 ± 131 |
| Tmax (min) | 93 ± 60 | 40 ± 16 | 87 ± 60 | 60 ± 15 |

In Table 11 above:
50 mg/Kg PL-462 = 43 mg/Kg PL-100
50 mg/Kg PL-515 = 43 mg/Kg PL-337

The results demonstrate that PL-462 and PL-515 compounds may be delivered orally in aqueous solutions. None of the PL-462 and PL-515 compounds, delivered as aqueous solutions, are detected in the blood samples, which suggests rapid metabolism to PL-100 and PL-337 the parent drugs.

Aqueous dosing of PL-462 and PL-515 solutions showed equivalent to slightly superior delivery of PL-100 and PL-337 compared to non-aqueous formulations of PL-100 and PL-337.

Based on these results, all the phosphorylated compounds described in the present invention will demonstrate similar pharmacokinetic properties.

Partition coefficient (LogP) of selected compounds and the corresponding HIV protease inhibitors (drug) are as follow:

TABLE 12

| | | Corresponding | |
|---|---|---|---|
| Compounds | LogP | drugs | LogP |
| PL-461 (or PL-462) | −1.2 | PL-100 | 3.6 |
| PL-515 | −0.75 | PL-337 | 3.8 |

The LogP were measured in a standard fashion by dissolving 1 mg of compound in 0.8 mL of each octanol and phosphate buffer pH 7.4 (0.04 M KHPO₄). The concentration of the compounds in the phases was detected by LC-MS. This test demonstrates the solubility of the compounds at physiological pH. The LogP obtained show that the compounds are highly soluble as compared to the corresponding drugs.

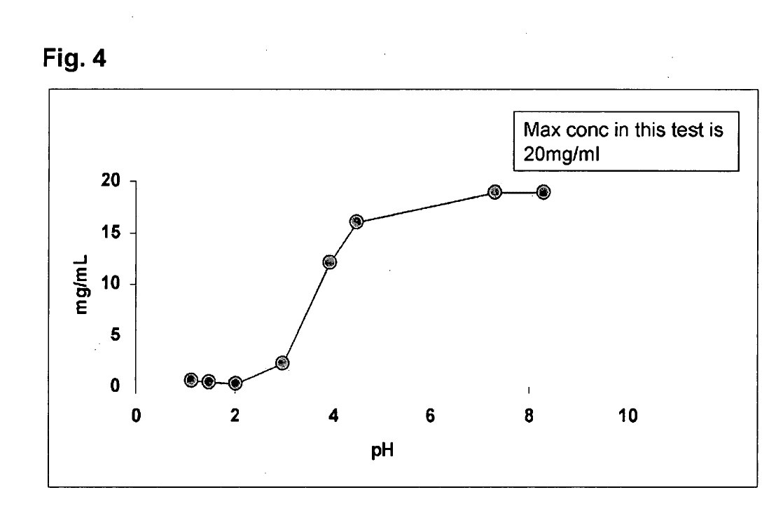

Results of solubility experiments illustrated at FIG. 4 indicates that PL-461 also possesses a moderate water solubility in acidic conditions and that the solubility increases dramatically when pH>4.5. Water solubility of PL-100 and PL-461 at pH 7.5 is 0.079 and 145 mg/ml for PL-100 and PL-461, respectively.

The phosphorylated version of PL-100; namely PL-461, was selected for further evaluation due to its stability, solubility and oral bioavailability.

Materials and Methods-Pharmacokinetics Studies

Several studies were conducted in order to test the pharmacokinetic profile of the Lysine-based compounds described herein either alone or in combination with CYP450 inhibitors.

A first study (study 1 #141690) was conducted to evaluate the pharmacokinetic profile of PL-100 and PL-461 after a single dose oral administration in combination with ritonavir and two other studies (study 2 #143656 and study 3 #144536) were conducted to evaluate the pharmacokinetic profile of PL-461 after a single dose oral administration in combination with ritonavir.

Animal tested are Sprague Dawley rats (species Rattus norvegicus) of 6-7 weeks old at the start of dosing and were obtained from Charles River (Montreal, Canada). The study usually comprises about 6 animals per groups.

Twenty four hours prior to the initiation of the study, rats were randomly selected into 6 groups, and each group had 6 rats (3 rats for each cage). The rats were fasted 3 h. before dosing and 3 h. after dosing.

The formulations are prepared as indicated below. The solutions are stable at room temperature over a week and are covered with aluminum foil to avoid lights. The mixture contains Ethanol, Propylene Glycol, Tween 20 and Water for Injection (20%, 50%, 0.1% and 30%, v/v/v/v). The pH of all solutions has been adjusted to 7.5. The dosing volume for all formulations is 2 mL per kg body weight.

Blood sample was collected at 15 min, 30 min, 45 min, 1 h, 2 h, 3 h, 6 h, 12 h, 24 h after the dosing. Blood samples were centrifuged to separate the plasma and separated plasma samples were placed into cryovials and frozen at −80° C. for further analysis. Plasma samples together with standards and quality control samples were treated by protein precipitation and then analyzed by HPLC-MS.

Protein Precipitation

Briefly, protein precipitation was performed on aliquot of 50.0 mL blank plasma (for standards and quality control samples (QCs)) or PK plasma. 5.00 μL of spiking solution was added for standards and QCs or acetonitrile/DMSO=1/1 for PK samples.

The samples were mixed by vortexing for 10 seconds. 150 uL of internal standard working solution (1.00 ug/mL PL-459-01 in acetonitrile) was added. Samples were again vortexed for 10 seconds and sonicated for 5 mins. The samples were centrifuged for 10 mins at 10,000 rpm and 100 uL of the supernatant was transferred into an insert. 100 uL of deionized water (0.2% FA) was added and sample vortexed. Finally, 20 uL was injected in HPLC.

Instrument Conditions for HPLC

HPLC:
Column: Zorbax SB-C18, 2.1 × 50 mm, 5 μm
Mobile phase: aqueous (Aq) = 0.1% formic acid in deionized water
Organic (Org) = 0.1% formic acid in acetonitrile
Gradient: From 30% to 90% of Org in 5 mins, then return to 30% of Org
in 1 min
and equilibres 4 mins
Flow rate: 0.30 mL/min
Run time: 10 mins
Retention time: 5.9 min for PL-100 and PL-459 (IS), 4.6 min for PL-461,
6.3 min for
ritonavir
MSD:
Source: ESI
Peakwidth: 0.07 min
Polarity: Positive
Acquisition time: 2.0-7.0 mins
Acquisition mode: SIM, MH+, 625.3 for PL-100; 630.3 for PL-459 (IS),
705.3 for PL-
461, 721.3 for ritonavir
Fragmentor: 100
Gain: 1.5
Resolution: High
Spray chamber:

| | |
|---|---|
| Gas temperature: 325° C. | Drying gas: 12.0 L/min |
| Nebulizer pressure: 55 psig | Vcap: 4000 V |

Bioavailability Calculations

$$Bioavailability of PL 461 = \frac{( {AUC}_{0 - 24 h} - p . o . )}{( {AUC}_{0 - 24 h} - iv . )} \cdot \frac{Dose - iv}{Dose - po} \cdot \frac{M . W . - PL 461}{M . W . - PL 100} \cdot 100 \%$$

$$Bioavailability of PL 100 = \frac{( {AUC}_{0 - 24 h} - p . o . )}{( {AUC}_{0 - 24 h} - iv . )} \cdot \frac{Dose - iv}{Dose - po} \cdot 100 \%$$

$$Note \text{:}$$

$$Molecular Weight of PL 461 = 705$$

$$Molecular Weight of PL 100 = 624$$

Test Articles for Studies 1, 2 and 3

Unless specifically mentioned, female rats were used.

Test articles of study 1

Name:
PL-100-06
Batch/Lot No.: 06
Storage Conditions: Room Temperature (15-30° C.)
Name:
PL-461-05
Batch/Lot No.: 05 (ZL-177-091404)
Storage Conditions: Room Temperature (15-30° C.)
Name:
Ritonavir
Batch/Lot No.: 749592E22
Storage Conditions: Room Temperature (15-30° C.)

Formulation Used in Study 1

Formulation 1:

PL-100-06 (30 mg/kg) concentration is 15 mg/mL

PL-100-06; 90.0 mg dissolved in 6.00 mL Mix

Formulation 2:

PL-100-06 (30 mg/kg) concentration is 15 mg/mL and Ritonavir (10 mg/kg) concentration is 5 mg/mL

PL-100-06; 90.0 mg+30 mg RTV dissolved in 6.00 mL Mix

Formulation 3:

PL-100-06 (30 mg/kg) concentration is 15 mg/mL and Ritonavir (30 mg/kg) concentration is 15 mg/mL

PL-100-06; 90.0 mg+90 mg RTV dissolved in 6.00 mL Mix

Formulation 4:

PL-461-05 (30 mg/kg) concentration is 15 mg/mL

PL-461-05; 90.0 mg dissolved in 6.00 mL Mix, pH 7.0

Formulation 5:

PL-461-05 (30 mg/kg) concentration is 15 mg/mL and Ritonavir (10 mg/kg) concentration is 5 mg/mL

PL-461-05; 90.0 mg+30 mg RTV dissolved in 6.00 mL Mix, pH 7.0

Formulation 6:

PL-461-05 (30 mg/kg) concentration is 15 mg/mL and Ritonavir (30 mg/kg) concentration is 15 mg/mL

PL-461-05; 90.0 mg+90 mg RTV dissolved in 6.00 mL Mix, pH 7.0

Test articles of study 2

Name:
PL-461-05
Batch/Lot No.: 05 (ZL-7-177-091404)
Storage Conditions: Room Temperature (15-30° C.)
Name:
Ritonavir
Batch/Lot No.: 749592E22
Storage Conditions: Room Temperature (15-30° C.)

Formulation Used in Study 2

Formulation 1:

PL-461-05 (29.93 mg/kg) concentration is 14.97 mg/mL

PL-461-05; 89.8 mg dissolved in 6.00 mL Mix

Formulation 2:

PL-461-05 (30.2 mg/kg) concentration is 15.10 mg/mL and Ritonavir (10 mg/kg) concentration is 5 mg/mL

PL-461-05; 90.6 mg+30 mg RTV dissolved in 6.00 mL Mix

Formulation 3:

PL-461-05 (29.43 mg/kg) concentration is 14.72 mg/mL and Ritonavir (5.10 mg/kg) concentration is 2.55 mg/mL

PL-461-05; 88.3 mg+15.3 mg RTV dissolved in 6.00 mL Mix

Formulation 4:

PL-461-05 (51.27 mg/kg) concentration is 25.63 mg/mL

PL-461-05; 153.8 mg dissolved in 6.00 mL Mix, pH 7.0

Formulation 5:

PL-461-05 (50.03 mg/kg) concentration is 25.02 mg/mL and Ritonavir (17.03 mg/kg) concentration is 8.52 mg/mL

PL-461-05; 150.1 mg+51.1 mg RTV dissolved in 6.00 mL Mix, pH 7.0

Formulation 6:

PL-461-05 (49.33 mg/kg) concentration is 24.67 mg/mL and Ritonavir (8.03 mg/kg) concentration is 4.02 mg/mL

PL-461-05; 148.0 mg+24.1 mg RTV dissolved in 6.00 mL Mix, pH 7.0

Test articles of study 3

| | |
|---|---|
| Name: | PL-461-06 |
| Batch/Lot No.: | 06 (SAP-8-57-102604) |
| Storage Conditions: | Room Temperature (15-30° C.) |
| Name: | Ritonavir (RTV) |
| Batch/Lot No.: | 749592E22 |
| Storage Conditions: | Room Temperature (15-30° C.) |

Formulation Used in Study 3

Formulation 1:

PL-461-06 (30 mg/mL) dose is 60 mg/kg

PL-461-06; 180.0 mg dissolved in 6.00 mL Mix

Formulation 2:

PL-461-06 (15 mg/mL) dose is 30 mg/kg and Ritonavir (5 mg/mL) dose is 10 mg/kg

PL-461-06; 90.0 mg+30 mg RTV dissolved in 6.00 mL Mix

Formulation 3:

PL-461-06 (50 mg/mL) dose is 100 mg/kg and Ritonavir (5 mg/mL) dose is 10 mg/kg

PL-461-06; 300.0 mg+30 mg RTV dissolved in 6.00 mL Mix

Formulation 4:

PL-461-06 (50 mg/mL) dose is 100 mg/kg

PL-461-06; 300.0 mg dissolved in 6.00 mL Mix

Formulation 5:

PL-461-06 (30 mg/mL) dose is 60 mg/kg and Ritonavir (5 mg/mL) dose is 10 mg/kg

PL-461-06; 180.0 mg+30 mg RTV dissolved in 6.00 mL Mix

Formulation 6:

PL-461-06 (50 mg/mL) dose is 100 mg/kg and Ritonavir (8.33 mg/mL) dose is 16.67 mg/kg

PL-461-06; 300 mg+50 mg RTV dissolved in 6.00 mL Mix

Test articles of study 4

| | |
|---|---|
| PL-461-06 | |
| Batch/Lot No.: | 06 (SAP-8-57-102604) |
| Storage Conditions: | Room Temperature (15-30° C.) |
| Ritonavir (RTV) | |
| Batch/Lot No.: | 21-802AW21 |
| Storage Conditions: | Room Temperature (15-30° C.) |
| Manufacturer: | Abbott Laboratories, Limited |

Preparation used in study 4
PL-461-06 Preparation 1

| | |
|---|---|
| Test Articles: | PL-461 (20 mg/mL) |
| Vehicle: | Water for injection |
| pH: | 6.0 |
| Appearance: | Pale yellow solution |
| Storage Condition: | Room temperature (15-30° C.) |

PL-461-06 Preparation 2

| | |
|---|---|
| Test Articles: | PL-461 (60 mg/mL) |
| Vehicle: | Water for injection |
| pH: | 6.0 |
| Appearance: | Pale yellow solution |
| Storage Condition: | Room temperature (15-30° C.) |

PL-461-06 Preparation 3

| | |
|---|---|
| Test Articles: | PL-461 (200 mg/mL) |
| Vehicle: | Water for injection |
| pH: | 6.0 |
| Appearance: | Yellow solution |
| Storage Condition: | Room temperature (15-30° C.) |

Ritonavir Solution (16 mq/mL)

2 mL of the Ritonavir Solution, Norvir, (80 mg/mL) is diluted with 8 mL mixture containing 60% Propylene Glycol and 40% Water. The final concentration of Ritonavir is 16.0 mg/mL.

### Example 14

### Pharmacokinetics of PL-461

The pharmacokinetics (PK) of PL-461 was tested and compared with that of the original active ingredient.

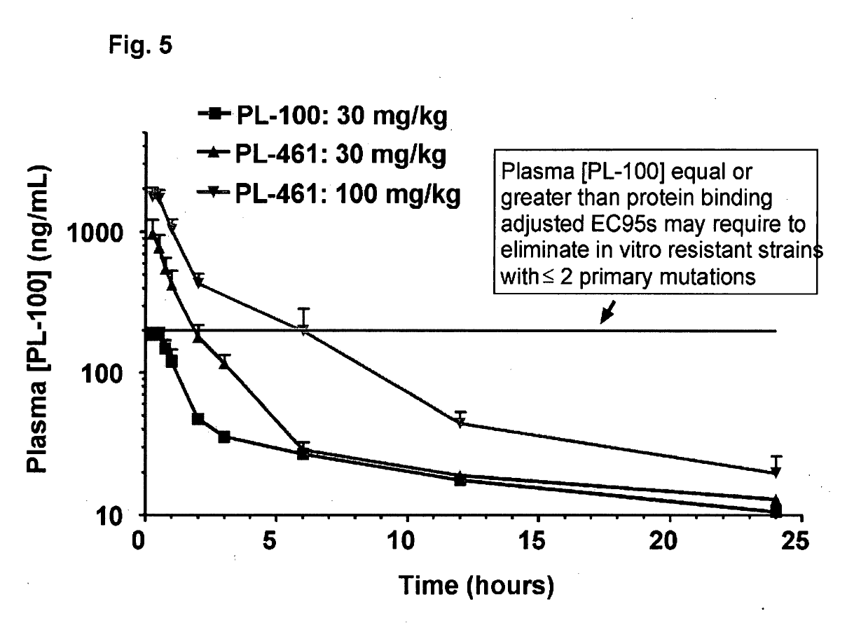

The PK profile was obtained by administration of the test drugs; PL-100 and PL-461 in rats. PL-100 or PL-461 was administered orally at doses indicated in FIG. 5. Each time point in the figure represents the average plasma [PL-100] (ng/ml) of 6 female rats at a given dose.

The results indicate that the absolute oral bioavailability for PL-100 and PL-461 at 30 mg/kg is 8.7 and 23%, respectively. The bioavailability of PL-461 at 100 mg/kg is 23%.

The horizontal line in FIG. 5 represents plasma [PL-100] of 200 ng/ml. The PK profile of PL-461 shows that the time (t)>200 ng/ml is approximately 6 hours in rats at 100 mg/kg, suggesting PL-461 has a potential as a twice daily drug to maintain such plasma [PL-100] in man at an equivalent dose.

The pharmacokinetic parameters of PL-100 and PL-461 in rats** was compared. These results are summarized in Table 13 below.

TABLE 13

| | Dose | | λz | T½ | Tmax | Cmax | Tlast | Clast | AUClast | AUCinf | Vz_F | Cl_F |
|---|---|---|---|---|---|---|---|---|---|---|---|---|
| | mg/kg | Route | (1/hr) | (hr) | (hr) | (ng/ml) | (hr) | (ng/ml) | (hr ng/ml) | (hr ng/ml) | (L/kg) | (L/hr/kg) |
| PL-100* | 5 | i.v. | 0.52 | 1.2 | 0.1 | 1804 | 6 | 44 | 1369 | 1434 | 6.1 | 3.5 |
| PL-100* | 50 | p.o. | 0.09 | 8.4 | 0.4 | 457 | 24 | 15 | 1101 | 1284 | 549 | 44 |
| PL-461* | 50 | p.o. | 0.10 | 9.4 | 0.5 | 1216 | 24 | 13 | 2226 | 2416 | 282 | 23 |

In Table 13, the abbreviations are as follows:

| | |
|---|---|
| λz | elimination rate constant |
| t½ | plasma elimination half-life |
| Tmax | time of maximum concentration |
| Cmax | maximum concentration |
| Tlast | time of last measurable concentration |
| Clast | last measurable concentration |
| AUClast | area under the plasma conc-time curve from zero to the last measurable |
| | concentration |
| AUCinf | area under the plasma concentration-time curve from zero extrapolated to |
| | infinity |
| Vz_F | apparent volume of distribution |
| Cl_F | apparent oral clearance |

*Each treatment group had at least 6 female rats.
**PK analysis was done using the non-compartmental method with WinNonLin Professional
(version 4.0). AUC was calculated using the linear-up/log-down method.
Results presented in FIG. 5 and in Table 13 indicate that PL-461 has improved PK over PL-
100.

### Example 15

### Methods and Compositions for Improving the Pharmacokinetics of Active Ingredients and Lysine-Based Compounds

The results presented herein indicate that although the active ingredients (e.g., PL-100, etc.) and the Lysine-based compounds (e.g., PL-461, PL-462, etc.) are good anti-viral compounds, the pharmacokinetics may still be improved.

Further experiments were therefore conducted to that effect by administering PL-100 or PL-461 with a drug able to inhibit cytochrome P450 monooxygenase (CYP450) (i.e., a CYP450 inhibitor) in order to test whether this type of inhibitor may increase the pharmacokinetics of compounds described herein.

Ritonavir, quinidine, ketoconazole and sulfaphenazole were chosen among candidate CYP450 inhibitors and the pharmacokinetics of PL-461 and CYP450 inhibitors combinations was compared with the pharmacokinetics of other PI and CYP450 inhibitors combination. The effect of CYP450 inhibitors on the metabolism of HIV-1 anti-protease was tested in human liver microsomes.

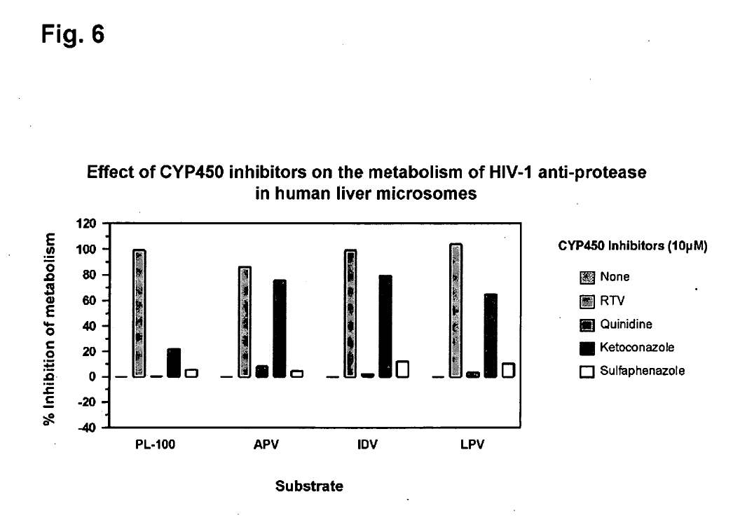

Although, ketoconazole and sulfaphenazole are able to increase the PK of PL-100, results of FIG. 6 indicate that the most effective combination is that of PL-100 and ritonavir.

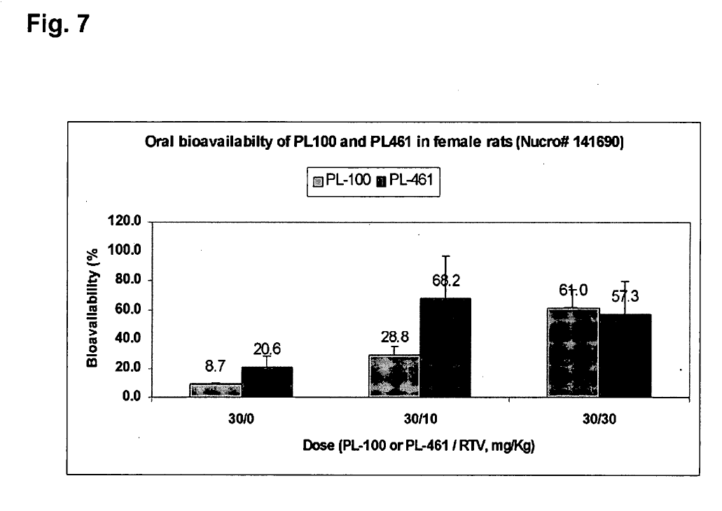

Results presented in FIG. 7 and in Table 14 illustrate the bioavailability of PL-100 or PL-461 when administered in combination with a CYP450 inhibitor.

TABLE 14
Oral bioavailability of PL-100 and PL-461 in female rats (# 141690)

| Group | Test | Dose* | Dose** | | | | | | | | | | | | |
|---|---|---|---|---|---|---|---|---|---|---|---|---|---|---|---|
| No. | article | (mg/Kg) | (mg/Kg) | Absolute Bioavailability*** (%) | | | | | | Ave. | SD | CV (%) | Max | Min | Median |
| 1 | PL100/RTV | 30/0 | 30/0 | 8.5 | 8.4 | 8.5 | 9.0 | 7.2 | 10.7 | 8.7 | 1.1 | 12.8 | 10.7 | 7.2 | 8.5 |
| 2 | | 30/10 | 33/10 | 36.3 | 25.0 | 24.8 | 31.3 | 21.9 | 33.7 | 28.8 | 6.3 | 19.9 | 36.3 | 21.9 | 28.1 |
| 3 | | 30/30 | 32/30 | 70.0 | 54.6 | 75.3 | 54.8 | 70.2 | 41.2 | 61.0 | 13.0 | 21.3 | 75.3 | 41.2 | 62.4 |
| 4 | PL461/RTV | 30/0 | 35/0 | 15.9 | 33.6 | 13.0 | 14.9 | 22.3 | 23.8 | 20.6 | 7.7 | 37.3 | 33.6 | 13.0 | 19.1 |
| 5 | | 30/10 | 34/11 | 80.7 | 45.1 | 105.1 | 94.3 | 47.7 | 36.5 | 68.2 | 28.8 | 42.3 | 105.1 | 36.5 | 64.2 |
| 6 | | 30/30 | 34/34 | 32.7 | 93.8 | 42.1 | 64.9 | 66.7 | 43.5 | 57.3 | 22.4 | 39.1 | 93.8 | 32.7 | 54.2 |

*Intended dose by protocol
**Corrected dose by QC results.
***Bioavailability was calculated from corrected dose.

These results indicate that ritonavir (RTV: a CYP450 inhibitor) increases the bioavailability of PL-100 and that of PL-461 as well.

Further CYP450 inhibitor efficient at increasing the pharmacokinetics of the active ingredients or the Lysine-based compounds (referred here as “compounds”) described herein may be identified by co-administering a putative CYP450 inhibitor and a desired compound (one or more compounds) in an assay (e.g., microsome assay or in vivo studies) described herein and to measure the amount of residual compound (active ingredient) still remaining after various time points following administration (incubation). A CYP450 inhibitor which increases the bioavailability of the compound (active ingredient) compared to the bioavailability measured without the putative CYP450 inhibitor would be found efficient for the purpose of improving the pharmacokinetics of the compound.

In study 4, ritonavir was first administered and PL-1461 was administered 15 or 30 minutes later. The result obtained using this administration scheme (Table 15) indicates that this method is as efficient at increasing the pharmacokinetics of the Lysine-based compound disclosed herein as the method of studies 1-3.

TABLE 15

| | | | Conc. of | | | |
|---|---|---|---|---|---|---|
| | | | Test | | Dosing | Number of |
| | | | Article | Route of | Volume | Animals per |
| Group | Test Article | Dose (mg/kg) | (mg/mL) | Administration | (mL/kg) | Group |
| 1 | PL-461-06 | 100 | 20 | Oral | 5 | 3 male/ |
| | | | | | | 3 female |
| 2 | PL-461-06 | 300 | 60 | Oral | 5 | 3 male/ |
| | | | | | | 3 female |
| 3 | PL-461-06 | 1000 | 200 | Oral | 5 | 3 male/ |
| | | | | | | 3 female |
| 4 | RTV | 16 | 16 | Oral | 1 | 3 male/ |
| | PL-461-06 | 100 | 20 | Oral | 5 | 3 female |
| 5 | RTV | 16 | 16 | Oral | 1 | 3 male/ |
| | PL-461-06 | 100 | 20 | Oral | 5 | 3 female |
| 6 | RTV | 16 | 16 | Oral | 1 | 3 male/ |
| | PL-461-06 | 100 | 20 | Oral | 5 | 3 female |

### Example 16

### Pharmacokinetics of Lysine-Based Compounds and CYP450 Inhibitors Combination

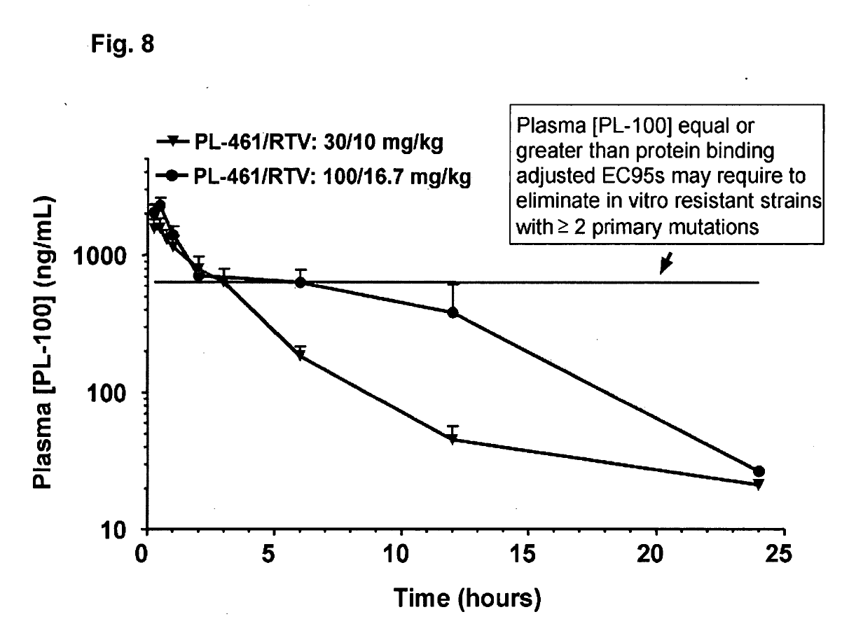

In studies 1, 2 and 3 mentioned above, PL-461 and RTV were co-administered orally at doses indicated in FIG. 8.

Each time point in FIG. 8 represents the average plasma concentration of PL-100 (ng/ml) of 6 female rats at a given dose. The horizontal line in FIG. 8 represents plasma concentration of PL-100 of 630 ng/ml. The PK profile shows that the time (t)>630 ng/ml is approximately 6 hours in rats at 100 mg/kg PL-461 and 16.7 mg/kg RTV, suggesting that PL-461 has a potential as a twice daily drug to maintain such plasma concentration of PL-100 in man at an equivalent dose.

### Example 17

### Optimization of Lysine-Based Compounds vs CYP450 Inhibitor Ratio

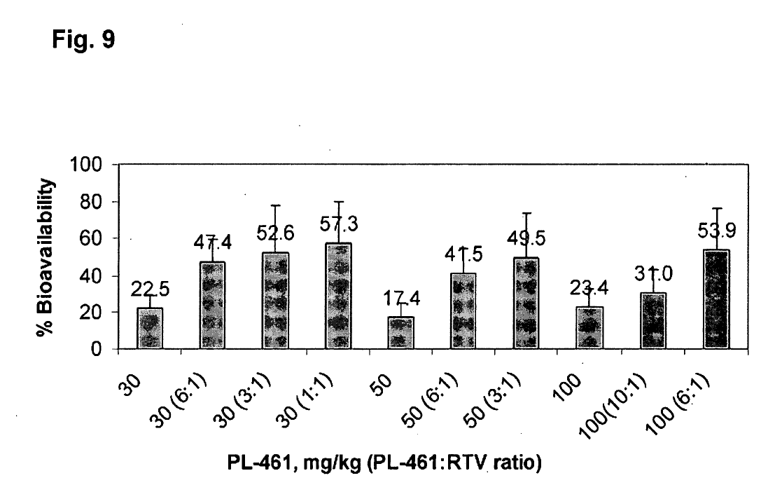

The absolute oral bioavailability of PL-461 was determined under various conditions indicated in FIG. 9. More particularly, the concentration of PL-461 and the proportion of PL-461 compared to CYP450 was varied. Results presented herein indicate that sufficient oral bioavailability, relative to protein binding adjusted EC₉₅ against resistant strains may be achieved when boosted at a ratio of 6 (PL-461) to 1 (RTV), although other ratios may also successfully be used as indicated in Table 16 below.

TABLE 16
Oral bioavailability of PL-461 in rats with and without ritonavir (#141690, #143656, #144536)

| Dose level | Ratio of dose | Dose* (mg/kg) | | | | | | | | | | | | |
|---|---|---|---|---|---|---|---|---|---|---|---|---|---|---|
| of PL461 | (PL461/RTV) | (PL461/RTV) | Absolute Bioavailability (%)** | | | | | | Ave. | SD | CV (%) | Max | Min | Median |
| 30 mg/kg | N/A*** | 35/0 | 15.9 | 33.6 | 13.0 | 14.9 | 22.3 | 23.8 | 22.5 | 6.9 | 30.7 | 33.6 | 13.0 | 23.8 |
| | | 27/0 | 30.1 | 24.0 | | 24.5 | | | | | | | | |
| | 6/1 | 26/3.5 | 42.2 | 37.6 | | 45.1 | 65.5 | | 47.4 | 12.3 | 26.0 | 65.5 | 37.6 | 43.7 |
| | 3/1 | 34/11 | 80.7 | 45.1 | 105.1 | 94.3 | 47.7 | 36.5 | 52.6 | 24.5 | 46.6 | 105.1 | 26.5 | 43.9 |
| | | 26/9.2 | 62.1 | 45.3 | 26.5 | 38.5 | 42.6 | 34.7 | | | | | | |
| | | 26/8.4 | 94.7 | 37.1 | 32.9 | 28.6 | 54.9 | 39.4 | | | | | | |
| | 1/1 | 34/34 | 32.7 | 93.8 | 42.1 | 64.9 | 66.7 | 43.5 | 57.3 | 22.4 | 39.1 | 93.8 | 32.7 | 54.2 |
| 50 mg/kg | N/A*** | 48/0 | 11.1 | 20.3 | 9.2 | 23.3 | | 31.3 | 17.4 | 7.7 | 44.6 | 31.3 | 7.1 | 14.7 |
| | | 51/0 | 12.0 | 7.1 | 14.7 | 24.4 | 13.4 | 24.4 | | | | | | |
| | 6/1 | 55/11 | 34.9 | 51.7 | 33.9 | 58.4 | 46.0 | 24.1 | 41.5 | 12.8 | 30.7 | 58.4 | 24.1 | 40.5 |
| | 3/1 | 45/15 | 49.0 | 64.5 | 41.9 | 42.3 | 13.8 | 85.2 | 49.5 | 24.0 | 48.6 | 85.2 | 13.8 | 45.7 |
| 100 mg/Kg | N/A*** | 84/0 | 13.6 | 37.1 | 25.3 | 25.6 | 15.2 | | 23.4 | 9.5 | 40.6 | 37.1 | 13.6 | 25.3 |
| | 10/1 | 85/9.2 | 25.2 | 20.1 | 40.0 | 20.3 | 28.9 | 51.4 | 31.0 | 12.4 | 40.0 | 51.4 | 20.1 | 27.1 |
| | 6/1 | 89/17 | 91.2 | 47.0 | 39.1 | 56.7 | 35.6 | | 53.9 | 22.4 | 41.5 | 91.2 | 35.6 | 47.0 |

*Corrected dose by QC results.
**Bioavailability was calculated by corrected dose.
***No Ritonavir

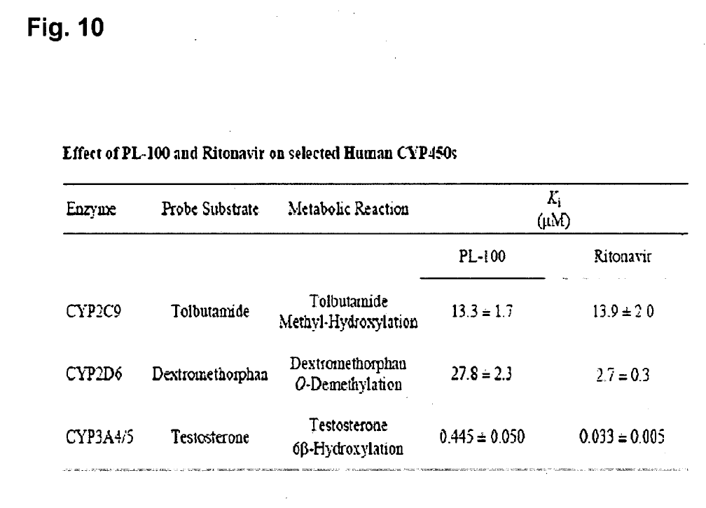

Finally FIG. 10 indicates that PL-100 inhibits CYP3A4/5 as does RTV. RTV's higher Ki (about 10-fold higher) confirms that it is able to act as a boosting agent for PL-100.

It was shown herein that PL-100 is a potent, specific and non-cytotoxic novel PI and that it has a favorable cross-resistance pattern compared to all approved Pls.

PL-461, a precursor of PL-100, is >1800-fold more water soluble than PL-100 and has a 2 to 3-fold improved oral bioavailability over PL-100. PL-461 has a potential as a novel PI for the treatment of patients infected with PI-resistant HIV strains bearing, for example, two or less primary mutations. PL-461, when combined with a CYP450 inhibitor such as, for example, ritonavir, has a great potential as a novel PI for the treatment of patients infected with PI-resistant HIV strains bearing mutations (e.g., two or more primary mutations). The ratio of PL-461 to ritonavir required for boosting has been found herein adequate for in vivo administration.

On the basis of PL-100 cross-resistance data and the pharmacokinetics results, it is expected that equivalent doses of PL-461 in man will effectively inhibit replication of protease-resistant HIV strains. Moreover, these data also suggests that PL-461 may be administered, for example, using a convenient twice-daily dosing regimen.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

We claim:

1. A pharmaceutical composition comprising:
a) a compound of formula I 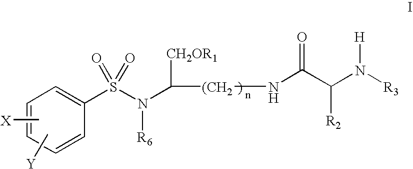
and pharmaceutically acceptable salts thereof,
wherein n is 3 or 4,
wherein X and Y, the same or different, are selected from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —OCF₃, —CN, —NO₂, —NR₄R₅, —NHCOR₄, —OR₄, —SR₄, —COOR₄, —COR₄, and —CH₂OH or X and Y together define an alkylenedioxy group selected from the group consisting of a methylenedioxy group of formula —OCH₂O— and an ethylenedioxy group of formula —OCH₂CH₂O—,
wherein R₆ is selected from the group consisting of a straight alkyl group of 1 to 6 carbon atoms, a branched alkyl group of 3 to 6 carbon atoms, a cycloalkylalkyl group having 3 to 6 carbon atoms in the cycloalkyl part thereof and 1 to 3 carbon atoms in the alkyl part thereof,
wherein R₃ is selected from the group consisting of H, a straight alkyl group of 1 to 6 carbon atoms, a branched alkyl group of 3 to 6 carbon atoms, a cycloalkyl group of 3 to 6 carbon atoms, and a group of formula R_{3A}—CO—, R_{3A} being selected from the group consisting of a straight or branched alkyl group of 1 to 6 carbon atoms, a cycloalkyl group having 3 to 6 carbon atoms, a cycloalkylalkyl group having 3 to 6 carbon atoms in the cycloalkyl part thereof and 1 to 3 carbon atoms in the alkyl part thereof, an alkyloxy group of 1 to 6 carbon atoms, tetrahydro-3-furanyloxy, —CH₂OH, —CF₃, —CH₂CF₃, —CH₂CH₂CF₃, pyrrolidinyl, piperidinyl, 4-morpholinyl, CH₃O₂C—, CH₃O₂CCH₂—, Acetyl-OCH₂CH₂—, HO₂CCH₂—, 3-hydroxyphenyl, 4-hydroxyphenyl, 4-CH₃OC₆H₄CH₂—, CH₃NH—, (CH₃)₂N—, (CH₃CH₂)₂N—, (CH₃CH₂CH₂)₂N—, HOCH₂CH₂NH—, CH₃OCH₂O—, CH₃OCH₂CH₂O—, C₆H₅CH₂O—, 2-pyrrolyl, 2-pyridyl, 3-pyridyl, 4-pyridyl-, 2-pyrazinyl, 2-quinolyl, 3-quinolyl, 4-quinolyl, 1-isoquinolyl, 3-isoquinolyl, 2-quinoxalinyl, a phenyl group of formula 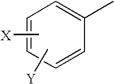
a picolyl group selected from the group consisting of 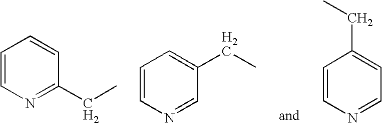
a picolyloxy group selected from the group consisting of 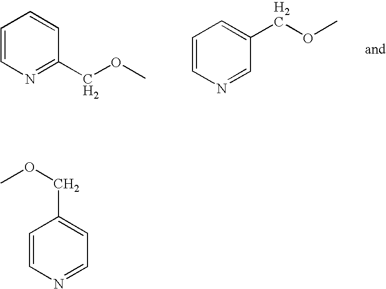
a substituted pyridyl group selected from the group consisting of 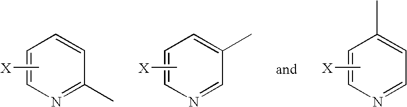
a group of formula 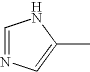
wherein X′ and Y′, the same or different, are selected from the group consisting of H, a straight alkyl group of 1 to 6 carbon atoms, a branched alkyl group of 3 to 6 carbon atoms, a cycloalkyl group of 3 to 6 carbon atoms, F, Cl, Br, l, —CF₃, —NO₂, —NR₄R₅, —NHCOR₄, —OR₄, —SR₄, —COOR₄, —COR₄ and —CH₂OH,
wherein R₄ and R₅, the same or different, are selected from the group consisting of H, a straight alkyl group of 1 to 6 carbon atoms, a branched alkyl group of 3 to 6 carbon atoms, and a cycloalkyl group of 3 to 6 carbon atoms,
wherein R₂ is selected from the group consisting of a diphenylmethyl group of formula IV 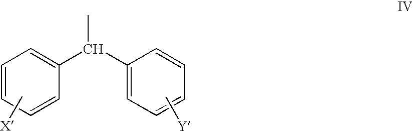
a naphthyl-1-CH₂— group of formula V 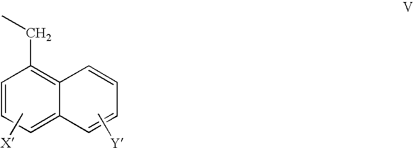
a naphthyl-2-CH₂— group of formula VI 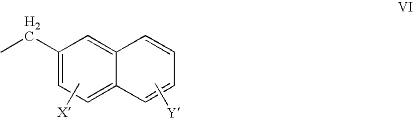
a biphenylmethyl group of formula VII 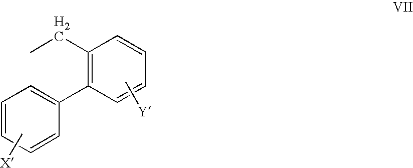
and an anthryl-9-CH₂— group of formula VIII 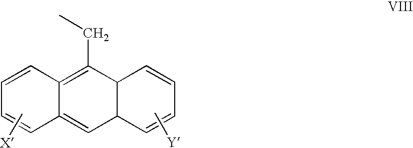
wherein R₁ is H or a physiologically cleavable unit and whereby upon physiological conditions said compound is converted into an active protease inhibitor;
b) a cytochrome P450 monooxigenase inhibitor, and;
c) a pharmaceutically acceptable carrier.

2. The pharmaceutical composition of claim 1, wherein R₁ is selected from the group consisting of H, (HO)₂P(O) and (MO)₂P(O), and a group of formula R_{1A}—CO—, R_{1A} being selected from the group consisting of a straight or branched alkyl group of 1 to 6 carbon atoms, a cycloalkyl group having 3 to 6 carbon atoms, a cycloalkylalkyl group having 3 to 6 carbon atoms in the cycloalkyl part thereof and 1 to 3 carbon atoms in the alkyl part thereof, an alkyloxy group of 1 to 6 carbon atom, —CH₂OH, CH₃O₂C—, CH₃O₂CCH₂—, Acetyl-OCH₂CH₂—, HO₂CCH2,2-hydroxyphenyl, 3-hydroxyphenyl, 4-hydroxyphenyl, (CH₃)₂NCH₂—, (CH₃)₂CHCH(NH₂)—, HOCH₂CH₂NH—, CH₃OCH₂O—, CH₃OCH₂CH₂O—, 2-pyrrolyl, 2-pyridyl, 3-pyridyl, 4-pyridyl, 1-methyl-1,4-dihydro-3-pyridyl, 2-pyrazinyl, 2-quinolyl, 3-quinolyl, 4-quinolyl, 1-isoquinolyl, 3-isoquinolyl, 2-quinoxalinyl, a phenyl group of formula 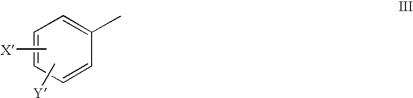
a picolyl group selected from the group consisting of 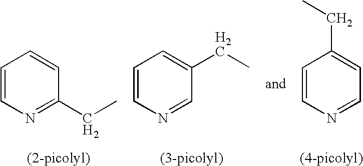
a picolyloxy group selected from the group consisting of 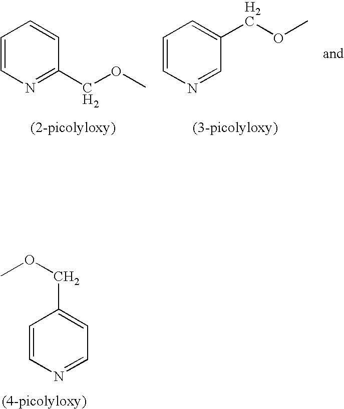
a substituted pyridyl group selected from the group consisting of 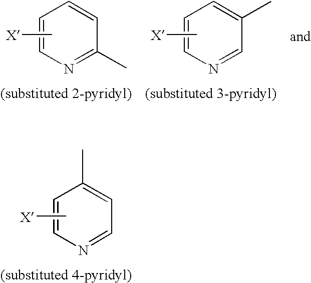
and a group of formula, 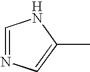
wherein M is an alkali metal or alkaline earth metal and wherein X′, Y′, R₄ and R₅ are as defined in claim 1.

3. A pharmaceutical composition comprising;
a) a compound of formula II, 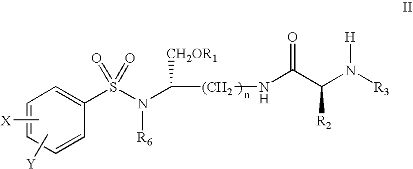
and pharmaceutically acceptable salts thereof,
wherein n is 3 or 4,
wherein X and Y, the same or different, are selected from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —OCF₃, —CN, —NO₂, —NR₄R₅, —NHCOR₄, —OR₄, —SR₄, —COOR₄, —COR₄, and —CH₂OH or X and Y together define an alkylenedioxy group selected from the group consisting of a methylenedioxy group of formula —OCH₂O— and an ethylenedioxy group of formula —OCH₂CH₂O—,
wherein R₆ is selected from the group consisting of a straight alkyl group of 1 to 6 carbon atoms, a branched alkyl group of 3 to 6 carbon atoms, a cycloalkylalkyl group having 3 to 6 carbon atoms in the cycloalkyl part thereof and 1 to 3 carbon atoms in the alkyl part thereof,
wherein R₃ is selected from the group consisting of H, a straight alkyl group of 1 to 6 carbon atoms, a branched alkyl group of 3 to 6 carbon atoms, a cycloalkyl group of 3 to 6 carbon atoms, and a group of formula R_{3A}—CO—, R_{3A} being selected from the group consisting of a straight or branched alkyl group of 1 to 6 carbon atoms, a cycloalkyl group having 3 to 6 carbon atoms, a cycloalkylalkyl group having 3 to 6 carbon atoms in the cycloalkyl part thereof and 1 to 3 carbon atoms in the alkyl part thereof, an alkyloxy group of 1 to 6 carbon atoms, tetrahydro-3-furanyloxy, —CH₂OH, —CF₃, —CH₂CF₃, —CH₂CH₂CF₃, pyrrolidinyl, piperidinyl, 4-morpholinyl, CH₃O₂C—, CH₃O₂CCH₂—, Acetyl-OCH₂CH₂—, HO₂CCH₂—, 3-hydroxyphenyl, 4-hydroxyphenyl, 4-CH₃OC₆H₄CH₂—, CH₃NH—, (CH₃)₂N—, (CH₃CH₂)₂N—, (CH₃CH₂CH₂)₂N—, HOCH₂CH₂NH—, CH₃OCH₂O—, CH₃OCH₂CH₂O—, C₆H₅CH₂O—, 2-pyrrolyl, 2-pyridyl, 3-pyridyl, 4-pyridyl-, 2-pyrazinyl, 2-quinolyl, 3-quinolyl, 4-quinolyl, 1-isoquinolyl, 3-isoquinolyl, 2-quinoxalinyl, a phenyl group of formula 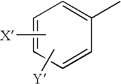
a picolyl group selected from the group consisting of 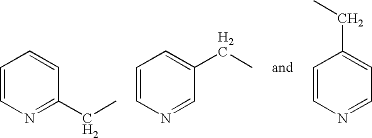
a picolyloxy group selected from the group consisting of 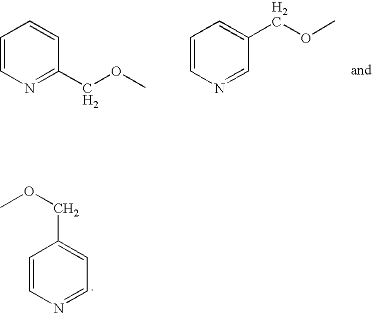
a substituted pyridyl group selected from the group consisting of 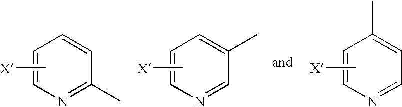
a group of formula 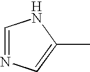
wherein X′ and Y′, the same or different, are selected from the group consisting of H, a straight alkyl group of 1 to 6 carbon atoms, a branched alkyl group of 3 to 6 carbon atoms, a cycloalkyl group of 3 to 6 carbon atoms, F, Cl, Br, I, —CF₃, —NO₂, —NR₄R₅, —NHCOR₄, —OR₄, —SR₄, —COOR₄, —COR₄ and —CH₂OH,
wherein R₄ and R₅, the same or different, are selected from the group consisting of H, a straight alkyl group of 1 to 6 carbon atoms, a branched alkyl group of 3 to 6 carbon atoms, and a cycloalkyl group of 3 to 6 carbon atoms,
wherein R₂ is selected from the group consisting of a diphenylmethyl group of formula IV 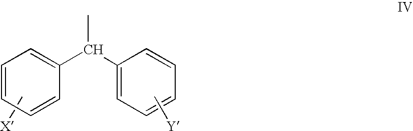
a naphthyl-1-CH₂— group of formula V 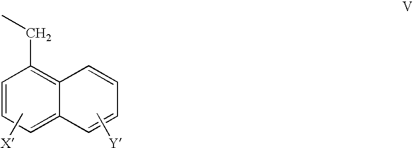
a naphthyl-2-CH₂— group of formula VI 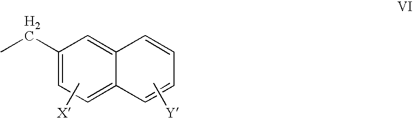
a biphenylmethyl group of formula VII 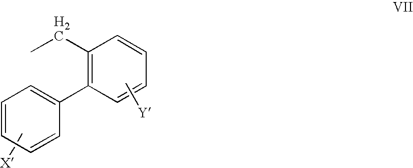
and an anthryl-9-CH₂— group of formula VIII 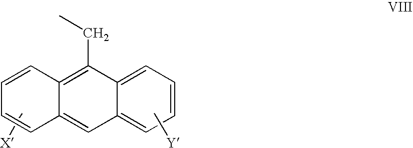
wherein R₁ is H or a physiologically cleavable unit, whereby upon physiological conditions said compound is converted into an active protease inhibitor;
b) a cytochrome P450 monooxigenase inhibitor, and;
c) a pharmaceutically acceptable carrier.

4. The pharmaceutical composition of claim 3, wherein R₁ is selected from the group consisting of H, (HO)₂P(O) and (MO)₂P(O), and a group of formula R_{1A}—CO—, R_{1A} being selected from the group consisting of a straight or branched alkyl group of 1 to 6 carbon atoms, a cycloalkyl group having 3 to 6 carbon atoms, a cycloalkylalkyl group having 3 to 6 carbon atoms in the cycloalkyl part thereof and 1 to 3 carbon atoms in the alkyl part thereof, an alkyloxy group of 1 to 6 carbon atoms, —CH₂OH, CH₃O₂C—, CH₃O₂CCH₂—, Acetyl-OCH₂CH₂—, HO₂CCH₂—, 2-hydroxyphenyl, 3-hydroxyphenyl, 4-hydroxyphenyl, (CH₃)₂NCH₂—, (CH₃)₂CHCH(NH₂)—, HOCH₂CH₂NH—, CH₃OCH₂O—, CH₃OCH₂CH₂O—, 2-pyrrolyl, 2-pyridyl, 3-pyridyl, 4-pyridyl, 1-methyl-1,4-dihydro-3-pyridyl, 2-pyrazinyl, 2-quinolyl, 3-quinolyl, 4-quinolyl, 1-isoquinolyl, 3-isoquinolyl, 2-quinoxalinyl, a phenyl group of formula 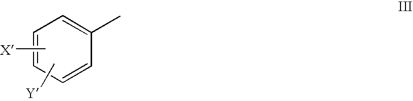
a picolyl group selected from the group consisting of 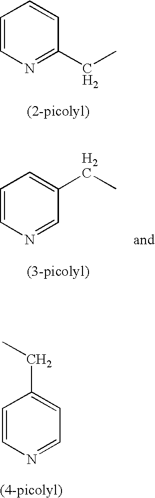
a picolyloxy group selected from the group consisting of 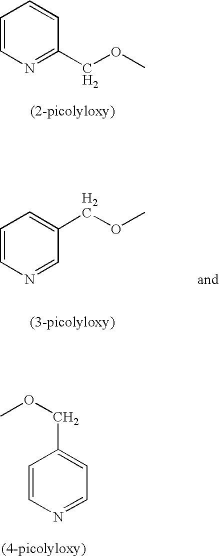
a substituted pyridyl group selected from the group consisting of 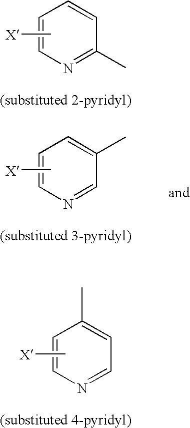
and a group of formula, 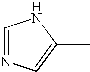
wherein M is an alkali metal or alkaline earth metal and wherein X′, Y′, R₄ and R₅ are as defined in claim 1.

5. The pharmaceutical composition of claim 4, wherein R₆ is iso-butyl and n is 3.

6. The pharmaceutical composition of claim 4, wherein R₆ is isobutyl and n is 4.

7. The pharmaceutical composition of claim 6, wherein R₁ is selected from the group consisting of H, (HO)₂P(O) and (NaO)₂P(O).

8. The pharmaceutical composition of claim 6, wherein R₁ is selected from the group consisting of CH₃CO, 3-pyridyl-CO, (CH₃)₂NCH₂CO and (CH₃)₂CHCH(NH₂)CO.

9. The pharmaceutical composition of claim 7, wherein R₃ is selected from the group consisting of CH₃CO, CH₃O—CO, (CH₃)₂N—CO, 3-pyridyl-CO, 4-pyridyl-CO and 4-morpholine-Co.

10. The pharmaceutical composition of claim 8, wherein R₃ is selected from the group consisting of CH₃CO, CH₃O—CO, (CH₃)₂N—CO, 3-pyridyl-CO, 4-pyridyl-CO and 4-morpholine-Co.

11. The pharmaceutical composition of claim 9, wherein X is 4-NH₂ and Y is H or F.

12. The pharmaceutical composition of claim 10, wherein X is 4-NH₂ and Y is H or F.

13. The pharmaceutical composition of claim 11, wherein R₂ is selected from the group consisting of a diphenylmethyl group of formula IV, a naphthyl-1-CH₂— group of formula V, a naphthyl-2-CH₂— group of formula VI, a biphenylmethyl group of formula VII and an anthryl-9-CH₂— group of formula VIII.

14. The pharmaceutical composition of claim 12, wherein R₂ is selected from the group consisting of a diphenylmethyl group of formula IV, a naphthyl-1-CH₂— group of formula V, a naphthyl-2-CH₂— group of formula VI, a biphenylmethyl group of formula VII and an anthryl-9-CH₂— group of formula VIII.

15. The pharmaceutical composition of claim 13, wherein R₂ is selected from the group consisting of a diphenylmethyl group of formula IV, a naphthyl-1-CH₂— group of formula V, and a naphthyl-2-CH₂— group of formula VI.

16. The pharmaceutical composition of claim 14, wherein R₂ is selected from the group consisting of a diphenylmethyl group of formula IV, a naphthyl-1-CH₂— group of formula V, and a naphthyl-2-CH₂— group of formula VI.

17. The pharmaceutical composition of claim 15, wherein X′ and Y′ is H.

18. The pharmaceutical composition of claim 16, wherein X′ and Y′ is H.

19. The pharmaceutical composition of claim 6 wherein R₂ is a diphenylmethyl group of formula IV.

20. The pharmaceutical composition of claim 19, wherein R₁ is selected from the group consisting of H, (HO)₂P(O) and (NaO)₂P(O).

21. The pharmaceutical composition of claim 19, wherein R₁ is selected from the group consisting of CH₃CO, 3-pyridyl-CO, (CH₃)₂NCH₂CO and (CH₃)₂CHCH(NH₂)CO.

22. The pharmaceutical composition of claim 20, wherein R₃ is selected from the group consisting of CH₃CO, CH₃O—CO, (CH₃)₂N—CO, 3-pyridyl-CO, 4-pyridyl-CO and 4-morpholine-CO.

23. The pharmaceutical composition of claim 21, wherein R₃ is selected from the group consisting of CH₃CO, CH₃O—CO, (CH₃)₂N—CO, 3-pyridyl-CO, 4-pyridyl-CO and 4-morpholine-CO.

24. The pharmaceutical composition of claim 22, wherein X is 4-NH₂ and Y is H or F.

25. The pharmaceutical composition of claim 23, wherein X is 4-NH₂ and Y is H or F.

26. The pharmaceutical composition of claim 22, wherein X is 4-NH₂, Y is H, X′ is H, Y′ is H and R₃ is CH₃O—CO.

27. The pharmaceutical composition of claim 26, wherein R₁ is (HO)₂P(O).

28. The pharmaceutical composition of claim 26, wherein R₁ is (NaO)₂P(O).

29. The pharmaceutical composition of claim 26, wherein R₁ is H.

30. The pharmaceutical composition of claim 22, wherein X is 4-NH₂, Y is 3-F, X′ is H, Y′ is H and R₃ is CH₃O—CO.

31. The pharmaceutical composition of claim 30, wherein R₁ is (HO)₂P(O).

32. The pharmaceutical composition of claim 30, wherein R₁ is (NaO)₂P(O).

33. The pharmaceutical composition of claim 30, wherein R₁ is H.

34. The pharmaceutical composition of claim 22, wherein X is 4-NH₂, Y is H or 3-F, X′ is H, Y′ is H and R₃ is CH₃CO.

35. The pharmaceutical composition of claim 34, wherein R₁ is (HO)₂P(O).

36. The pharmaceutical composition of claim 34, wherein R₁ is (NaO)₂P(O).

37. The pharmaceutical composition of claim 34, wherein R₁ is H.

38. The pharmaceutical composition of claim 22, wherein X is 4-NH₂, Y is H or 3-F, X′ is H, Y′ is H and R₃ is 4-morpholine-CO.

39. The pharmaceutical composition of claim 23, wherein X is 4-NH₂, Y is H, X′ is H, Y′ is H and R₃ is CH₃O—CO.

40. The pharmaceutical composition of claim 39, wherein R₁ is 3-pyridyl-CO.

41. The pharmaceutical composition of claim 39, wherein R₁ is (CH₃)₂NCH₂CO.

42. The pharmaceutical composition of claim 39, wherein R₁ is (CH₃)₂CHCH(NH₂)CO.

43. The pharmaceutical composition of claim 39, wherein R₁ is CH₃CO.

44. The pharmaceutical composition of claim 17, wherein R₂ is Naphtyl-1-CH₂—, Y is H and R₃ is 4-morpholine-CO.

45. The pharmaceutical composition of claim 17, wherein R₂ is Naphtyl-2-CH₂—, Y is H and R₃ is CH₃O—CO.

46. The pharmaceutical composition of claim 1, wherein said cytochrome P450 monooxigenase inhibitor is selected from the group consisting of ritonavir (RTV), ketoconazole, fluconazole, nefazodone, fluvoxamine, fluoxetine, macrolide antibiotics, sertraline sulfaphenazole and erythromycin.

47. The pharmaceutical composition of claim 1, wherein said composition is administered orally.

48. The pharmaceutical composition of claim 1, wherein said composition is administered twice-daily.

49. A kit for treating or preventing an HIV infection or for treating or preventing AIDS, the kit comprising a) a first container containing a compound of formula I; 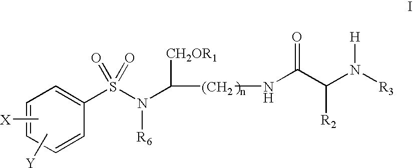
or a pharmaceutically acceptable salts thereof
wherein n, X, Y, X′, Y′, R₁, R₂, R₃, R₄, R₅ and R₆ are as defined in claim 1 and a second container containing a cytochrome P450 monooxigenase inhibitor or;
b) a container comprising both the compound of formula I and the CYP450 inhibitor.

50. The kit as defined in claim 49, wherein R₁ is as defined in claim 2.

51. The kit as defined in any claim 49, wherein said cytochrome P450 monooxigenase inhibitor is selected from the group consisting of ritonavir (RTV), ketoconazole, fluconazole, nefazodone, fluvoxamine, fluoxetine, macrolide antibiotics, sertraline sulfaphenazole and erythromycin.

52. A method of treating or preventing an HIV infection or of treating or preventing AIDS, the method comprising administering a pharmaceutical composition as defined in claim 1 to a mammal in need thereof.

53. The method of claim 52, wherein said composition is administered orally.

54. The method of claim 52, wherein said composition is administered twice-daily.

55. The use of a pharmaceutical composition as defined in claim 1 for the treatment or prevention of an HIV infection or for the treatment or prevention of AIDS.

56. A method of treating or preventing an HIV infection or of treating or preventing AIDS, the method comprising administering
a) a compound of formula I 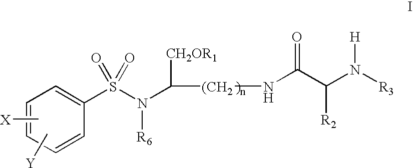
wherein n, X, Y, X′, Y′, R₁, R₂, R₃, R₄, R₅ and R₆ are as defined in claim 1 or a pharmaceutically acceptable salts thereof, and;
b) one or more CYP450 inhibitor in an amount which is sufficient to reduce the metabolism of the compound of formula I.

57. The method as defined in claim 56, wherein said CYP450 inhibitor is selected from the group consisting of ritonavir (RTV), ketoconazole, fluconazole, nefazodone, fluvoxamine, fluoxetine, macrolide antibiotics, sertraline sulfaphenazole and erythromycin.

58. The method of claim 56, wherein administration of the compound of formula I and the CYP450 inhibitor is performed separately, simultaneously or sequentially.

59. The method of claim 58, wherein the administration of the compound of formula I and the CYP450 inhibitor is performed by administering a) a first pharmaceutical composition comprising a compound of formula I and a pharmaceutically acceptable carrier and b) a second pharmaceutical composition comprising a CYP450 inhibitor and a pharmaceutically acceptable carrier.

60. The method of claim 58, wherein the administration of the compound of formula I and the CYP450 inhibitor is performed by administering a single pharmaceutical composition comprising a compound of formula I, a CYP450 inhibitor and a pharmaceutically acceptable carrier.

61. The method of claim 60, wherein said CYP450 inhibitor is ritonavir.

62. A method for improving the pharmacokinetics of a compound of formula I as defined in claim 1, the method comprising administering to a human in need thereof, the compound of formula I and an amount of a CYP450 inhibitor effective to inhibit cytochrome P450 monooxygenase.

63. The pharmaceutical composition of claim 1, wherein the ratio of the compound of formula I over the cytochrome P450 monooxygenase inhibitor is between about 1:1 to about 10:1.

64. The pharmaceutical composition of claim 63, wherein the ratio is between about 3:1 and about 6:1.

65. A method of inhibiting an HIV having a reduced susceptibility to a protease inhibitor other than the protease inhibitor defined by formula I, the method comprising administering a compound of formula I alone or in combination with a CYP450 inhibitor to an individual in need thereof.

66. The method of claim 65, wherein said HIV is HIV-1.

67. The method of claim 66, wherein said HIV has a reduced susceptibility to one or more of a protease inhibitor selected from the group consisting of Atazanavir, Amprenavir, Indinavir, Lopinavir, Nelfinavir, Ritonavir and Saquinavir.

68. The method of claim 67, wherein said HIV-1 possesses an aspartyl protease having one or more mutations.
